(19) United States
(12) Patent Application Publication
(10) Pub. No.: US 2026/0028482 A1
(43) Pub. Date: Jan. 29, 2026

(54) Substitution at the Central Position of Heptamethine Dyes By Aryllithium Addition for Improved Fluorophores

(71) Applicant: Board of Trustees of the University of Arkansas, Little Rock, AR (US)

(72) Inventors: Shang Jia, Fayetteville, AR (US); Lei Guo, Fayetteville, AR (US)

(21) Appl. No.: 19/279,610

(22) Filed: Jul. 24, 2025

Related U.S. Application Data
(60) Provisional application No. 63/674,989, filed on Jul. 24, 2024.

(51) Int. Cl. C09B 23/08; C07D 209/08; C07D 209/12; C07D 209/24; C07D 209/60; C07D 311/58; C07D 335/02; C09K 11/06

(52) U.S. Cl. CPC C09B 23/083; C07D 209/08; C07D 209/12; C07D 209/24; C07D 209/60; C07D 311/58; C07D 335/02; C09K 11/06; C09K 2211/1007; C09K 2211/1018

(57) ABSTRACT

A method of synthesizing a fluorophore is disclosed. The method includes the steps of converting a heptamethine dye to a keto-heptamethine dye and converting the keto-heptamethine dye to a 4′-aryl heptamethine dye.

### CROSS-REFERENCE TO RELATED APPLICATIONS

This application claims the benefit of U.S. Provisional Application No. 63/674,989, entitled “Substitution at the Central Position of Heptamethine Dyes by Aryllithium Addition for Improved Fluorophores” and filed on Jul. 24, 2024. The complete disclosure of said provisional application is hereby incorporated by reference.

### STATEMENT REGARDING FEDERALLY SPONSORED RESEARCH OR DEVELOPMENT

Not applicable

### BACKGROUND OF THE INVENTION

Polymethine dyes, characterized by two heterocycles connected by odd numbers of methine units, are a major family of dyes with more than 150 years of history. In particular, heptamethine fluorophores, which are the polymethine dyes with seven methine units (FIG. 1), have gained significant interest due to their favorable near-infrared (NIR, 700-1000 nm) excitation and emission properties that enable deeper penetration and reduced background than visible fluorophores, making them highly suitable for in vivo imaging applications. The success of indocyanine green (ICG) in various clinical settings exemplifies the potential of this fluorophore family. Furthermore, the recent FDA approval of OTL-38, a folate receptor-targeted heptamethine conjugate, underscores the growing impact of these dyes in clinical translation. Moreover, by strategically incorporating specific heterocycles in the scaffold, researchers can achieve even longer emission wavelengths, extending into the shortwave-infrared (SWIR, 1000-2000 nm, also known as NIR-II) region for in vivo imaging with deeper penetration and extraordinary contrast.

As with any fluorophore, heptamethine dyes require modification prior to biological applications to introduce proper physical property, chemical behavior, and/or biological activity. In particular, the planar conjugated systems in heptamethine dyes result in strong tendency towards non-emissive aggregation with little solubility in water, which requires the modification with hydrophilic groups and/or steric bulk to allow their imaging in aqueous conditions. Additionally, the stability of heptamethine dyes can introduce complexity in their imaging applications. For example, heptamethine dyes are photo-bleached relatively fast under illumination compared to other family of fluorophores, and many, including ICG, have been reported to degrade in the presence of physiological nucleophiles or even in simple aqueous solutions. While these limitations can be partially overcome by using formulation methods, the heterogeneous nature of formulation brings about batch-to-batch variations as well as potential in vivo instability. Therefore, structural modification of heptamethine fluorophores is often essential to enhance their physical and chemical properties for reliable biological applications in physiological conditions.

Despite the necessity of heptamethine dye modifications, the limited scope of modification methods remains as a hurdle to even broader utilization of these fluoro-phores. In this context, late-stage functionalization of existing heptamethine dyes primarily focuses on the replacement of center chloride in the popular 4′-chloro heptamethine scaffold, which is synthesized from the condensation between two heterocyclic salt moiety and an accessible Schiff base bearing the chloride (FIG. 1). Such replacement can take place via the substitution reaction with N, O or S nucleophiles, whose products can be labile under physiological conditions, or coupling reactions to form robust C—C bonds, although the conversion usually requires high temperature and prolonged reaction time that can be harmful to the fluorophore (FIG. 1). On the other hand, more complex modification on the polymethine chain, including bulky 4′-substitutions or substitutions on other positions, requires the bottom-up synthesis of the fluorophore core, such as the use customized Schiff base linkage carrying such modifications. Recent development using the ring-opening of pyridium reagents in place of Schiff base offers a viable way to build fluorophores with one or more substituents on the polymethine chain (FIG. 9B-C). However, there are uncertainties in synthesizing fluorophore cores with these customized building blocks, as many heptamethine dyes with demanding heterocycles requires different reactions (i.e. selection of base, solvent, temperature and reaction time) and rigorous purification even with established Schiff base building blocks. As such, a mild and effective modification method for heptamethine dyes is highly desired to facilitate the tuning of these fluorophores towards various imaging requirements.

We herein report an efficient aryllithium addition pathway to introduce various aryl substitution to the 4′-position of polymethine dyes inspired by the synthesis of xanthene dyes. Xanthene dyes, such as rhodamine and fluorescein, can be constructed from the nucleophilic addition between a xanthone and an aryl carboanion, usually obtained from an aryl bromide after a lithium-halogen exchange, followed by acidic dehydration (FIG. 1). This popular transformation has furnished a variety of xanthene dye derivatives, including red-shifted fluorophores with heteroatom exchange and has been incorporated in many fluorescent probes for advanced bioimaging and biosensing. Inspired by these successes, we have noticed the structural similarity between the xanthone substrate in xanthene dye synthesis and the keto-form of heptamethine dyes, the S_{RN}1 hydrolysis product of common 4′-chloro heptamethine fluorophores, and discovered that such lithium addition reaction can be used to construct 4′-aryl modified heptamethine dyes (FIG. 1). Using this strategy, we show our successful preparation of a panel of 4′-aryl substituted heptamethine fluorophores starting from the 4′-chloro precursor. This conversion is compatible with a variety of functional groups and tolerates moieties with strong steric demands, affording ortho-disubstituted aryl-substitution at the 4′-position of the heptamethine fluorophore (heptamethine-X), which benefits from enhanced stability and reduced aggregation but is otherwise inaccessible from palladium-catalyzed modifications. We finally showcase our strategy by a facile synthesis of an IR-780 derivative that carries two octadecyl chains as a fluorogenic membrane marker with higher contrast over commercial DiR stain. Collectively, this xanthene-dye inspired synthesis of 4′-modified polymethine fluorophores serves as a further step in unifying the design and synthetic principles between these two families towards the make of next-generation fluorophores with combined benefits.

### BRIEF SUMMARY OF THE INVENTION

As a major family of red-shifted fluorophores that operate beyond the visible light, polymethine dyes are pivotal in light-based biological techniques. However, the methods for tuning this kind of fluorophores by structural modification remain restricted to bottom-up synthesis and modification using coupling or nucleophilic substitutions. In this study, we introduce a two-step, late-stage functionalization process for heptamethine dyes. This process enables the substitution of the central chloride atom in the commonly used 4′-chloro heptamethine scaffold with various aryl groups using aryllithium reagents. This method borrows the building block and designs from the xanthene dye community, and offers a mild and convenient way for the diversification of heptamethine fluorophores. Notably, this efficient conversion allows for the synthesis of heptamethine-X, the heptamethine scaffold with two ortho-substituents on the 4′-aryl modification, which brings enhanced stability and reduced aggregation to the fluorophore. We showcase the utility of this synthesis by a facile synthesis of a fluorogenic, membrane-localizing fluorophore that outperforms the commercial counterparts with higher brightness and contrast. Overall, this method establishes the synthetic similarities between polymethine and xanthene fluorophores, and provides reference for future optimizing heptamethine fluorophores for their biological applications.

Proof of Concept: We show that this conversion is compatible with a variety of functional groups and tolerates moieties with strong steric demands, affording 2,6-disubstituted aryl-substitution at the 4′-position of the fluorophore, which is impractical with palladium-catalyzed modifications. The resulting dyes with such steric bulk around polymethine chains show improved stability and reduced aggregation tendency. Moreover, we successfully applied this synthetic method to heptamethine fluorophores with red-shifted heterocycles with excitation/emission beyond 1000 nm, further confirming the broad compatibility of this pathway. We finally showcase our strategy by creating an IR-780 derivative that carries two octadecyl chains as a fluorogenic membrane marker with higher contrast and enhanced photostability over commercially DiR stain. Collectively, this xanthene-dye inspired synthesis of 4′-modified polymethine serves as a further step in unifying the design and synthetic principles between the polymethine and xanthene dye families towards the make of next-generation fluorophores with combined benefits.

In one respect, the present invention is directed to a method of synthesizing a fluorophore, comprising the steps of: (a) converting a heptamethine dye to a keto-heptamethine dye; and (2) converting the keto-heptamethine dye to a 4′-aryl heptamethine dye. The heptamethine dye of the present method has the formula:

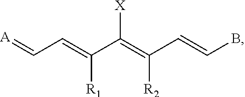

wherein: X is selected from chloride (Cl), bromine (Br), and iodine (I); R₁ and R₂ are each independently selected from hydrogen (H), an alkyl, and a halogen, or R₁ and R₂ together complete a cycloalkenyl ring, a heterocyclyl ring, or a polycyclyl ring system; and A and B are each independently selected from a bicyclic heterocycle, a tricyclic heterocycle, and a tetracyclic heterocycle. The heptamethine may be IR-780, IR-783, IR-775, Chrom7, or IR-1061.

The keto-heptamethine dye of the present method has the formula:

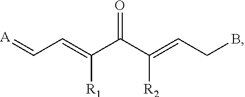

wherein: X is selected from chloride (Cl), bromine (Br), and iodine (I); R₁ and R₂ are each independently selected from hydrogen (H), an alkyl, and a halogen, or R₁ and R₂ together complete a cycloalkenyl ring, a heterocyclyl ring, or a polycyclyl ring system; and A and B are each independently selected from a bicyclic heterocycle, a tricyclic heterocycle, and a tetracyclic heterocycle. The keto-heptamethine dye may be IR-780═O, IR-783═O, IR-775═O, Chrom7═O, or IR-1061=O.

The 4′-aryl heptamethine dye of the present method has the formula:

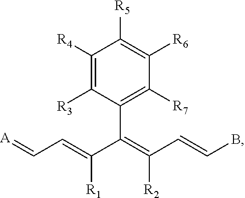

wherein: R₁ and R₂ are each independently selected from hydrogen (H), an alkyl, and a halogen, or R₁ and R₂ together complete a cycloalkenyl ring, a heterocyclyl ring, or a polycyclyl ring system; R₃-R₇ are independently selected from hydrogen, an alkyl, an alkoxy (such as methoxy), a haloalkyl (such as trifluoromethyl), an amine (such as dialkylamine), and a halogen; and A and B are each independently selected from a bicyclic heterocycle, a tricyclic heterocycle, and a tetracyclic heterocycle. The 4′-aryl heptamethine dye may be IR780-Ph, IR780-Ph(Me), IR780-Ph(OMe), IR780-Ph(CH₂OH), IR780-Ph(COOH), IR780-Ph(CH₂StBu), IR780-Ph(2Me), IR780-Ph(2iPr), IR780-Ph(2CH₂OH), IR775-Ph(Me), IR813-Ph(Me), Chrom7-Ph(Me), IR1061-Ph(Me), or IR783-Ph(Me).

The step of converting the keto-heptamethine dye to the 4′-aryl heptamethine dye includes the step of reacting the keto-heptamethine dye with an aryl anion. The aryl anion is generated via reduction of an aryl halide with a metal or via a metal-halo exchange reaction. The aryl anion may be an aryl lithium reagent, an aryl Grignard reagent, or combinations thereof. In one embodiment, the aryl lithium reagent may be prepared by reacting n-BuLi with an aryl halide selected from the group of bromobenzene, 2-bromotoluene, 1-bromo-2-isopropylbenzene, 2-bromoanisole, ((2-bromo-3-methylbenzyl)oxy)trimethylsilane, tert-butyl 2-bromobenzoate, or (2-bromobenzyl)(tert-butyl)sulfane. In an alternative embodiment, the aryl lithium reagent may be prepared by reacting t-BuLi with 2-bromo-1,3-dimethylbenzene, 1,3-Bis((allyloxy)methyl-2-bromobenzene, or 2-bromo-1,3-diisopropylbenzene.

In one embodiment, the step of converting the heptamethine dye to keto-heptamethine dye includes the step of reacting said 4′-chloro heptamethine dye with sodium acetate and dimethylformamide. In an alternative embodiment, the step of converting the heptamethine dye to keto-haptamethine dye includes the step of reacting said 4′-chloro heptamethine dye with NHS and DIPEA.

These and other features, objects and advantages of the present invention will become better understood from a consideration of the following detailed description of the preferred embodiments and appended claims in conjunction with the drawings as described following:

### BRIEF DESCRIPTION OF THE DRAWINGS

FIG. 1 illustrates heptamethine fluorophore synthesis and its 4′-aryl modification with conventional method (top) and the present strategy (middle), which is inspired by the synthesis of xanthene dyes (bottom).

FIGS. 2A-2E illustrate 4′-Aryl modification on heptamethine for reduced aggregation: (FIG. 2A) Schematic showing the reduction of aggregation by introducing bulky and pointy attachment to the planar dye; Normalized absorption spectra of increasing concentrations of (FIG. 2B) IR-780, (FIG. 2C) IR780-Ph, (FIG. 2D) IR780-Ph(CH₂OH), and (FIG. 2E) IR780-Ph(2CH₂OH) in PBS. Normalized spectra are shown in FIGS. 14A-14D.

FIGS. 3A-3B illustrate improved stability of new IR-780 derivatives with 4′-aryl substitutions: (FIG. 3A) Degradation of dyes over time in bovine calf serum at 37° C. in the dark. IR-780-BSA refers to the dye-serum protein conjugate; (FIG. 3B) Half-lives of dyes in of dyes in 1:1 methanol/water solution under 730 nm LED illumination (6.8 mW/cm²); see FIGS. 15A-15B for photobleaching curves, dye absorption and LED luminescence profile.

FIGS. 4A-4E are imaging of cell membrane with IR780-2C18: (FIG. 4A) Structure of IR780-2C18; (FIG. 4B) Relative fluorescence intensity of IR780-2C18 (1 μM) in water and alcohol solvents, normalized to the intensity in water solution; (FIG. 4C) Absorption and emission spectra of IR780-2C18 (1 μM) in water with or without preformed liposomes (200 μM, 55:45 DPPC/CHOL); (FIG. 4D) Epifluorescence imaging of cell membrane on A549 cells using 1.7 μM DiI and IR780-2C18. Scale-bar: 20 μm; (FIG. 4E) Comparison of mean cellular signal to background ratio for cells incubated with 0.5 μM DiR or IR780-2C18 under same imaging condition. Data are calculated on 10 cells from four independent incubations for each dye. *** P≤0.001; two-tailed Student's t-test.

FIG. 5 is an overview schematic of the present invention.

FIG. 6 illustrates the synthesis of 4′-aryl substituted IR-780 derivatives: ^{a} Isolated yields; ^{b} Reaction condition: aryl bromide (12 equiv.) was reacted with n-BuLi (8 equiv.) in THF at −84° C. for 10 min, then IR780═O (1 equiv.) was added and reacted at room temperature for 30 min before quenching with HCl; ^{c} R²′=OTMS; ^{d} R²′=COOt-Bu; ^{e} Reaction condition: same as b, except aryl bromide (0.34 mmol) was reacted with t-BuLi (0.68 mmol) for 40 min; ^{f} Synthesized from deprotection of 2 h, overall yield listed.

FIG. 7 illustrates synthesis of 4′-(2-methyl)phenyl substituted heptamethine dyes with different heterocycles (Scheme 2). Reaction conditions for step 1: 4′-chloroheptametine dye (1 equiv.), N-hydroxysuccinimide (NHS, 3 equiv.) and N,N-diisopropylethylamine (DIPEA, 3.0 equiv.) were reacted under N₂ in DMF until starting dye was consumed by TLC. For step 2:2-Bromotoluene (12 equiv.) were reacted with n-BuLi (8 equiv.) or t-BuLi (24 equiv.) in THF at −84° C. for 10 min, then keto-dyes (1 equiv.) was added and reacted at room temperature for 30 min before quenching with HCl. Isolated yields are listed for both steps.

FIG. 8 illustrates photophysical properties of Cy7 derivatives: ^{a} See FIG. 18 for error values. ^{b} ICG in ethanol (ϕF=0.132)^{59,60} was used as a reference.

FIGS. 9A-9C illustrate existing synthetic methods for indolinium heptamethine cyanine (Cy7) fluorophores with modification on the methine linkage. Synthesis of modified Cy7 dyes from custom Schiff bases (FIG. 9A), from Zincke salts (FIG. 9B), and from pyridinium benzoxazoles (FIG. 9C).

FIG. 10 illustrates structures of commercially-available or previously-reported heptamethine dyes referenced in this invention.

FIGS. 11A-11L illustrate normalized absorption and emission spectra of ethanol solution containing 2 μM (FIG. 11A) IR-780, (FIG. 11B) IR780-Ph, (FIG. 11C) IR780-Ph(Me), (FIG. 11D) IR780-Ph(iPr), (FIG. 11E) IR780-Ph(CH2OH), (FIG. 11F) IR780-Ph(OMe), (FIG. 11G) IR780-Ph(COOH), (FIG. 11H) IR780-Ph(CH2StBu), (FIG. 11I) IR780-Ph(2Me), (FIG. 11J) IR780-Ph(2iPr), (FIG. 11K) IR780-Ph(CH2OAllyl) and (FIG. 11L) IR780-Ph(2CH2OH).

FIGS. 12A-12B illustrate combined (FIG. 12A) absorption and (FIG. 12B) emission spectra of IR-780 and its derivatives in ethanol.

FIGS. 13A-13G illustrate normalized absorption and emission spectra of water solution containing 2 μM (FIG. 13A) IR-780, (FIG. 13B) IR780-Ph, (FIG. 13C) IR780-Ph(CH2OH), (FIG. 13D) IR780-Ph(2CH2OH) and (FIG. 13E) IR780-Ph(COOH). Combined absorption and emission spectra are shown in (FIG. 13F) and (FIG. 13G), respectively.

FIGS. 14A-14D illustrate the reduction of aggregation by 4′-aryl modifications. (a-d) Normalized absorption spectra of increasing concentrations of (FIG. 14A) IR-780, (FIG. 14B) IR780-Ph, (FIG. 14C) IR780-Ph(CH₂OH), and (FIG. 14D) IR780-Ph(2CH₂OH) in PBS. Absorbance smaller than 2 was measured through a 1 cm light path. Absorbance larger than 2 was measured through a 0.5 cm light path and doubled to represent absorbance at 1 cm.

FIGS. 15A-15B illustrate the photobleach experiment of IR-780 and its derivatives. (FIG. 15A) Photobleach curves of dyes in 1:1 methanol/water solution under 730 nm LED illumination (6.8 mW/cm2) determined by reduction of their absorption over time. Results are shown as mean±s.d. (n=3). (FIG. 15B) Comparison of absorption spectra of dyes in 1:1 methanol/water for photobleach experiment and the spectrum of LED light source.

FIGS. 16A-16H illustrate absorption and emission of IR780-2C18 in different conditions. Absorption (FIG. 16A) and emission (FIG. 16B) spectra of 2 μM IR780-2C18 in alcoholic solvents. Absorption (FIG. 16C) and emission (FIG. 16D) spectra of IR780-2C18 in solvents with varying polarity. Absorption (FIG. 16E) and emission (FIG. 16F) spectra of IR780-2C18 in ethanol with varying concentrations of HCl. Absorption (FIG. 16G) and emission (FIG. 16H) spectra of IR780-Ph with varying concentrations of HCl for comparison. HCl stock solution (200 mM) was prepared by adding cold ethanol into acetyl chloride.

FIGS. 17A-17C illustrate the comparison of membrane imaging using DiR and IR780-2C18. Representative images of cell stained with 2.5 μM DiR (FIG. 17A) and IR780-2C18 (FIG. 17B). The brightness and contrast in (FIG. 17A) and (FIG. 17B) are adjusted to show the staining of the cell. Scale-bar: 20 μm. (FIG. 17C) Mean fluorescence intensity in A549 cells after incubation with 2.5 μM DiR or IR780-2C18 under same imaging condition after background subtraction. Data are calculated on 10 cells from four independent incubations for each dye. *** P≤0.001; two-tailed Student's t-test.

FIG. 18 is a table illustrating the photophysical properties of Cy7 derivatives. ^{a} ICG in ethanol (ϕ_{F}=0.132) was used as a reference.

FIG. 19 is a table illustrating the Absorption and emission maxima of 3a-d and their starting 4′-chloro dyes in dichloromethane. ^{a} Not determined due to fluorometer wavelength limit. ^{b} Measured in methanol.

FIG. 20 is an 1H NMR spectrum of S1.

FIG. 21 are 1H and 13C NMR spectra of S2.

FIG. 22 are 1H and 13C NMR spectra of S3.

FIG. 23 are 1H and 13C NMR spectra of IR780═O.

FIG. 24 are 1H and 13C NMR spectra of IR775═O.

FIG. 25 are 1H and 13C NMR spectra of IR813═O.

FIG. 26 are 1H and 13C NMR spectra of Chrom7═O.

FIG. 27 are 1H and 13C NMR spectra of IR1061═O.

FIG. 28 are 1H and 13C NMR spectra of IR780-Ph.

FIG. 29 are 1H and 13C NMR spectra of IR780-Ph(Me).

FIG. 30 are 1H and 13C NMR spectra of IR780-Ph(CH2OH).

FIG. 31 are 1H and 13C NMR spectra of IR780-Ph(OMe).

FIG. 32 are 1H and 13C NMR spectra of IR780-Ph(COOH).

FIG. 33 are 1H and 13C NMR spectra of IR780-Ph(CH2StBu).

FIG. 34 are 1H and 13C NMR spectra of IR775-Ph(Me).

FIG. 35 are 1H and 13C NMR spectra of IR813-Ph(Me).

FIG. 36 are 1H and 13C NMR spectra of Chrom7-Ph(Me).

FIG. 37 are 1H and 13C NMR spectra of IR1061-Ph(Me).

FIG. 38 are 1H and 13C NMR spectra of IR780-Ph(2Me).

FIG. 39 are 1H and 13C NMR spectra of IR780-Ph(2CH2OAllyl).

FIG. 40 are 1H and 13C NMR spectra of IR780-Ph(2CH2OH).

FIG. 41 are 1H and 13C NMR spectra of IR780-Ph(2C18).

FIG. 42 are 1H and 13C NMR spectra of IR783═O.

FIG. 43 are 1H and 13C NMR spectra of IR780-Ph(iPr).

FIG. 44 are 1H and 13C NMR spectra of IR780-Ph(2iPr).

FIG. 45 are 1H and 13C NMR spectra of IR783-Ph(Me).

### DETAILED DESCRIPTION OF THE INVENTION

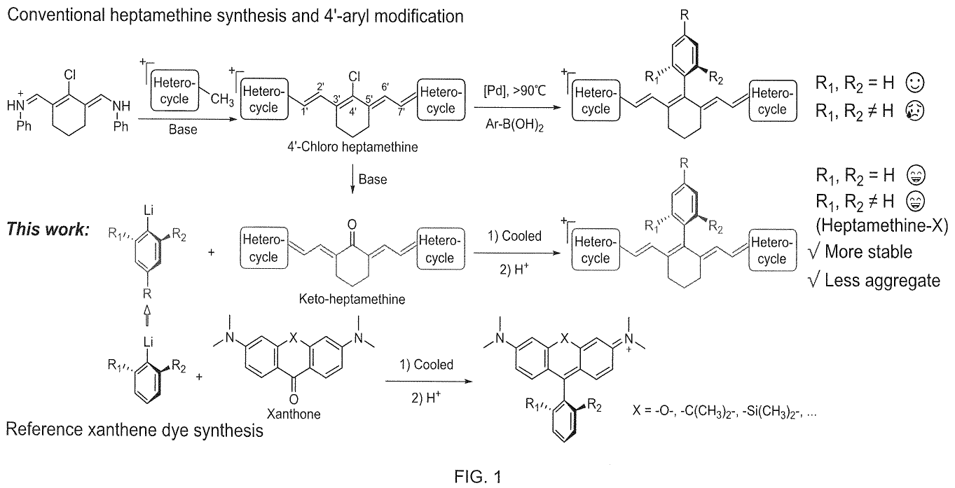

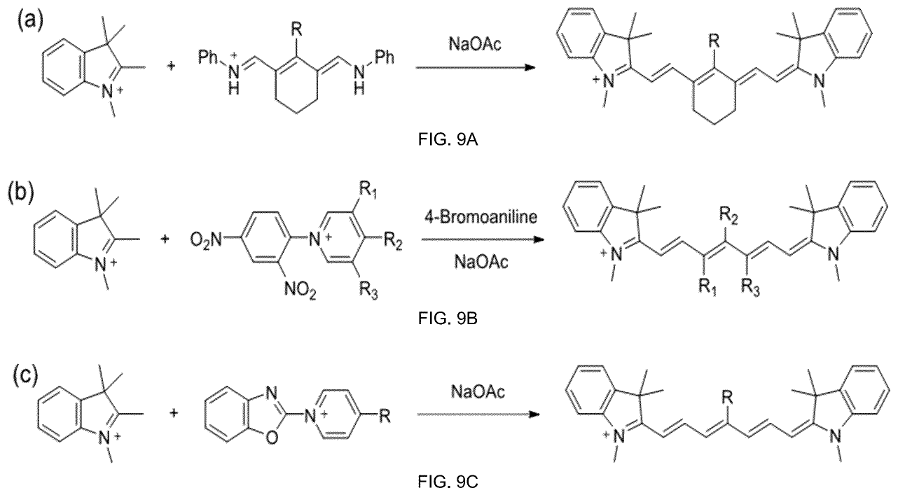

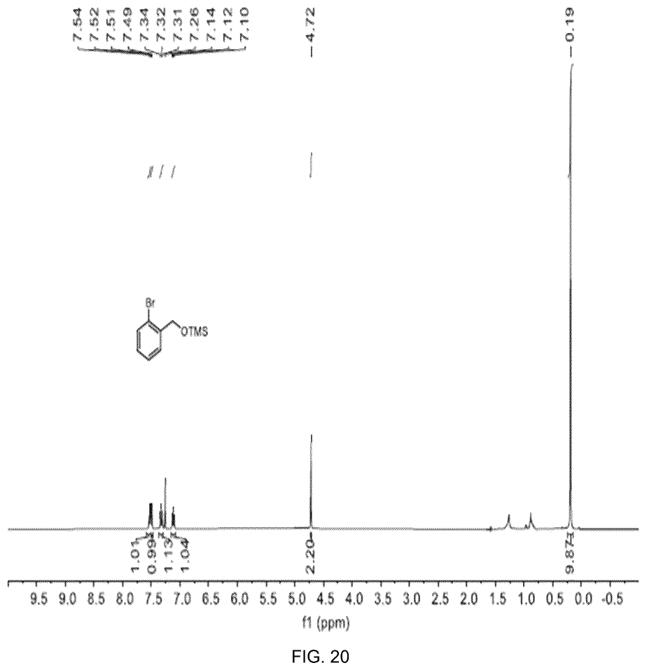

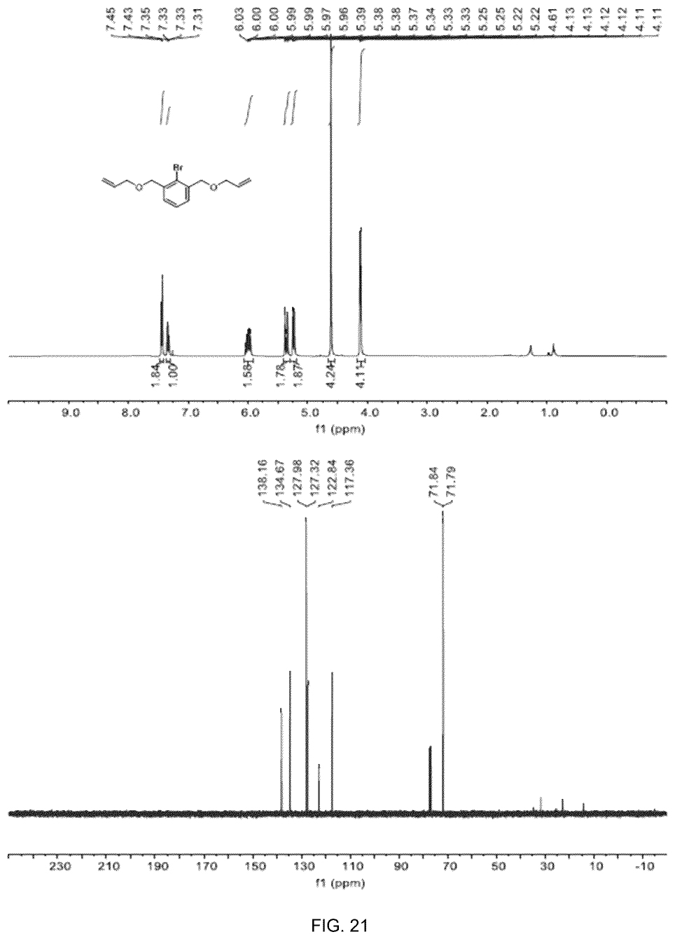

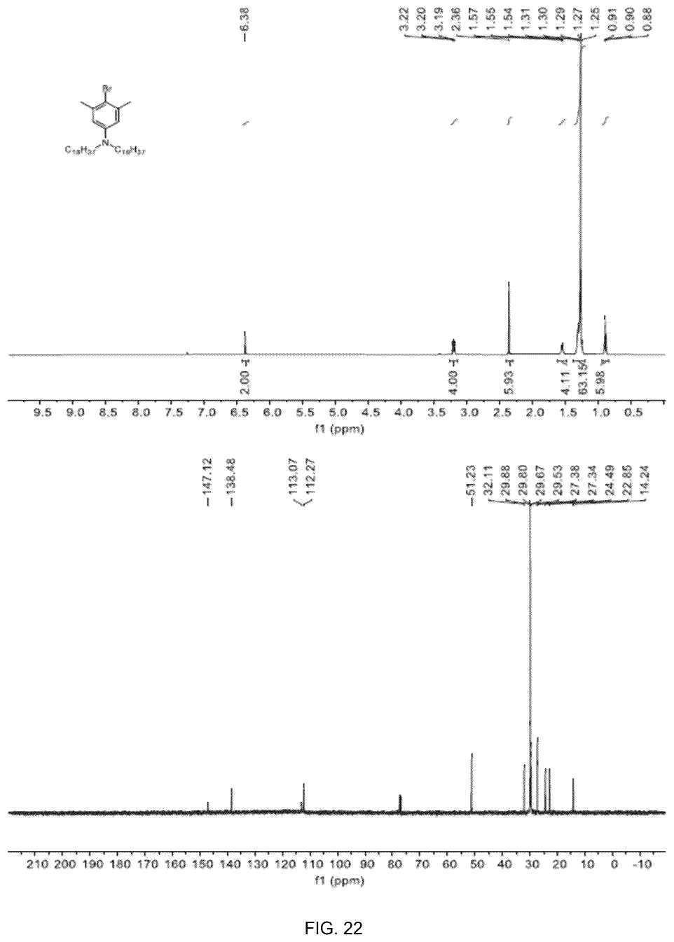

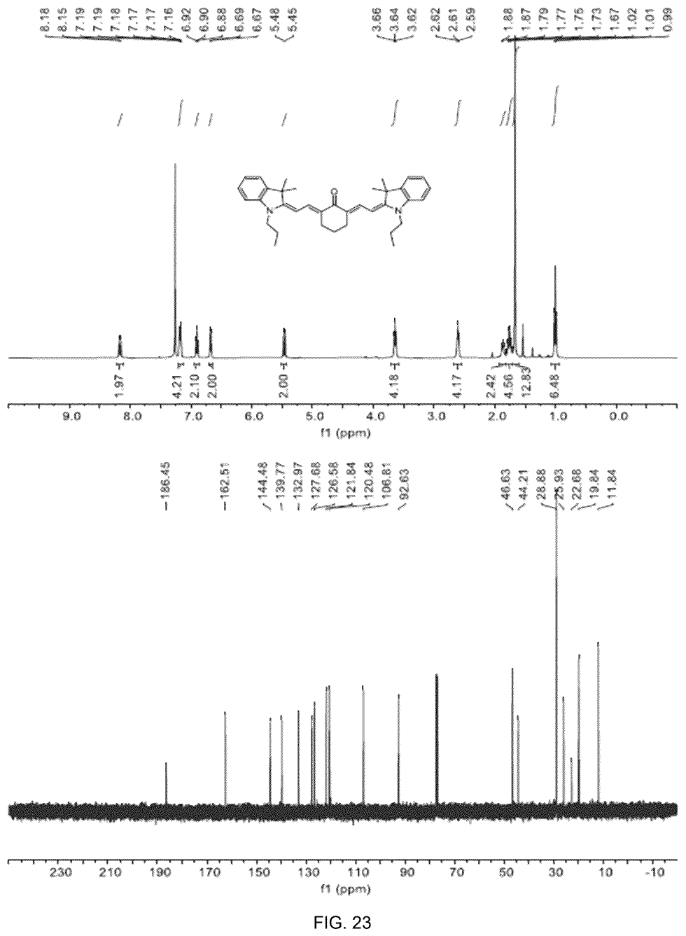

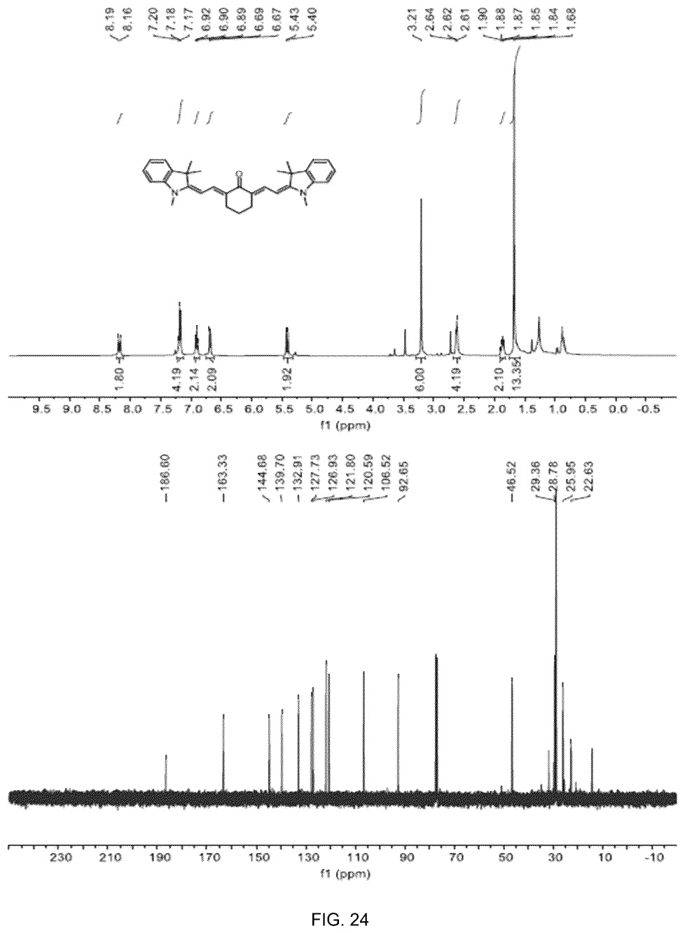

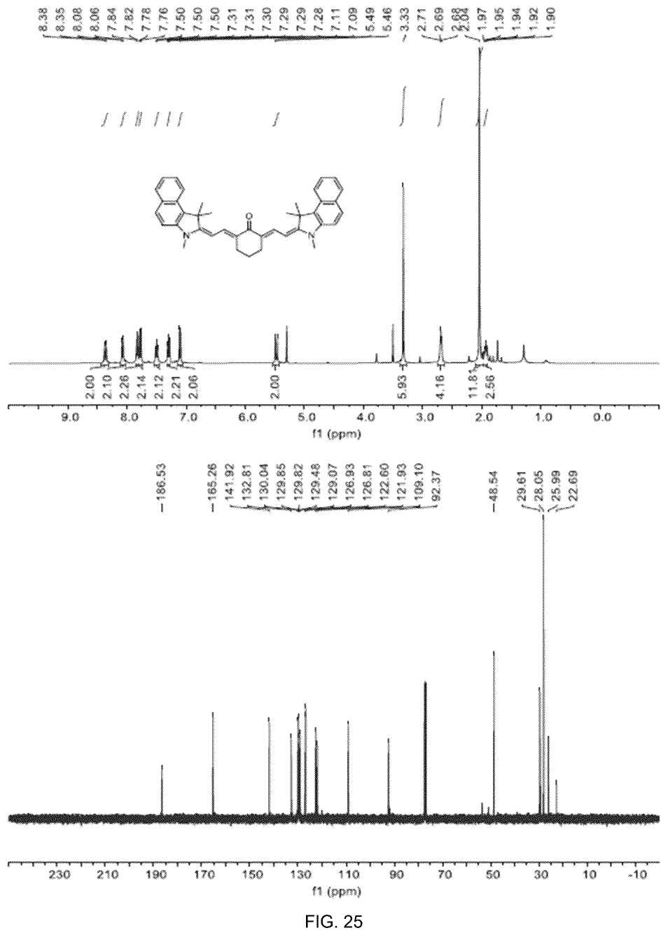

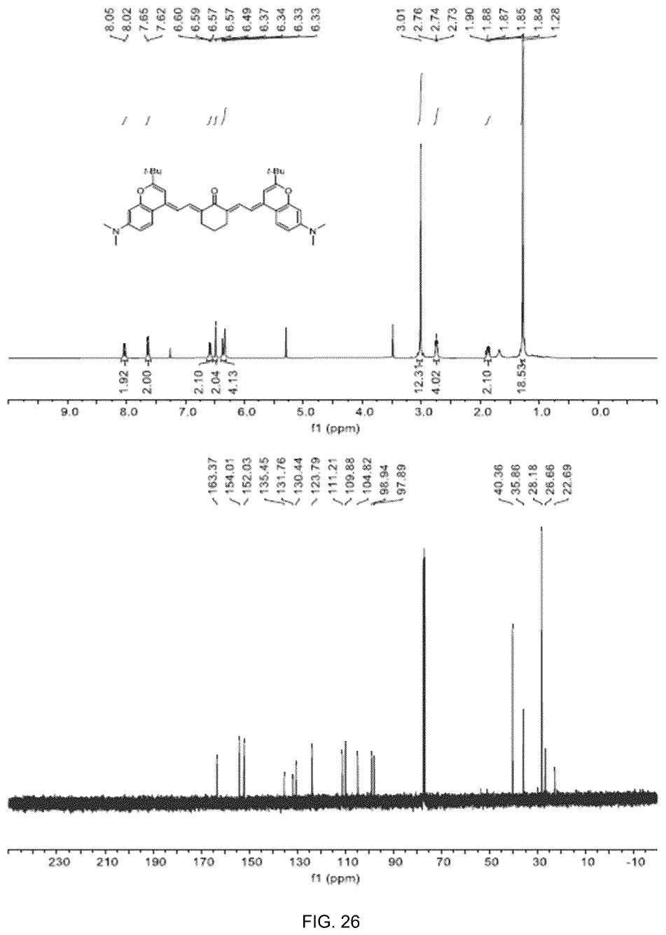

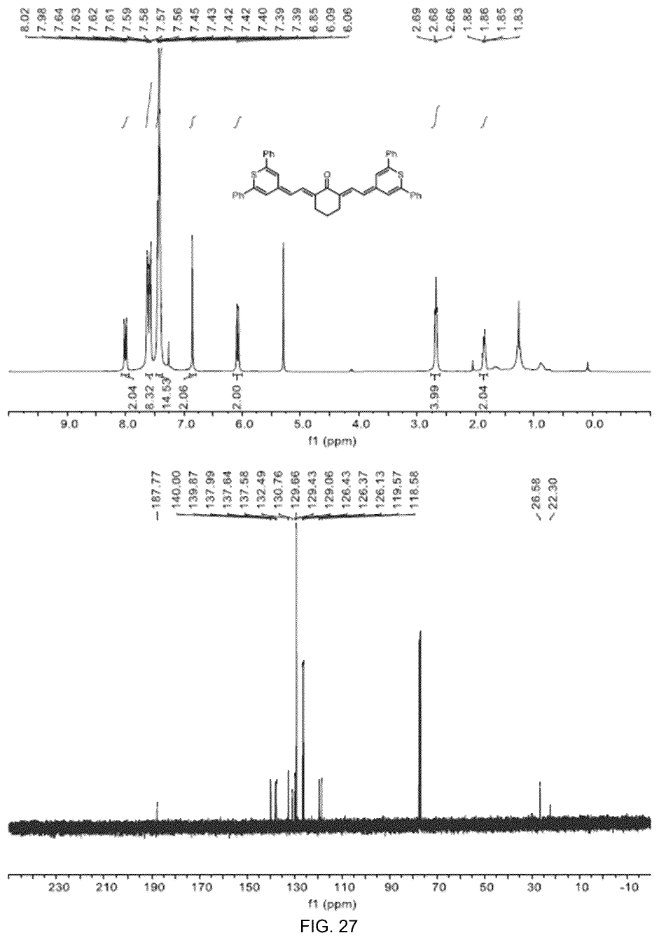

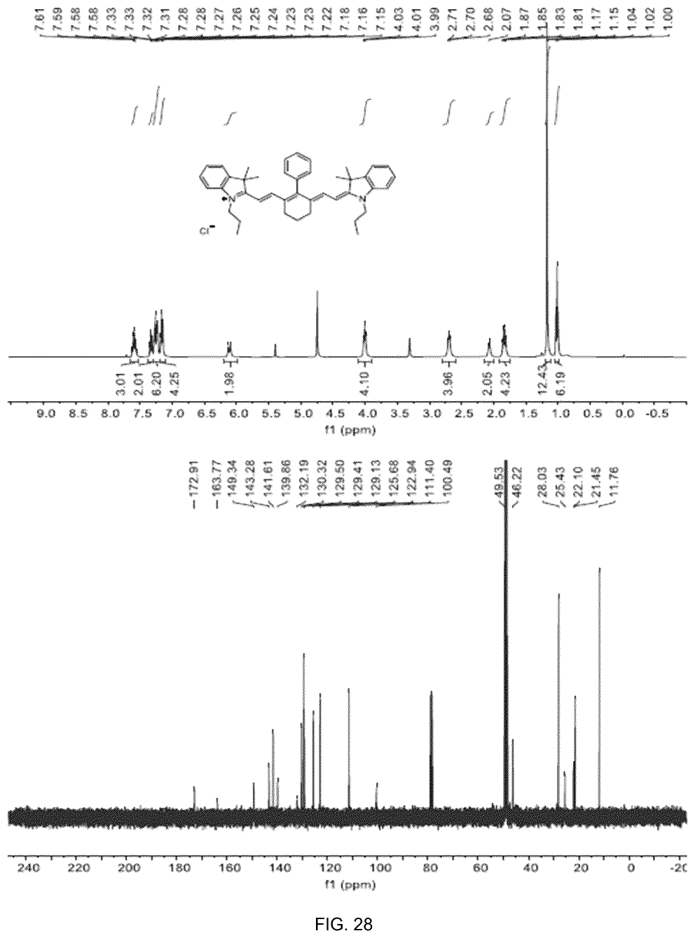

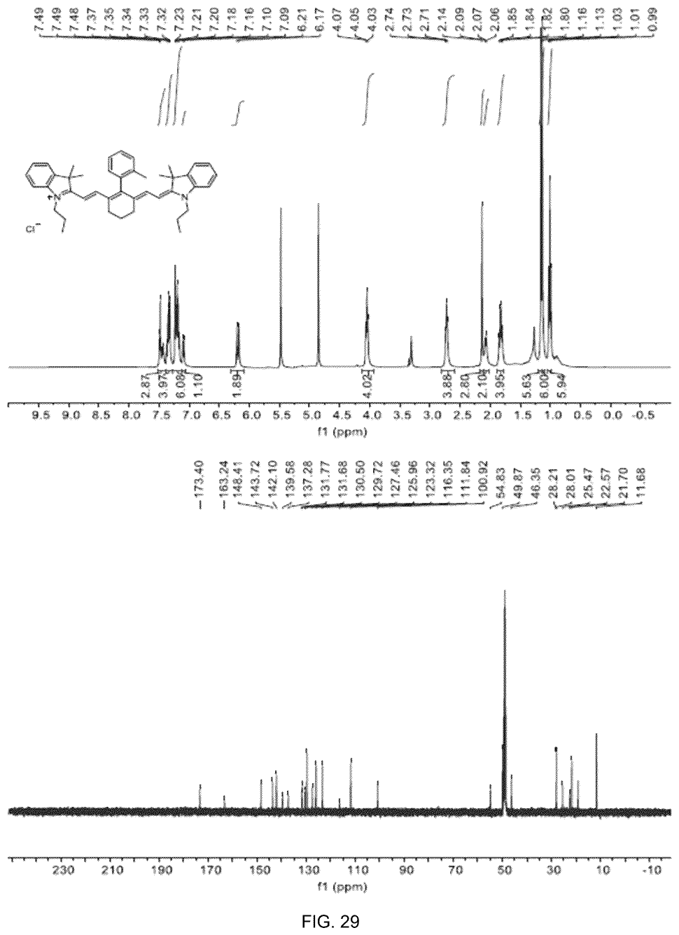

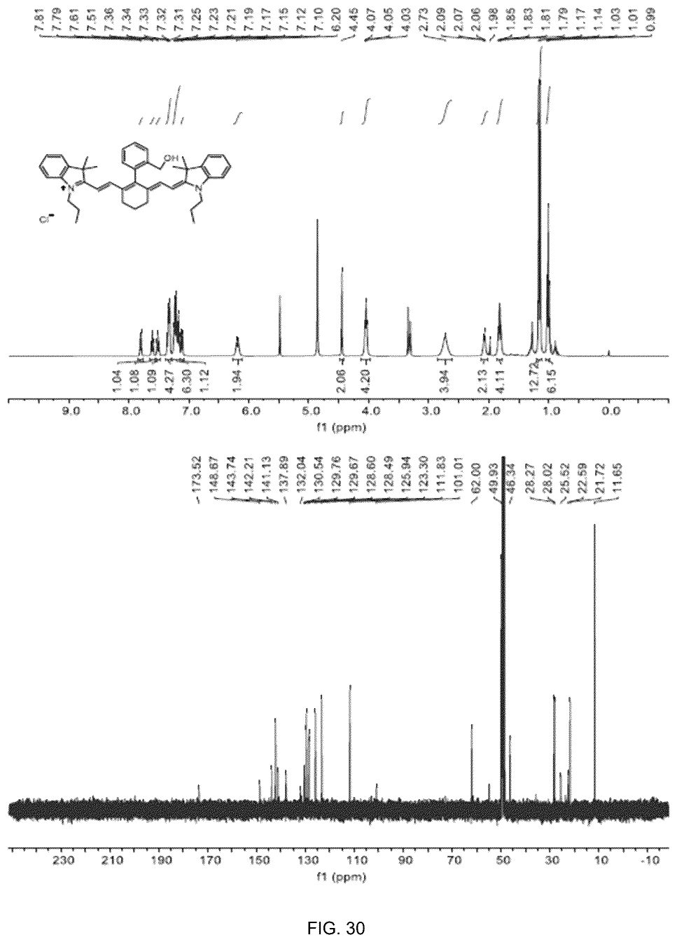

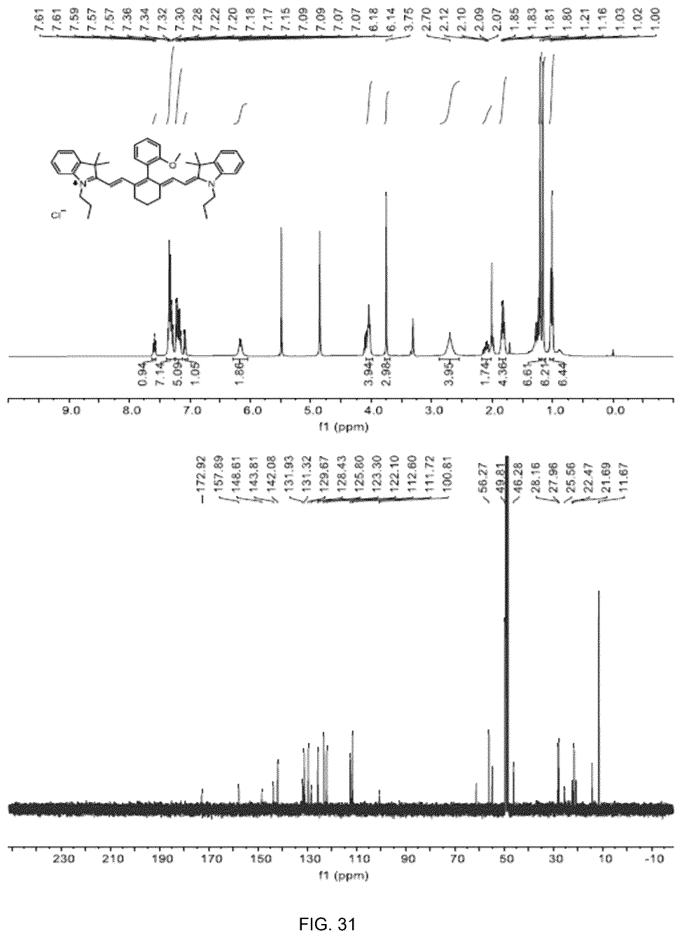

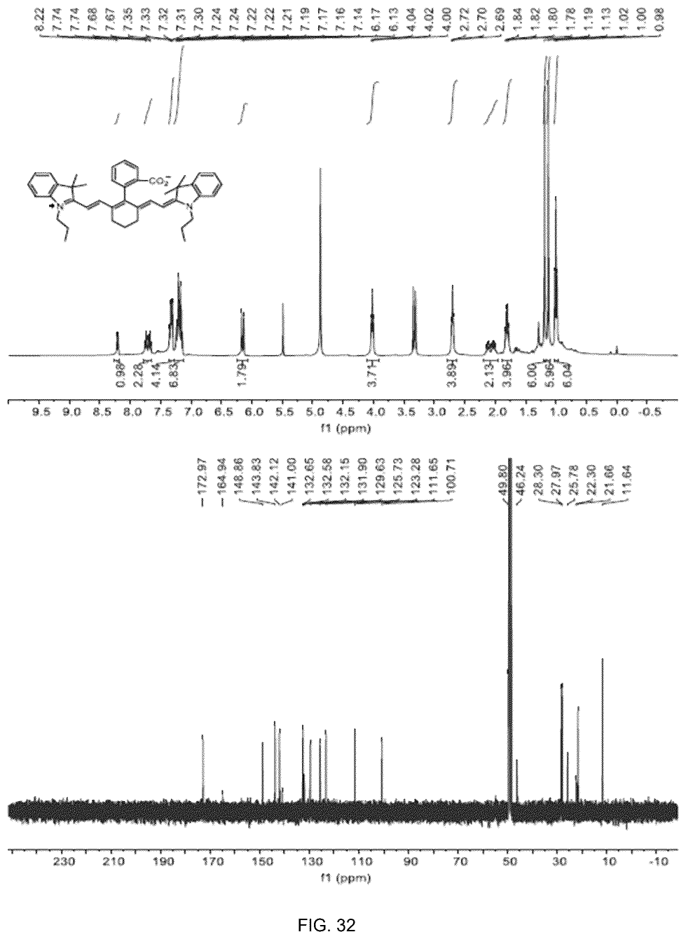

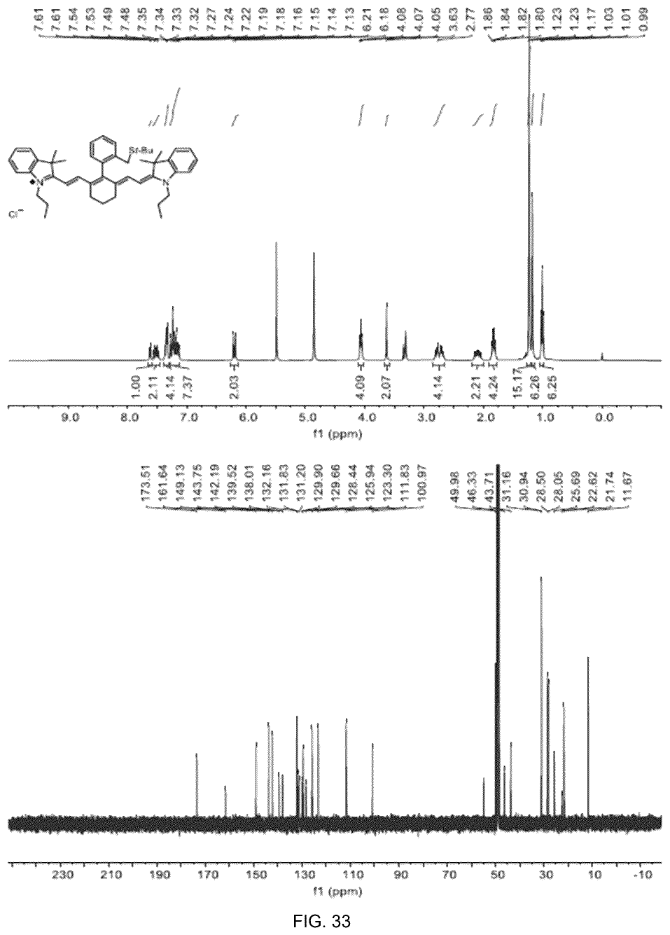

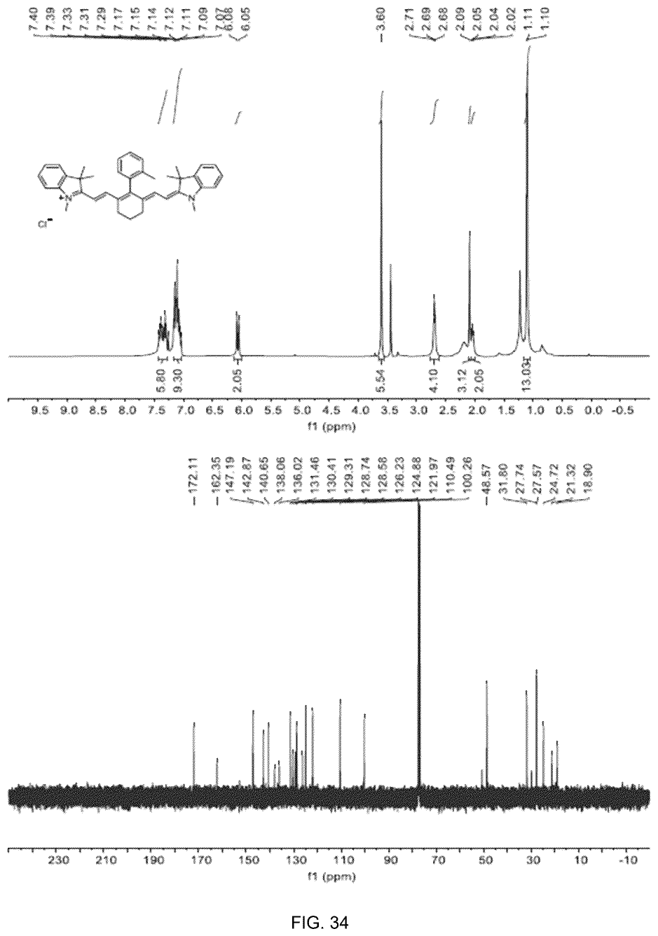

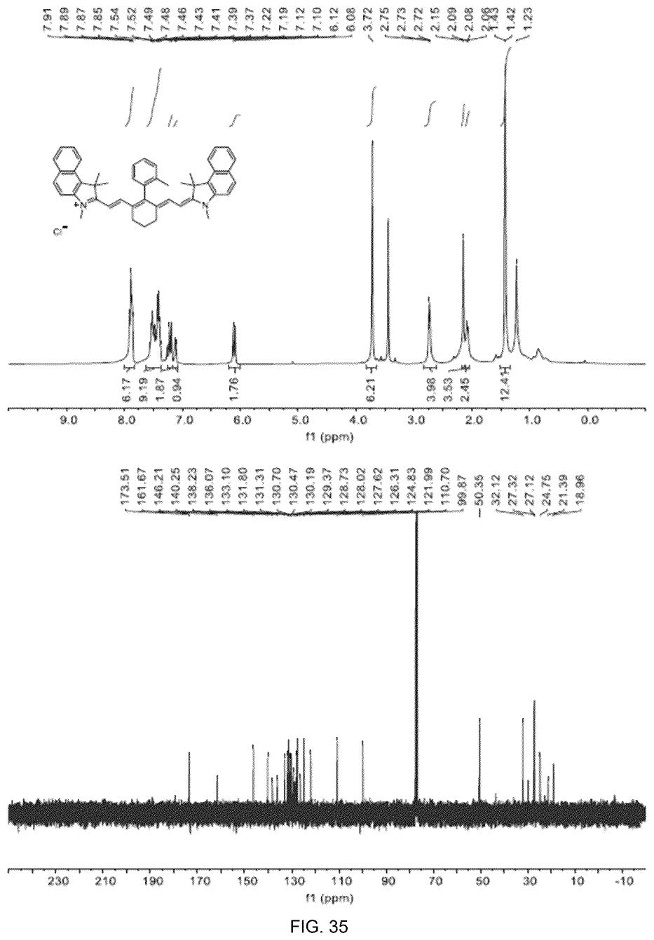

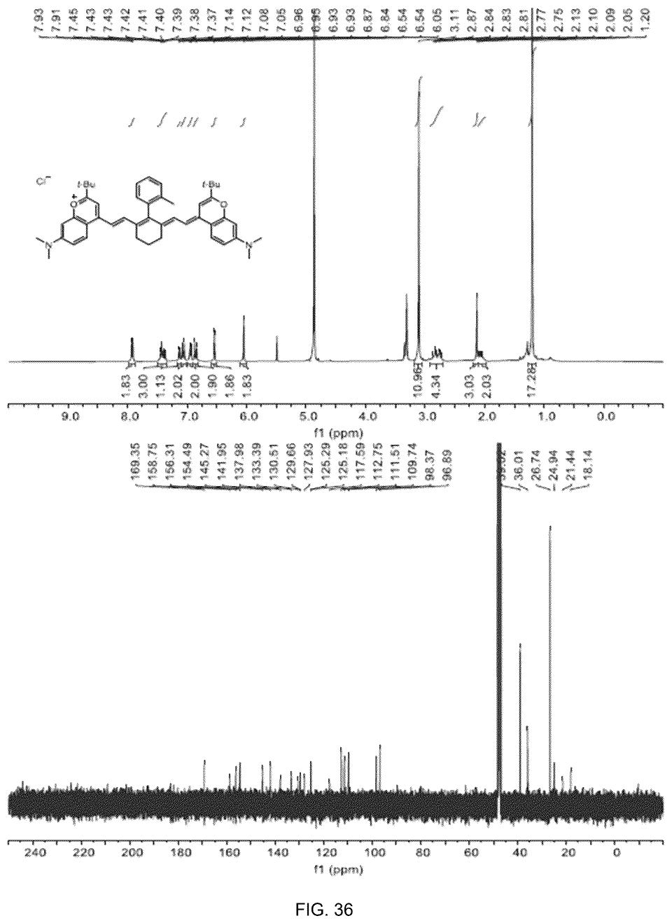

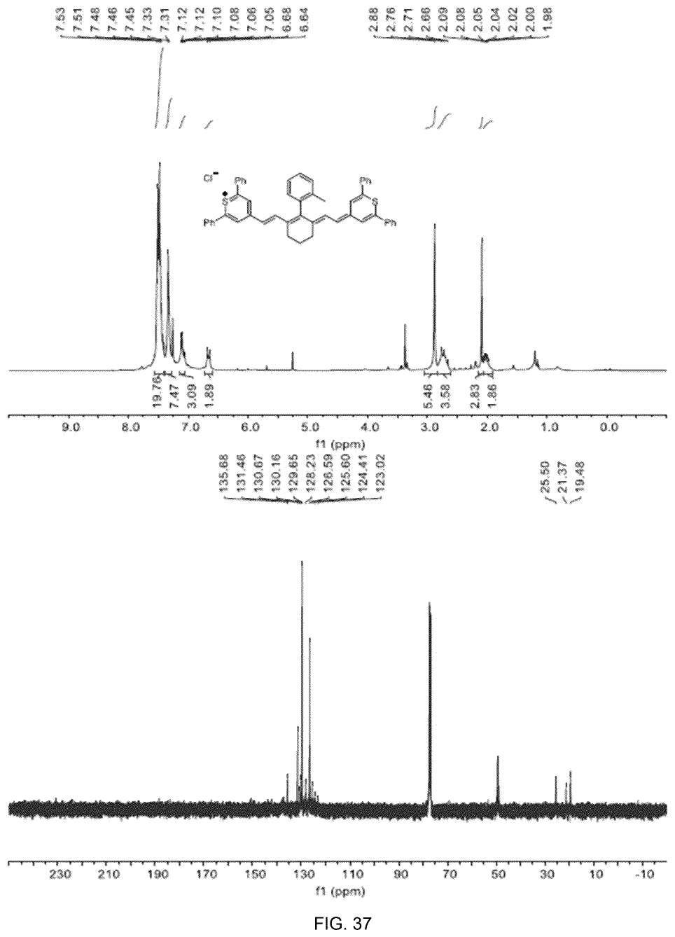

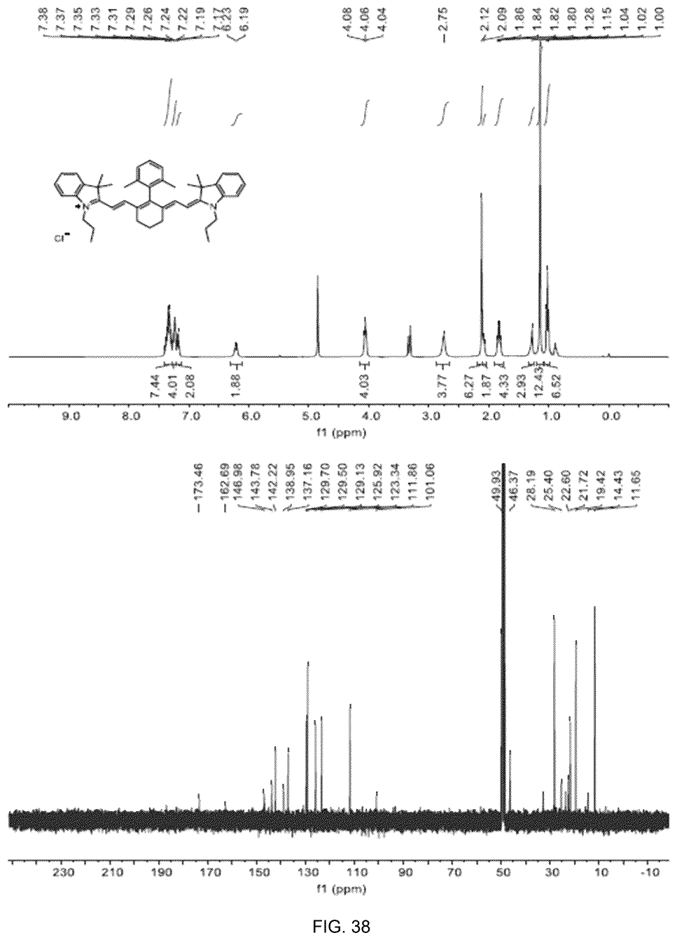

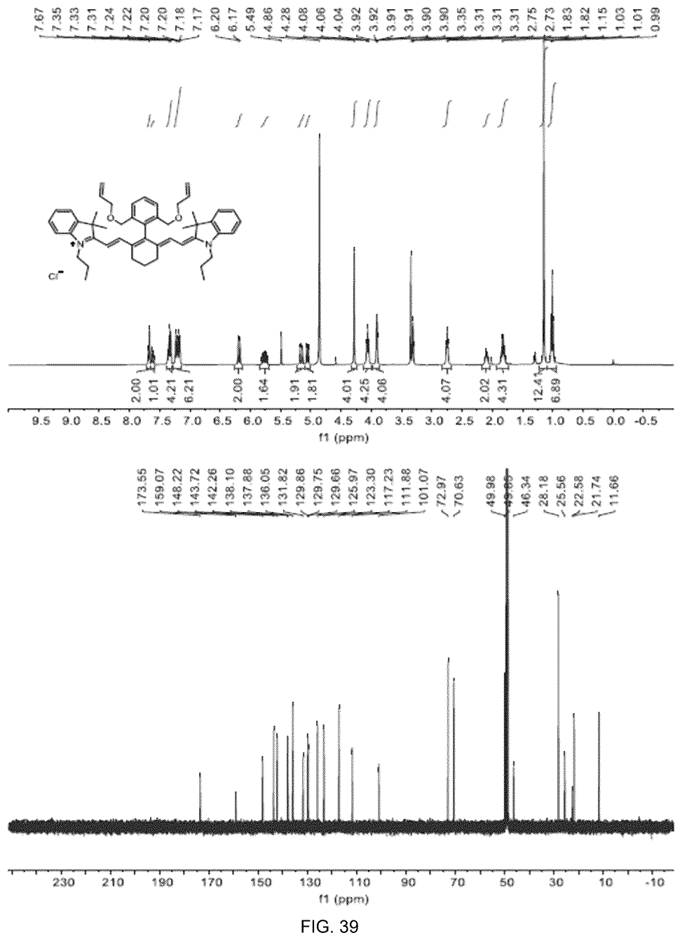

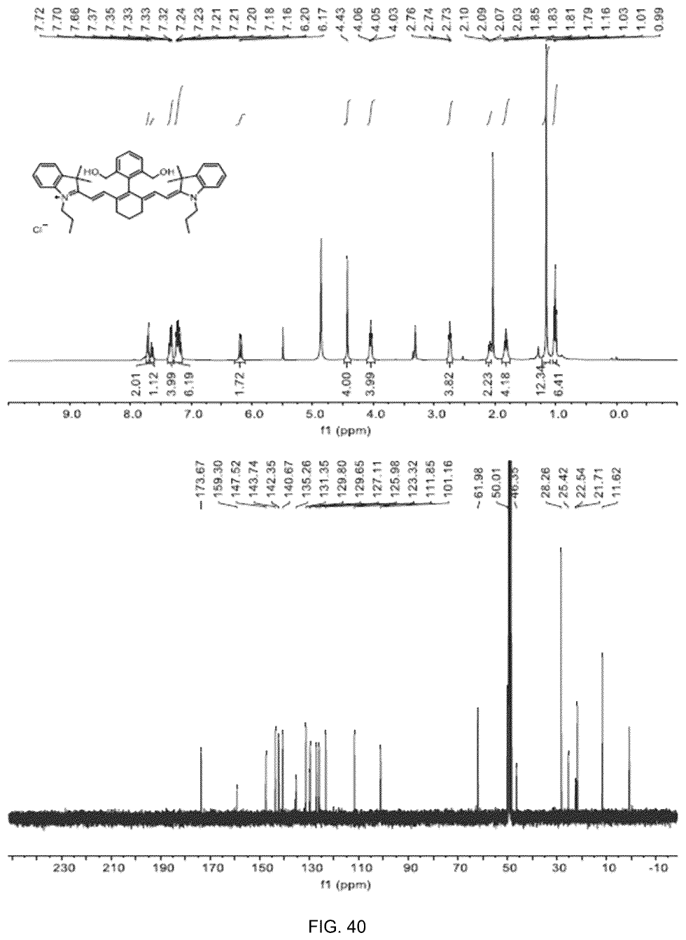

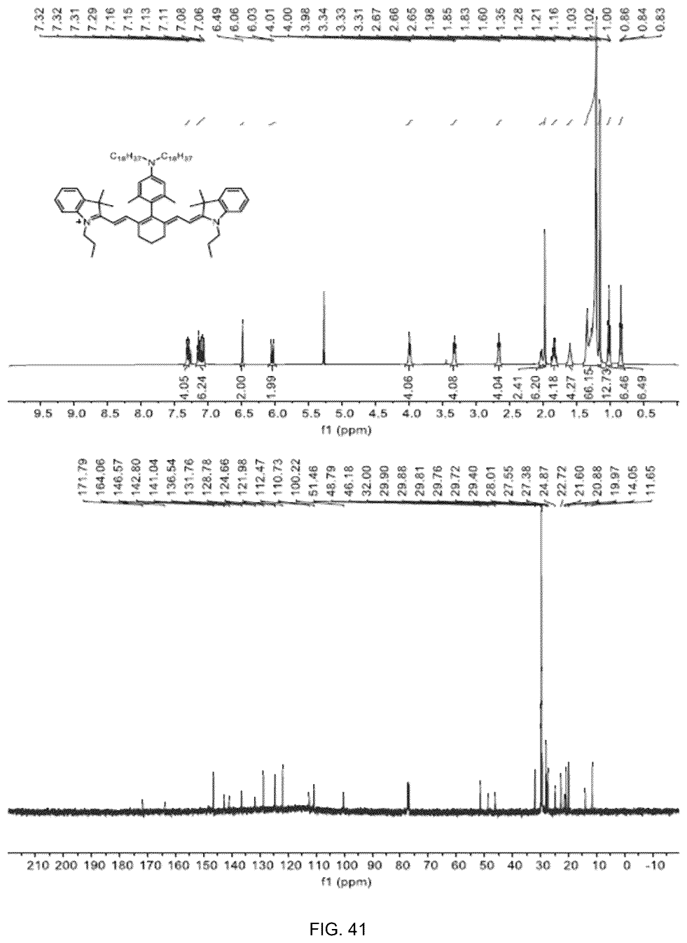

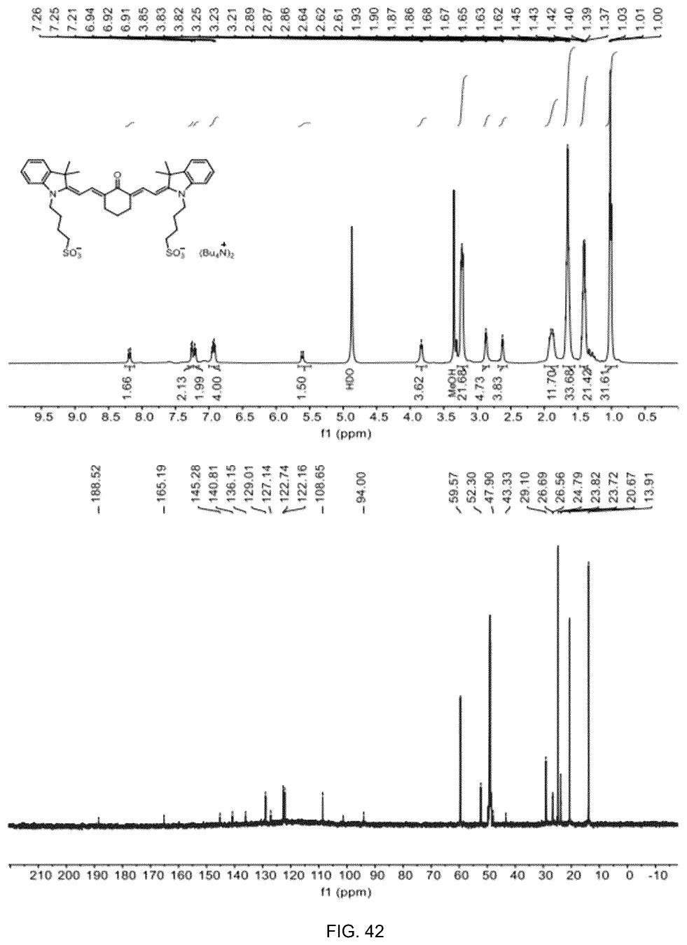

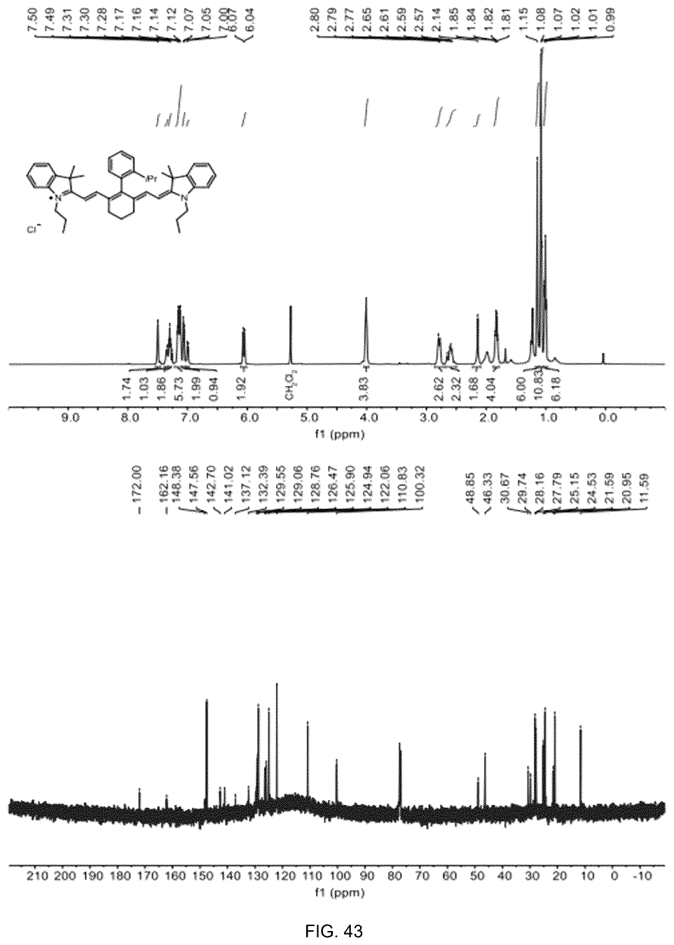

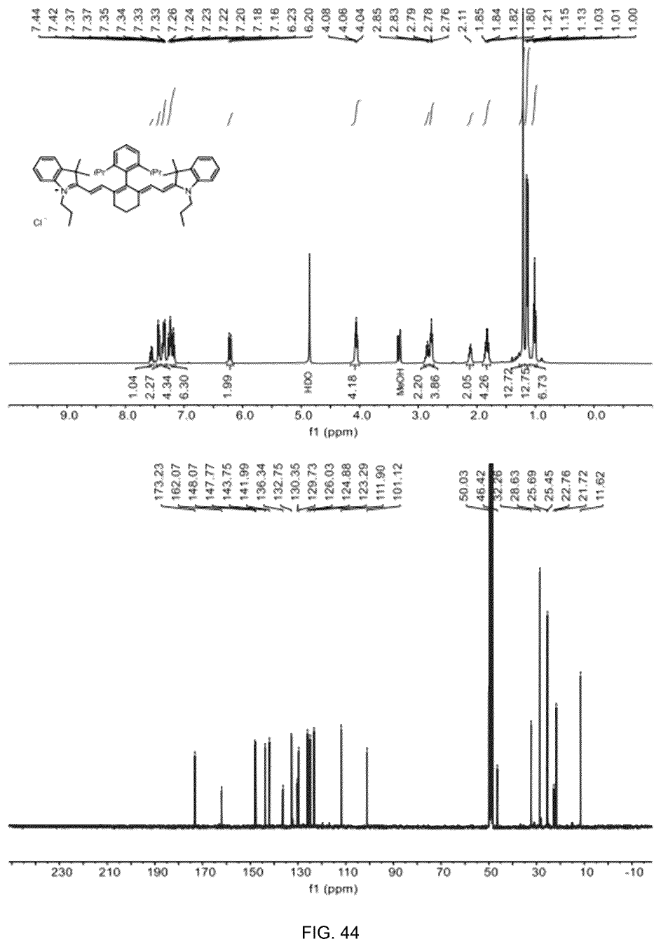

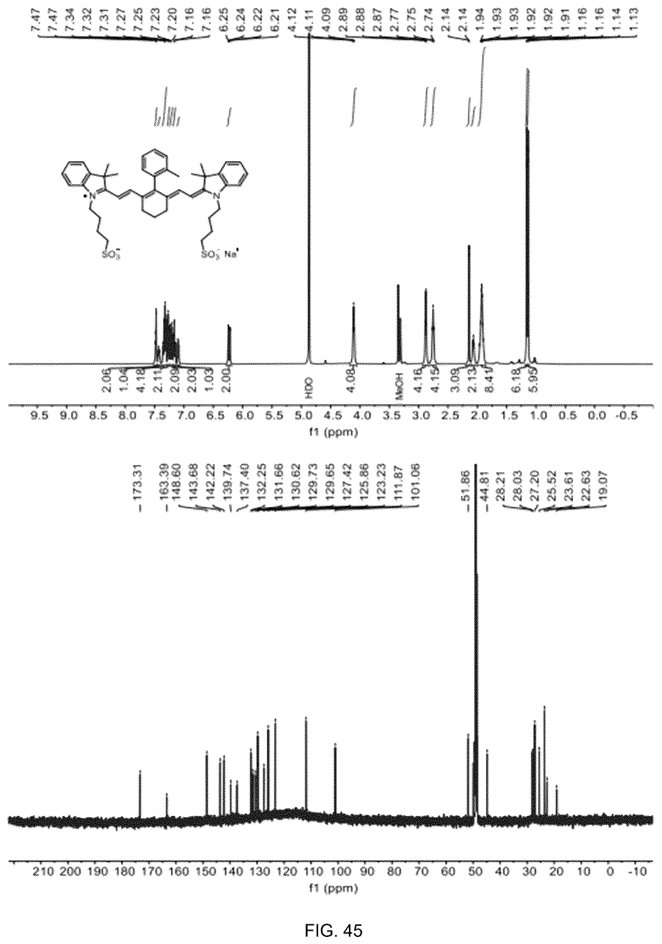

With reference to FIGS. 1-45, the preferred embodiments of the present invention may be described.

Synthesis development: We are interested in the development of this new polymethine dye synthesis because the building blocks have been already studied, which can be used as modular building blocks to create new fluorophores with desired properties. From the xanthene dye community, a variety of aryllithium reagents have been previously prepared from respective arylbromide to prepare xanthene fluorophores in diverse imaging and sensing requirements. On the other hand, polymethine chemists have made copious examples of heptamethine dyes with 4′-chloro substitution spanning wavelengths from NIR to SWIR. Additionally, a few 4′-chloro heptamethine dyes have been conveniently converted to the keto-form via S_{RN}1reaction in the presence of sodium acetate or N-hydroxysuccinimide. Against this backdrop, relating these two building blocks to the creation of 4′-aryl substituted heptamethine dyes make it easier to correlate synthetic and design strategies between the xanthene fluorophores and the red-shifted polymethine scaffolds.

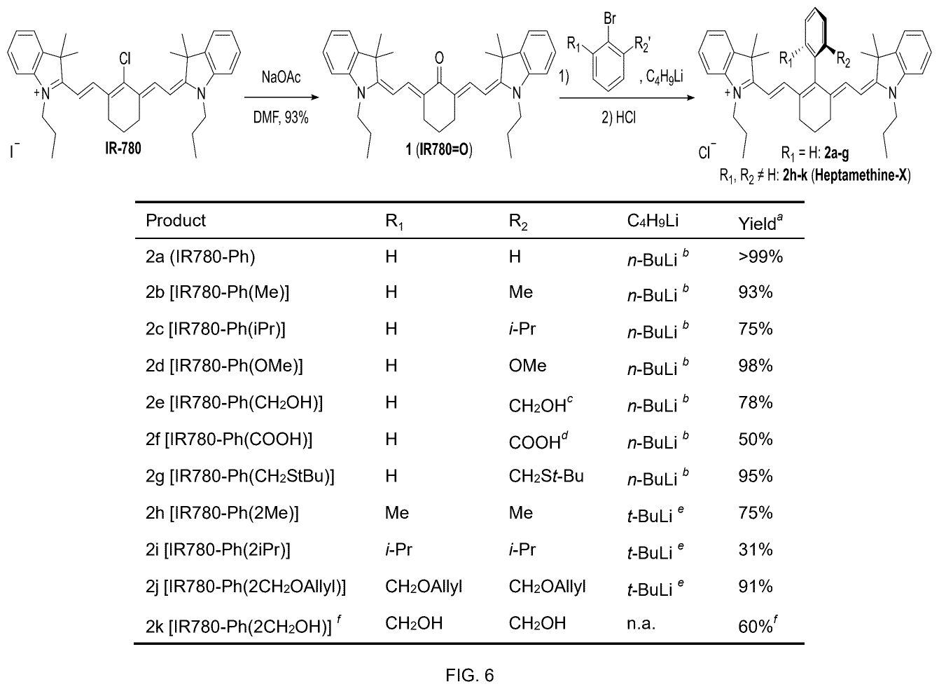

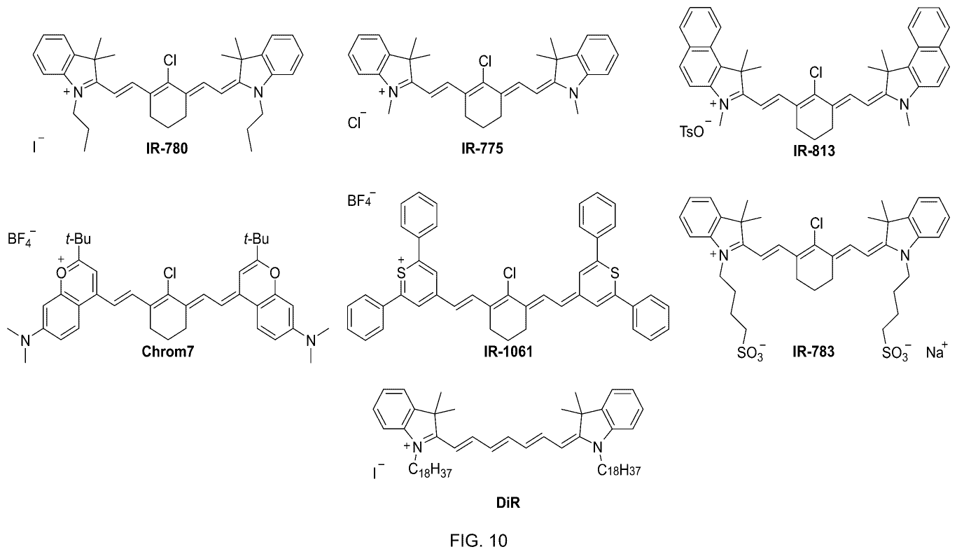

We selected IR-780 (FIG. 10) as our initial substrate, as this heptamethine dye is commercially available with low cost. The conversion of IR-780 to the keto-form 1 (IR780═O) using sodium acetate proceeded smoothly with a 93% yield. With this substrate, we tested the scope of the reaction with arylbromide reagents found in reported xanthene dye synthesis, and the results are summarized in FIG. 6. We started with bromobenzene, the simplest substitution, and found out that the conversion to 2a (IR780-Ph) was near quantitative. Notably, the reaction condition employed here was generic for the addition of lithium-halogen-exchanged intermediate to the xanthone derivatives without optimization. We then introduced ortho-substituents to the aryl group, a frequently used structure in the xanthene dyes. We started with 2-bromotulene, which was used in the synthesis of xanthene dyes such as TokyoGreen and TokyoMagenta, and as expected, the reaction afforded 2b [IR780-Ph(Me)] also with high yield (93%). Reaction to introduce larger inert functionalities at the ortho-position of central phenyl substitution, including bulky isopropyl in 2c [IR780-Ph(iPr)] and methoxy in 2d [IR780-Ph(OMe)] also succeeded, with good to excellent yields. We then turned to aryl substitutions with ortho-nucleophilic functionalities, as this has been widely explored in the xanthene dye designs for on/off modulation of the fluorophore. With nucleophilic groups protected in the aryl bromide, we were able to synthesize 2e [IR780-Ph(CH₂OH)] using trimethylsilyl ether as protection for the hydroxyl and 2f [IR780-Ph(COOH)] using tert-butyl ester as protection for the carboxyl group, both resulted in appreciable yields. To note, these protection groups do not need an additional deprotection step, likely due to the help from the newly-formed alkoxide prior to acid quenching. The reaction is also compatible with the aryl bromide containing tert-butyl thioether group to furnish 2g [IR780-Ph(CH₂StBu)], a precursor towards the thiol-containing heptamethine dye.

Encouraged by the mild condition and high yields of our modification method, we then seek to introduce steric bulk to the 4′-position of Cy7, as the increasing steric demand has been reported to not only discourage undesired fluorophore aggregation but also enhance the stability of the molecule. Against this backdrop, we first used 2-bromo-1,3-dimethylbenzene as a simple substrate. To compensate for the increased sterics demand, we used tert-butyllithium as a more reactive reagent while keeping the rest of the procedure the same. Satisfyingly, the reaction afforded 2h [IR780-Ph(2Me)] with a 75% yield. Taking a step further, we used 2-bromo-1,3-diisopropylbenzene as the substrate, with considerable steric bulk at the reaction site that is challenging even for canonical synthesis of xanthene dyes. Although part of the reactants underwent uncharacterized isomerization affording a blue-shifted heptamethine side product, the reaction still furnished 2i [IR780-Ph(2iPr)] although with a lower yield of 31%. Additionally, to incorporate both steric bulk and hydrophilicity to the fluorophore scaffold, we introduced two hydroxymethyl groups as ortho-substitutions on the 4′-phenyl to introduce both steric bulk and hydrophilicity to the fluorophore. The addition reaction proceeded smoothly to give 2j [IR780-Ph(2CH₂OAllyl)] with allyl ether as the protection group of the hydroxyls. Further deprotection of the allyl ether by palladium catalysis provided 2k [IR780-Ph(2CH₂OH)] with two free hydroxyl groups around the methine bridge, with an overall yield of 60%. Owing to their Xanthene dye-inspired synthesis and the “X” shape of the molecule when viewed along the 4′ C—C bond, we coined the name “heptamethine-X” for heptamethine fluorophores 2h-2k with 4′ aryl substitution carrying two ortho-functionalities. Collectively, these successes highlight the merit of our method in the synthesis of challenging polymethine fluorophores using existing and new aryl bromide building blocks.

Remarkably, several of these modifications, especially the make of heptamethine-X scaffold, are previously not accessible from conventional Suzuki coupling conditions on 4′-chloro Cy7 scaffolds. Specifically, the introduction of ortho-unsubstituted or mono-substituted aryl moieties included in 2a and 2e, by Suzuki coupling with corresponding boronic acids generally requires prolonged heating (several hours to days) at high temperature (≥90° C.) with modest yields. Due to the increasing steric demand, the Suzuki coupling of ortho-methyl-substituted phenyl was only reported on the Schiff base prior to the fluorophore synthesis with even harsher conditions, and the ortho-disubstituted counterpart was only reported using bottom-up synthesis from customized Zincke salt. These difficulties showcase the advantage of our mild and fast pathway for the introduction of 4′-aryl substitutions to existing heptamethine fluorophore scaffolds without rebuilding the fluorophore core.

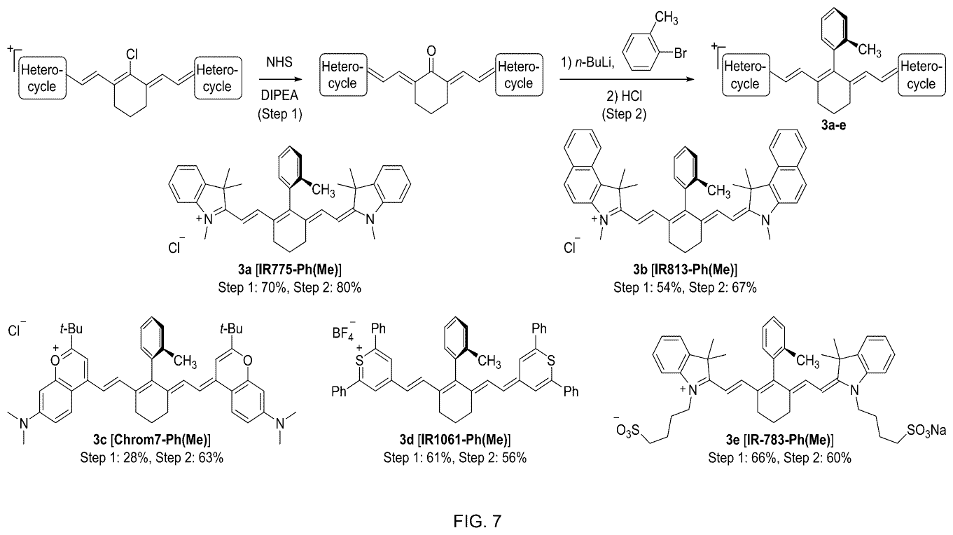

We followed by testing the compatibility of our methods on heptamethine fluorophores with other heterocycles (FIG. 7). We began with IR-775, another Cy7 dye with dimethyl substitutions in indolium nitrogen (FIG. 10). The lack of longer aliphatic chains in IR-775 makes this compound less soluble in organic solvents; while the hydrolysis reaction towards keto-intermediate with sodium acetate was possible, the separation of the product mixture was difficult due to the limited solubility in organic solvent. As such, we opted for the hydrolysis using N-hydroxysuccinimide to achieve cleaner products. Despite these complications, the aryllithium addition to prepare 3a [IR775-Ph(Me)] in the second step readily proceeded with 80% yield. We then moved to heptame-thine dyes with longer working wavelength, including NIR dye IR-813 and SWIR-emitters Chrom7 and IR-1061 (FIG. 10). Although these extended conjugated structures exhibit lower solubility in organic solvents, their synthesis was carried out successfully without the need to adapt reaction conditions to afford 3b-d. Lastly, to showcase the compatibility with heptamethine dyes carrying multiple sulfonate groups, a widespread functionality for introducing hydrophilicity to hydrophobic fluorophores, we picked IR-783 as a substrate. Although more soluble in water, molecules with sulfonate groups are generally insoluble in common organic solvents. To circumvent this issue, we exchanged sodium ion into tetrabutylammonium ion for the intermediate during its purification on HPLC, affording the keto-form of IR-783 freely soluble in THF during the second step. With this modification, we employed our general synthetic methods and successfully obtained 3e [IR783-Ph(Me)]. Taken together, these results demonstrate the versatility of our method to introduce aryl substitutions at the 4′-position of heptamethine dyes, enabling further tuning of the property of the fluorophore for various imaging applications.

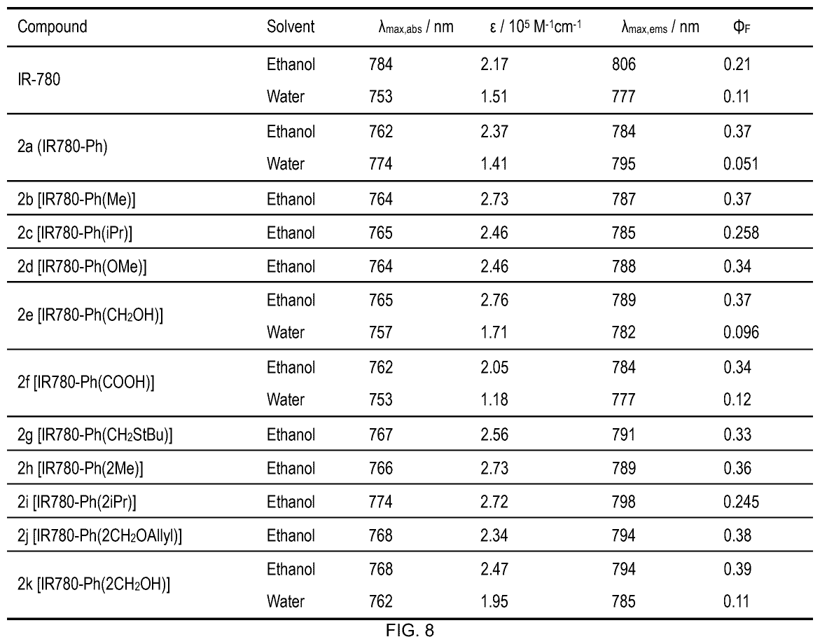

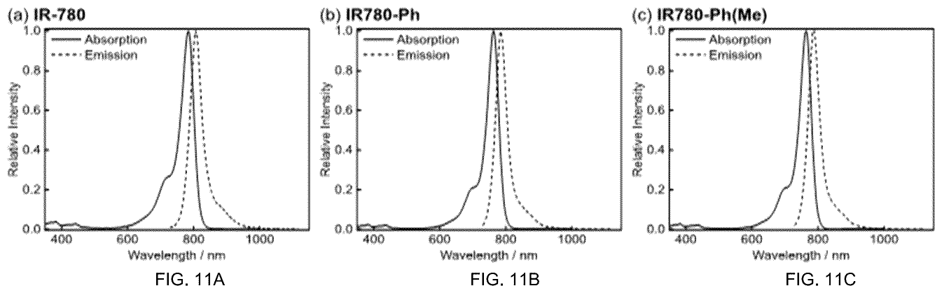

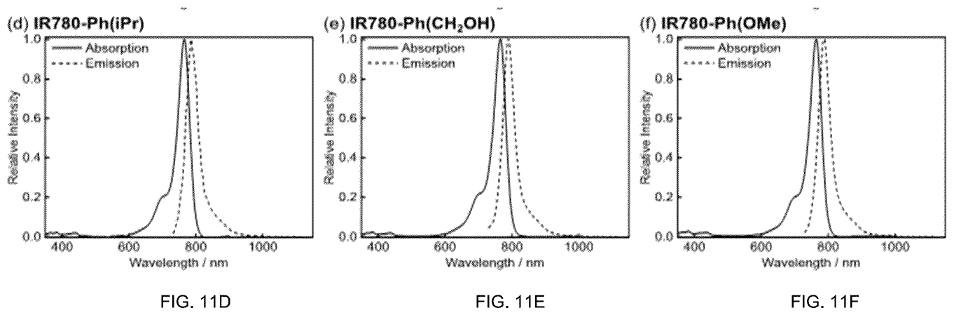

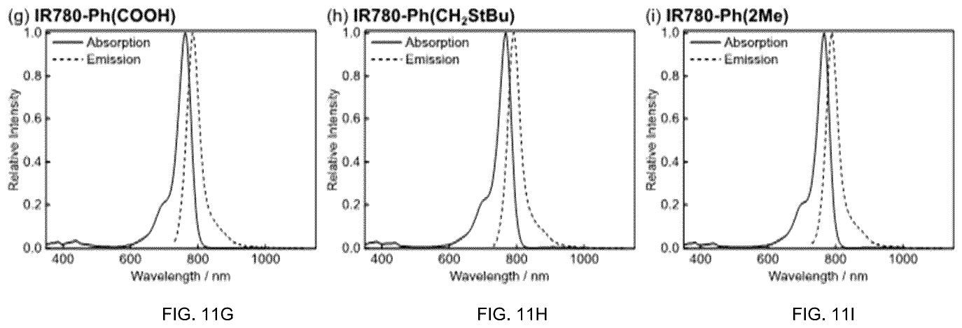

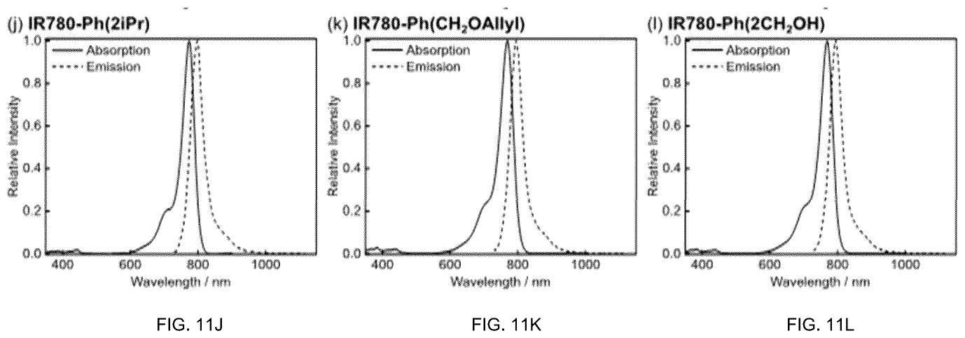

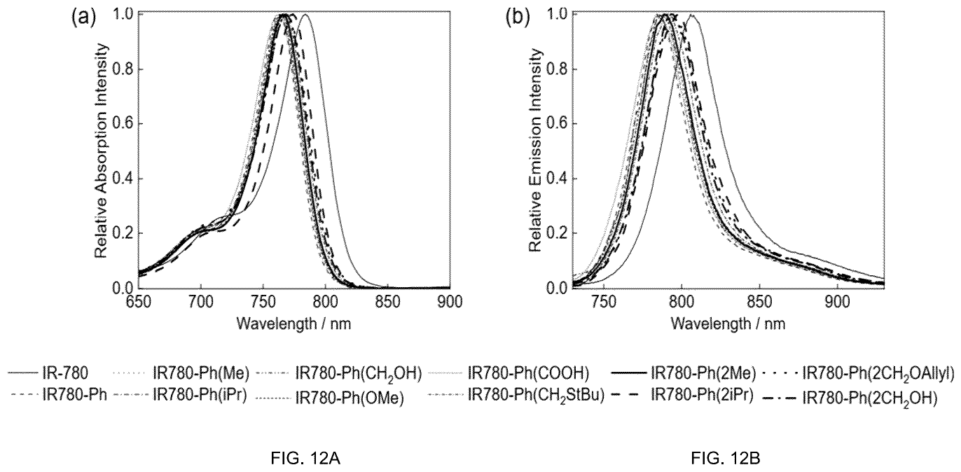

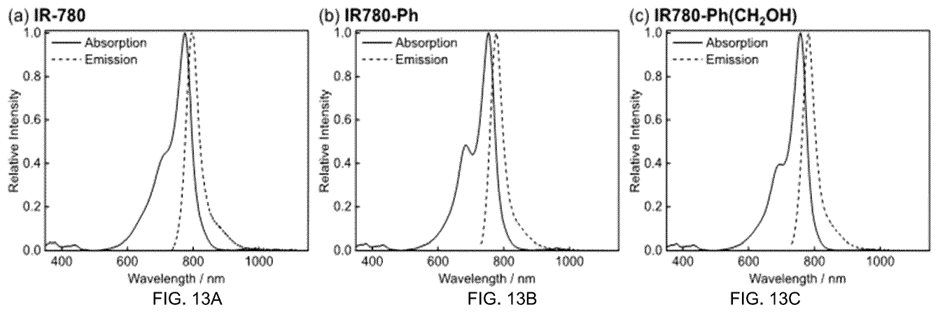

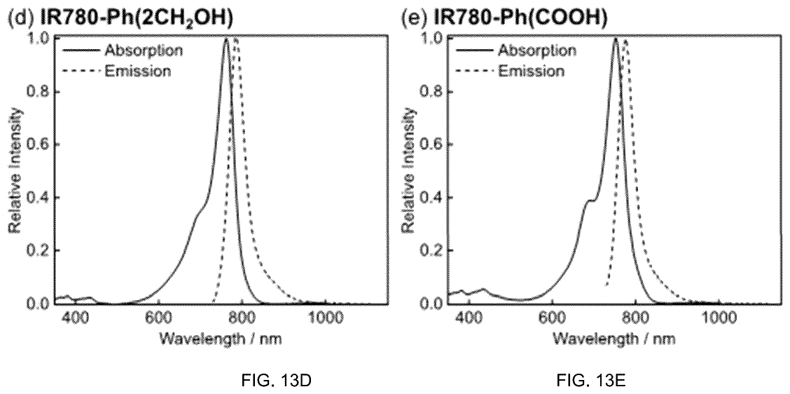

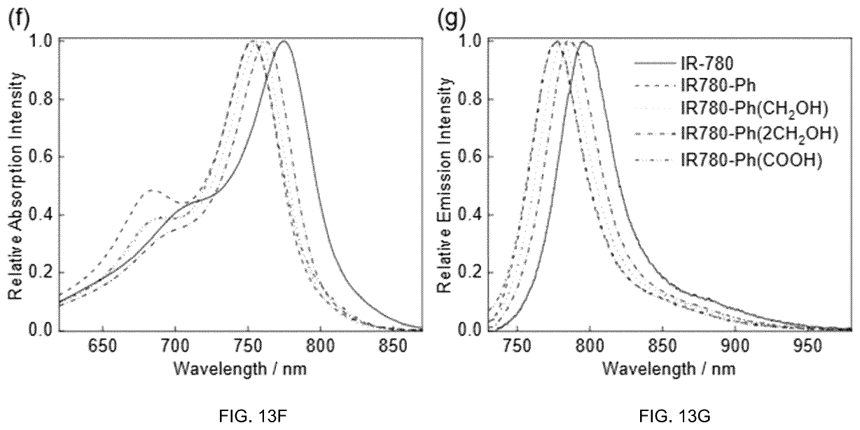

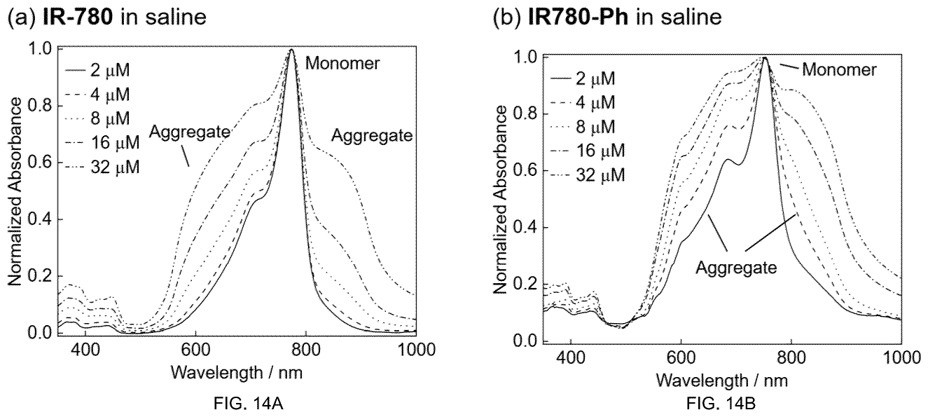

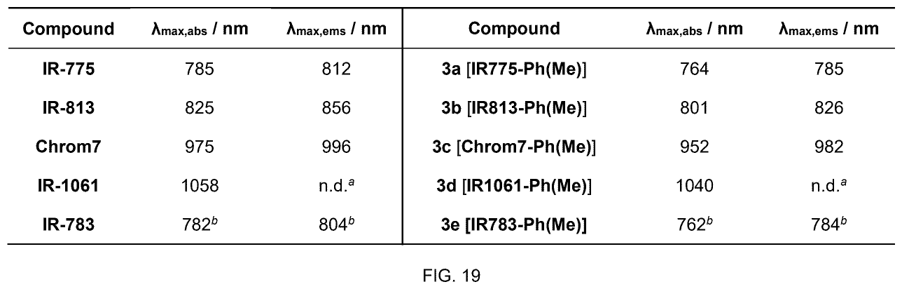

Improvement of heptamethine fluorophores by 4′-aryl substitution: With the fluorophores in hand, we further tested their photophysical properties and compared them with the parent fluorophore. As summarized in FIG. 8, all newly synthesized IR-780 derivatives show absorption maxima within a narrow range of 762-768 nm and emission maxima in 784-795 nm in ethanol (FIGS. 11A-12B), which confirms that the 4′-aryl substituents are not coplanar with the fluorophore core, participating little in the conjugation system of the fluorophore. These wavelengths are slightly blue-shifted compared to parent IR-780 due to the removal of conjugated chlorine atom. Similarly, modification with ortho-methylphenyl on other heptamethine scaffolds results in slight blueshift of the emission and excitation wavelengths (FIG. 19). These IR-780 derivatives show high absorption coefficients (>2×10⁵ M⁻¹cm⁻¹ in ethanol), which is characteristic of polymethine fluorophores. Additionally, by replacing the chlorine atom in IR-780 with aryl substituents, these fluorophores exhibit 1.5 to 2-fold higher fluorescent quantum yields in ethanol as the starting IR-780, which is in agreement with previous observations of similar modifications. We also tested photophysical properties of some fluorophores in aqueous solutions and observed similar absorption coefficient and quantum yields with IR-780 (FIG. 8; FIG. 13A-G). The overall similar photo-physical behavior of the new derivatives suggest that aryl-modification at the 4′ position does not much affect the fluorophore core.

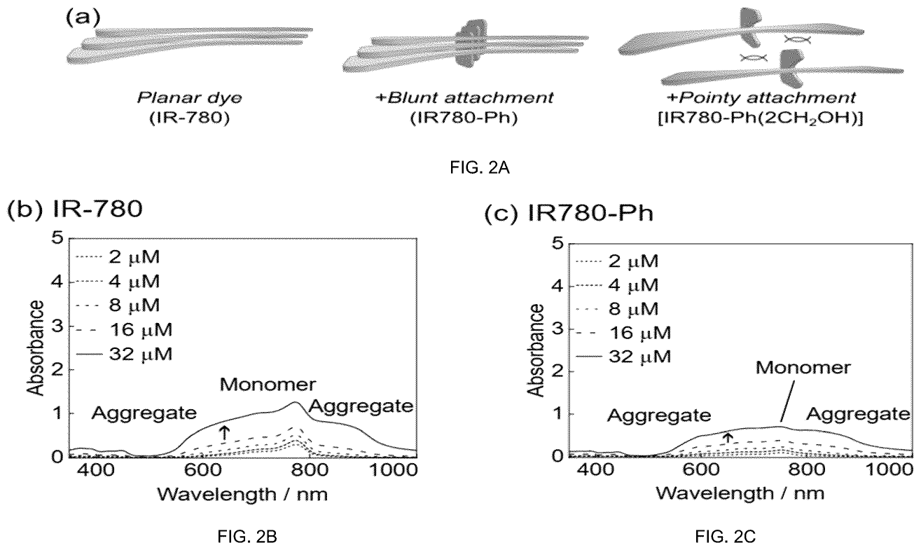

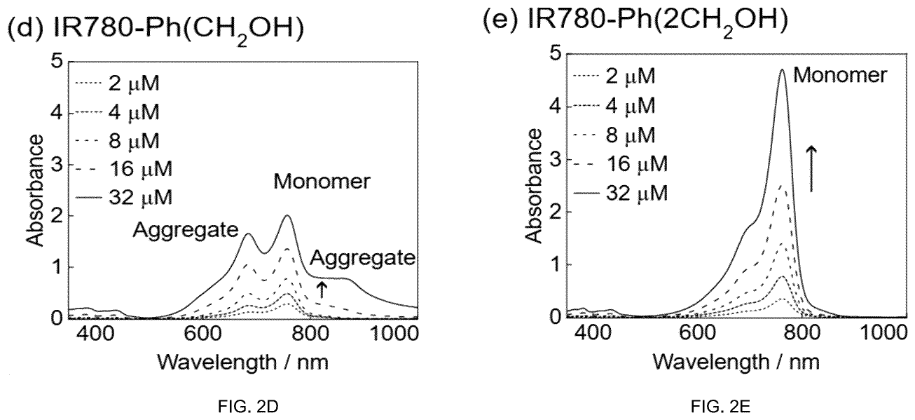

We then tried to verify the reduction of aggregation tendency for fluorophore carrying 4′-phenyl with ortho-disubstitutions (heptamethine-X) compared to the parent dye and its mono-substituted derivative. The perpendicular orientation of the 4′-aryl group positions its ortho-functionalities right over the polymethine bridge, which can discourage the close stacking between fluorophores to reduce the aggregation of the fluorophores (FIG. 2A). Previous reports have utilized Zincke salt carrying ortho-substituents to create “shielded” Cy7 derivatives for reduced aggregation, which requires lengthy synthesis. A simpler strategy uses Suzuki coupling on the Schiff base to access 4′-(o-methyl)phenyl substitution on a SWIR-emitting heptamethine, showing greatly reduced aggregation behavior compared with analog without ortho-methyl group; however, the difficulty of synthesizing ortho-disubstituted derivative prevents further exploration of this effect.

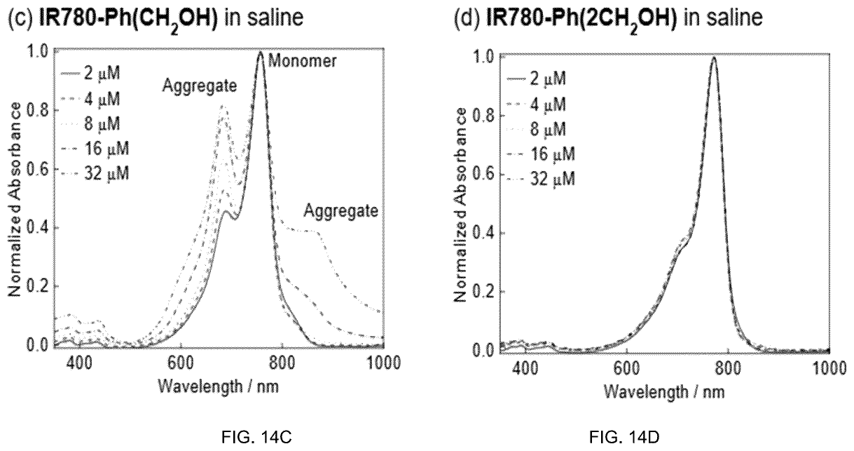

Against this back drop, we selected IR780-Ph, IR780-Ph(CH₂OH) and IR780-Ph(2CH₂OH) for water solubility comparison against IR-780. Since all the dyes are able to form monomeric absorption in dilute solutions in water (FIG. 13A-G), we selected PBS as the solvent, where the existence of saline can induce the aggregation of the hep-tamethine dyes even with multiple hydrophilic function-alities. We measured the absorption profile of each dye with increasing concentration to determine the monomer population in solution (FIGS. 2B-E; FIG. 13). While IR-780 is able to dissolve in PBS at 2 μM and show primarily monomer absorption, the aggregated population, both red-shifted and blue-shifted, increases dramatically as the concentration increases, resulting in an absorption spectrum largely deviated from monomer cyanine absorption profiles at 32 μM (FIG. 2B). The aggregation propensity of IR780-Ph is even larger (FIG. 2C), as the hydrophobic phenyl group can also form stacking perpendicular to the dye plane and thus promotes aggregation (FIG. 2A). In contrast, with the introduction of hydroxymethyl as the ortho-substituent in IR780-Ph(CH₂OH), which brings in steric bulk and hydrophilicity to one side of the molecule, the monomeric absorption becomes more dominant at higher concentrations with a more defined blue-shifted absorption peak, likely from H-dimers (FIG. 2D). With hydroxymethyl groups on two sides in IR780-(2CH₂OH), this solubilization effect is greatly enhanced, affording a cyanine-like absorption profile almost independent of concentration (FIG. 2E), reaching an absorbance of 4.7 at 32 μM with 1 cm light path, which is almost four-fold compared to IR-780 at the same concentration (FIGS. 14A, 14D). This strong reduction in aggregation in IR780-(2CH₂OH) demonstrates the necessity of introducing the steric bulk on both sides of the fluorophore, and thus the introduction of 4′-aryl substitution carrying ortho-dihydroxymethyl functionality in our heptamethine-X scaffold provides a facile choice for modifying existing fluorophores towards aggregation reduction in water.

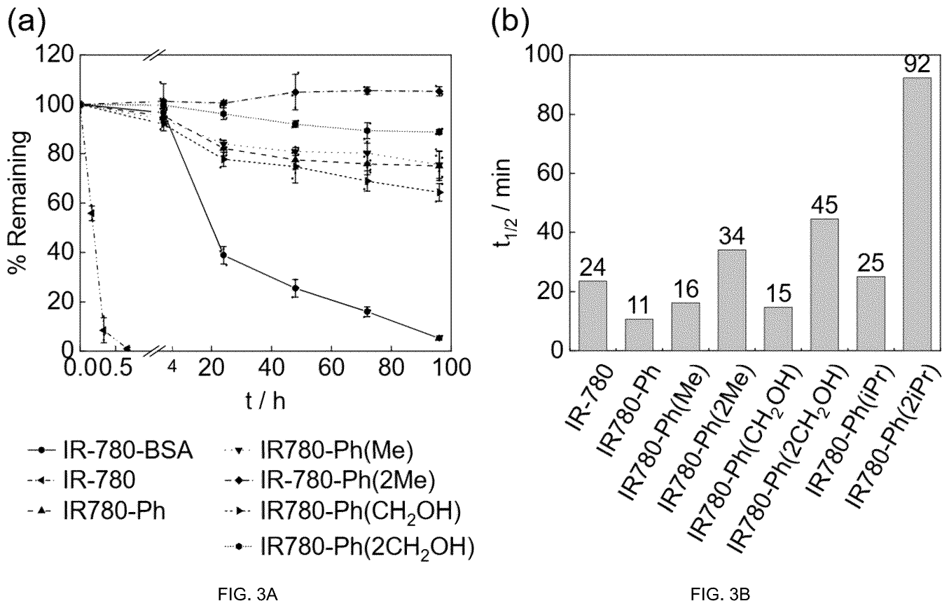

We next sought to verify the stability improvement by the introduction of ortho-substituents on 4′-aryl modifications. Heptamethine dyes, including ICG, undergo degeneration in aqueous condition due to oxidation and/or nucleophilic break down of the methine bridge, and we hypothesize that by introducing steric bulk over the polymethine bridge, such processes can be slowed down to afford more stable fluorophores. We thus incubated the serum solutions of IR-780 and its derivative with 4′-aryl substitution carrying mono-/di-methyl or mono-/di-hydroxymethyl at the ortho-positions at 37° C. in the dark and monitored the remaining dyes in solution over time. As expected, the 4′-position of IR-780 undergoes rapid substitution with nucleophilic side-chains of serum proteins to afford fluorophore-labeled proteins within 40 min (FIG. 3A), which indicates the incompatibility of using 4′-chloroheptamethine fluorophores as a solution in physiological condition. The labeled protein also showed fast degradation as shown by the decrease of absorption, and minimal absorption was left after 4 days (FIG. 3A). In contrast, more than 70% of IR780-Ph remained after 4-day incubation due to the replacement of Cl as a strong leaving group with a robust C—C bonded substitution. The stability is further enhanced with the introduction of a single ortho-methyl group on the 4′-aryl ring, whereas little degradation of IR780-Ph(2Me) with two ortho-methyl groups was observed during the experiment. Although ortho-hydroxymethyl groups made IR780-Ph(CH₂OH) more susceptible to degradation than IR780-Ph, the disubstitution of hydroxymethyl on IR780-Ph(2CH₂OH) resulted in only 10% degradation over four days for this heptamethine-X scaffold (FIG. 3A).

We also characterized the influence of such steric bulk towards the photostability and, similarly, observed enhanced stability with larger functionalities on ortho-positions of the 4′-phenyl substitution. The substitution of 4′-chloro in IR780 with 4′-phenyl in IR780-Ph almost doubles the photobleaching rate, likely due to higher electron density on IR780-Ph that makes the heptamethine bridge more reactive towards photo-generated oxidants (FIG. 3B). On the other hand, the introduction of ortho-substitutions significantly slow down the photobleach rate. Again, while ortho-mono substitution on the 4′-aryl ring modestly increases the photostability, such effect is more pronounced with two ortho-substitutions in heptamethine-X producing fluorophores more photostable than the parent IR780. Notably, a single isopropyl at the ortho-position as in IR780-Ph(iPr) makes the photostability of the fluorophore already comparable to the parent IR780, whereas the introduction of two isopropyl groups in IR780-Ph(2iPr) significantly enhances the photostability, producing a fluorophore with almost four times longer half-life than the parent IR780 under the same illumination condition (FIG. 3B). Collectively, these improvements in stability underscore the efficacy of stability improvement by placing steric protection on both sides of the methine bridge in heptamethine dyes especially with our heptamethine-X scaffold.

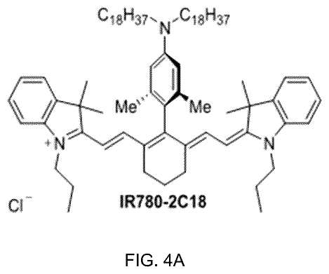

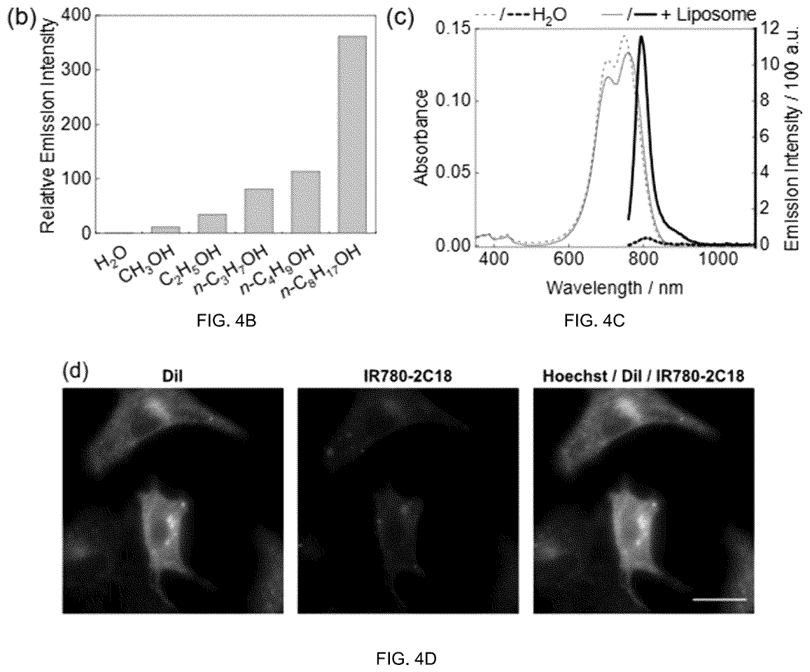

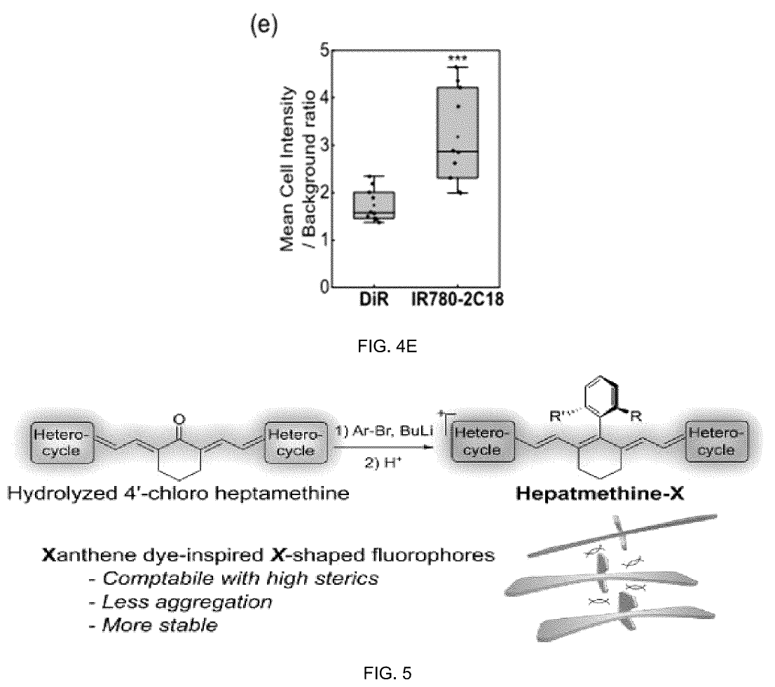

Fluorogenic Cy7 fluorophore for membrane staining: Encouraged by these added benefits with heptamethine-X scaffold, we lastly pursued to realize its utility in creating an improved heptamethine fluorophore for bioimaging. Towards this end, we synthesized fluorophore 4 (IR780-2C18, FIG. 4A) using IR780═O and an aryl bromide from one-step synthesis from commercial compounds. On this fluorophore, the 4′-aryl group carries two ortho-methyl group for stability improvement and aggregation reduction, and two octadecyl groups on the amino functionality at the para-position for membrane localization. Notably, on this molecule, the fluorescence core (from IR780═O) and the targeting group (4′-substitution) are facilely coupled together as a late-stage functionalization, which is an example of convergent synthesis of molecular dyes highlighting the modularity of our method.

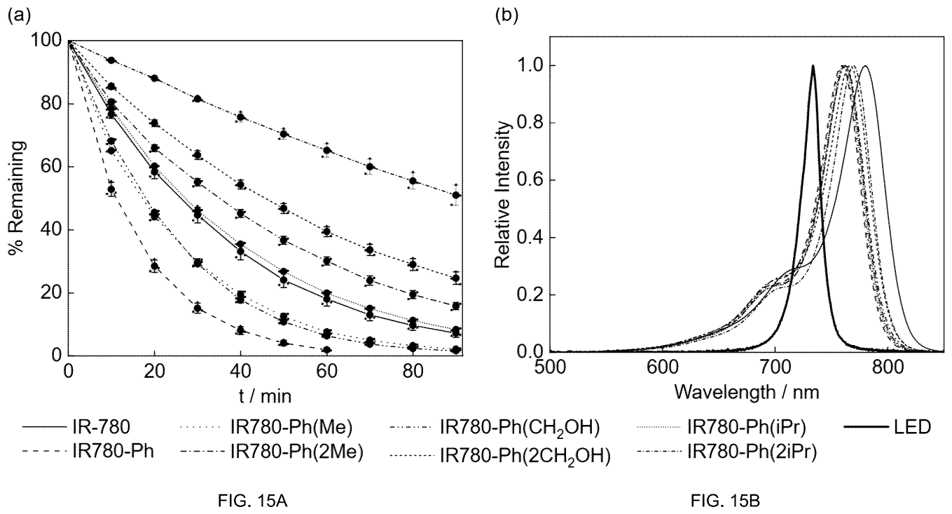

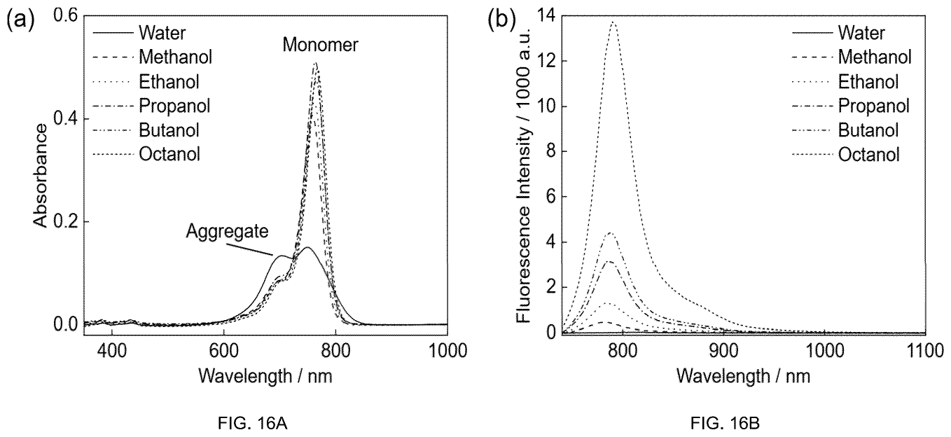

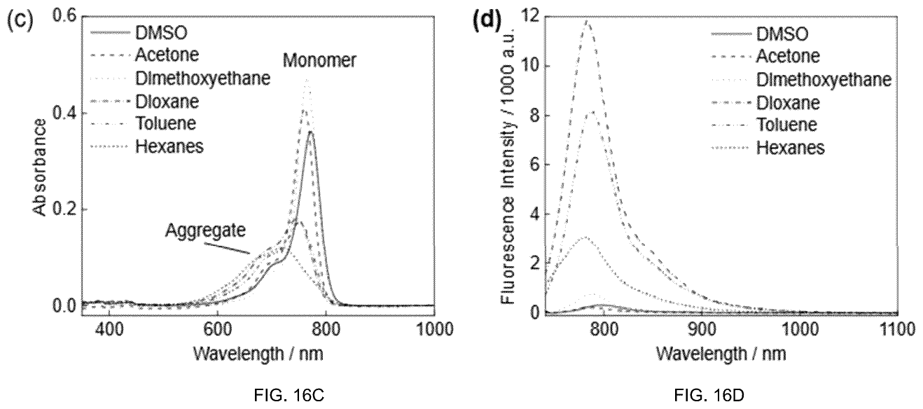

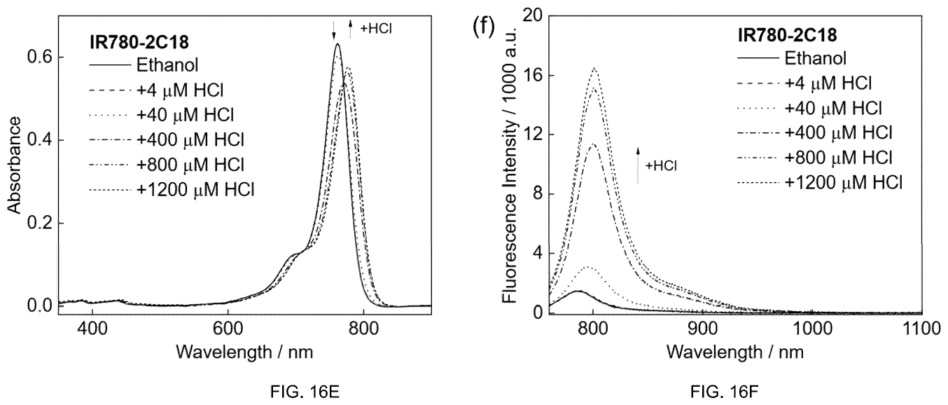

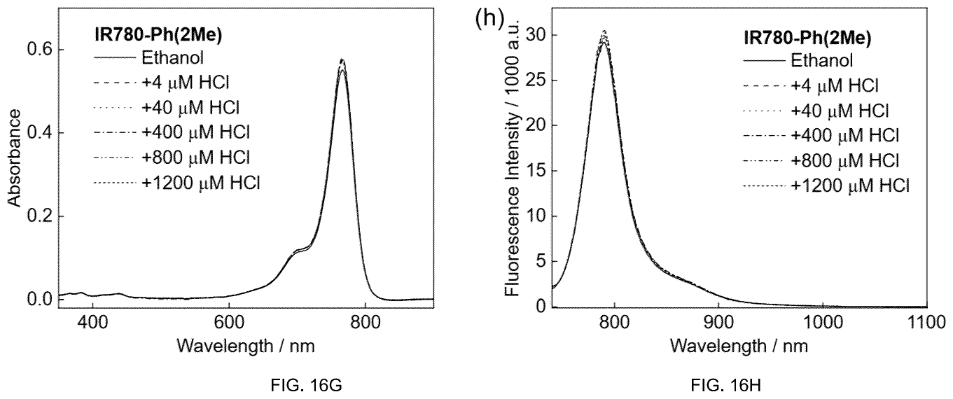

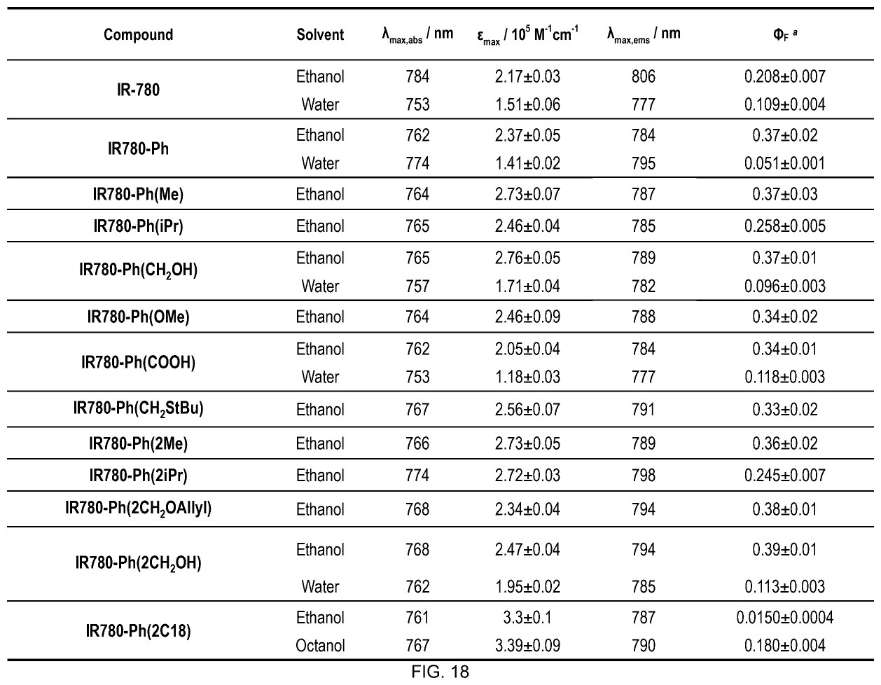

Upon obtaining IR780-2C18, we tested its photophysical behavior. Different from 2a-I, this fluorophore shows minimal fluorescence in ethanol and methanol, whereas the emission intensity increases when longer chain alcohol was used as solvent (FIG. 4B; FIGS. 15A-B). Indeed, while the molecule shows similar absorption coefficients, its fluorescence quantum yield increases from 0.015 in ethanol to 0.18 in octanol (FIG. 18). The 33-fold fluorescence turn-on from methanol to octanol and the 362-fold turn on from water to octanol, a mimic for membrane environment, suggest that IR780-2C18 is a fluorogenic dye for membrane imaging. This phenomenon is further supported by the similar absorption spectra but greatly enhanced emission intensity in the presence of preformed liposomes compared to in water without liposomes (FIG. 4C). Further testing reveals that the emission intensity of IR780-2C18 is higher in more non-polar solvents until aggregation happens (FIGS. 16C-D), suggesting the existence of a charged species during the quenching process. We also observed that in ethanol, the fluorescence of IR780-2C18 can be rescued by adding HCl (FIG. 16E-F), which protonates the amino group on the 4′-aryl substitution while minimally affecting the fluoro-phore core as shown in IR780-Ph (FIGS. 16G-H). These observations combined suggest that this quenching effect of IR780-2C18 in polar conditions likely comes from the photo-induced electron transfer (PeT) from the amino-phenyl ring to the fluorophore, which is less favorable in non-polar solvents due to the charge reorganization. While this quenching process on Cy7 scaffold can be leveraged for designing future biosensors, the fluorogenic effect of IR780-2C18 on membrane mimics make it a promising candidate for wash-free imaging of the cell membrane with higher contrast owing to its fluorogenic property.

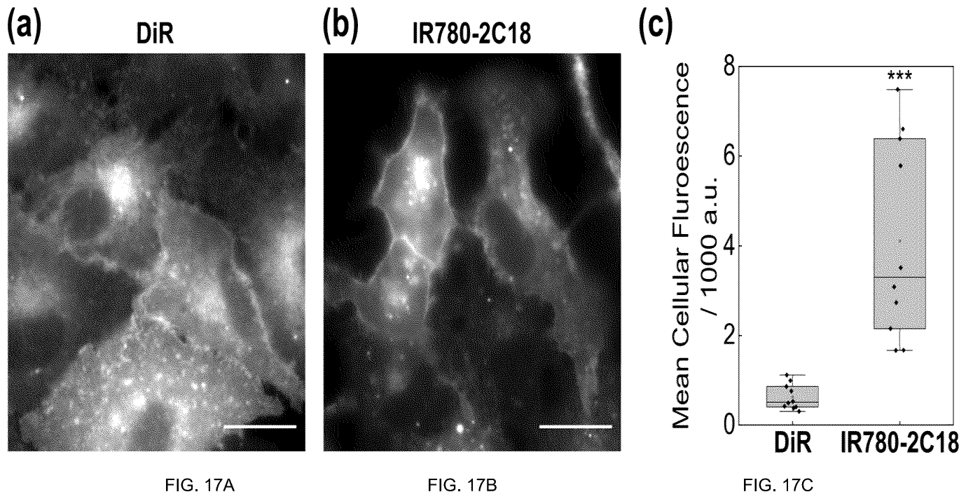

We then carried out epifluorescence microscopy to show the membrane staining property of IR780-2C18. Here, we co-incubated the cells with IR780-2C18 and DiI, a commercial Cy3 derivative bearing two C18 chains on the indolium nitrogen to stain the cell membrane in the yellow channel. Indeed, both dyes outlined the cell contour under microscopy with only slight variation of the bright dots which could be from the aggregation from each dye (FIG. 4D). The high correlation between these two channels (Pearson's R value=0.93 between the two channels) supports the same membrane staining capability of IR780-2C18. We also used DiR (FIG. 10) as a comparison, a commercial Cy7 derivative of DiI. While both dyes successfully stained the cell membrane in wash-free imaging experiment (FIG. 17), the membrane fluorescence to background signal ratio of IR780-2C18 was significantly higher than DiR (FIG. 4E), indicating that the fluorogenic property of IR780-2C18 results in enhanced contrast compared to the commercial dye. Additionally, the cellular brightness of IR780-2C18 was on average 6.6 times higher than that of DiR (FIG. 17C), likely due to the reduced aggregation of this new dye in the complex cell membrane environment owing to the heptamethine-X structure. Taken together, the high contrast and brightness of IR780-2C18 and its retention of the membrane binding property as the commercial dye counterparts highlight the merit of our modular synthesis of functional heptamethine dyes with improved performance.

Concluding Remarks: To close, we have reported a general synthetic strategy to convert common heptamethine fluorophores carrying 4′-Cl substitution into 4′-aryl modifications carrying various substituents linked by robust C—C bonds. The synthesis involves the aryllithium addition to the keto-form of the heptamethine dye, which is analogous to the popular synthesis of xanthene dyes including rhodamine, fluorescein and their derivatives. The transformation is fast, mild and, most importantly, capable of the late-stage modification of existing 4′-chloro heptamethine dyes without the synthesis of the fluorophore core. The high efficiency of this method allows for the introduction of 4′-aryl with high sterics demand exemplified by the heptamethine-X series dyes, which is inaccessible using reported Suzuki coupling pathway. While this strategy is particular useful for furnishing heptamethine dyes with high steric demands, it provides insights in the structural analogy of xanthene dyes and polymethine fluorophores and serve as a further step in consolidating the synthetic methods, design principles and application fields among seemingly different fluorophore families for the next generation of fluorescent probes and sensors.

Besides the new synthetic method, we identified improvements of introducing 4′-aryl substitution on heptamethine dyes especially in heptamethine-X with two ortho-substituents in the center aryl ring. In addition to providing Cy7 derivatives with higher fluorescent quantum yields in organic solvent, introducing C—C bond in place of C—Cl results in dyes more resistant to nucleophlic degradation. Additionally, the increased steric bulk around the methine bridge from ortho-substituents in heptamethine-X can not only boost the stability in the presence of serum proteins or under light, but also remarkably reduce the aggregation propensity in water when two hydroxymethyl groups are incorporated. Through our facile synthesis of fluorogenic membrane staining fluorophore IR780-2C18, we showcased the benefits of our new scaffold with its high brightness and contrast in cell imaging experiments compared with commercial membrane staining dye. We expect the heptamethine-X structure to be employed in the development of future heptamethine dyes, by both our team and others, to enhance stability, decrease aggregation, and introduce hydrophilicity, which is particularly pertinent for challenging fluorophores with extremely red-shifted wavelengths.

PROCEDURES AND CHARACTERIZATION METHODS: Dye handling and storage: All dyes were stored in pure solid form after purification in −20° C. freezer. Stock solutions were prepared as 2 mM solutions in absolute ethanol, and diluted into desired concentration for characterization.

Photophysical characterization: Absorption spectra were collected on a VWR UV-1600PC Scanning Spectrophotometer after blanking with the appropriate solvent. Photoluminescence spectra were obtained on a StellarNet SILVER-Nova spectrometer coupled with a Spectral Products ASTN-W100L-CM light source. Quartz or glass cuvettes (10 mm×10 mm) or polystyrene cuvettes (10 mm×5 mm) were used for absorption measurements. Quartz cuvettes (10 mm×10 mm) were used for photoluminescence measurements. All spectra were obtained at ambient temperature. Fluorescence quantum yield was measured with 730 nm excitation using indocyanine green (ICG) in absolute ethanol as a reference (ϕ_{F}=0.132). Six or more data points were acquired for the calculation of absorption coefficient and quantum yield by linear regression, where the standard error of slopes of the unknowns were used to determine error values.

Stability assay in FBS: Dyes were diluted to 4 μM in 1 mL of bovine calf serum (Hyclone) containing 0.01% w/v NaN3 and placed in disposable cuvettes (10 mm×5 mm). The cuvettes were sealed with Parafilm and placed in 37° C. incubator until given time points. Absorption spectra were taken using the maximum absorption to represent the dye concentration. For cysteine-reacted IR-780, the pristine IR-780 was pre-incubated in serum at 37° C. for 1 h for the reaction to complete before starting the experiment. For IR-780, the incubation was carried out as 4 μM, 250 μL solutions in microcentrifuge tube, quenched until given time points with 800 μL cold methanol, centrifuged (1,7000×g, 5 min) to remove precipitated serum proteins, and measured on spectrometer. Each condition was performed in triplicate.

Photostability assay: Dyes were diluted to 4 μM in 1 mL of 1:1 methanol/water and placed in disposable cuvettes (10 mm×5 mm). The cuvettes were shined through the 5 mm light path in front of a self-made LED light (LEDLightsWorld, 730 nm LED strips) matrix (6.8 mW/cm₂) until given time points. Absorption spectra were taken using the maximum absorption to represent the dye concentration.

SYNTHESIS: Synthetic materials and methods: Unless otherwise noted, all commercial reagents were used without further purification. All reactions utilizing air-or moisture-sensitive reagents were performed under an atmosphere of dry N₂. Dry solvents were purchased from Thermo Scientific Chemicals and stored over sieves under an atmosphere of dry N₂. Chemical reagents were purchased from Ambeed, Oakwood Chemicals and Thermo Scientific Chemicals. Heptamethine dyes were purchased from Thermo Scientific Chemicals (IR-780, IR-775), TCI America (IR-813) and Enamine (IR-1061).

Chrom7, (2-bromobenzyl)(tert-butyl)sulfane, and 2-bromo-1,3-benzenedimethanol was synthesized according to published procedures. 1H NMR and 13C NMR spectra were collected in CDCl3, CD3CN or MeOD at 25° C. on Bruker 400 MHz or 500 MHz spectrometers at the NMR Facility at the Department of Chemistry and Biochemistry in the University of Arkansas. All chemical shifts in 1H NMR and 13C NMR are reported in the standard notation of ppm relative to residual solvent peak (CDCl3 δH=7.26, δC=77.16; CD3CN δH=1.94, δC=1.32; MeOD δH=3.31, δC=49.00; CD2Cl2 δH=5.32, δC=53.84). High resolution mass spectrometry was acquired on an IT-TOF (Shimadzu) at the University of Arkansas Statewide Mass Spectrometry Facility.

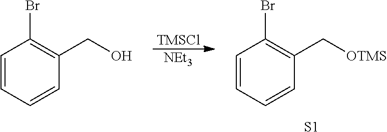

((2-Bromo-3-methylbenzyl)oxy)trimethylsilane (S1): To a flask containing 2-Bromophenylmethanol (200 mg, 1.1 mmol) and triethylamine (0.22 mL, 1.6 mmol) dissolved in CH2Cl2 (10 mL) was added trimethylsilyl chloride (0.16 mL, 1.3 mmol). The mixture was stirred for 2.5 h at room temperature and concentrated to dryness. The crude product was separated by column chromatography (1:50 ethyl acetate/hexanes) to give S1 as a colorless liquid (274 mg, 99%), which is used without further purification. ¹H NMR (400 MHz, CDCl3) δ 7.53 (d, J=7.7 Hz, 1H), 7.50 (d, J=8.1 Hz, 1H), 7.32 (t, J=7.5 Hz, 1H), 7.12 (t, J=7.5 Hz, 1H), 4.72 (s, 2H), 0.19 (s, 9H). This compound has also been characterized elsewhere.

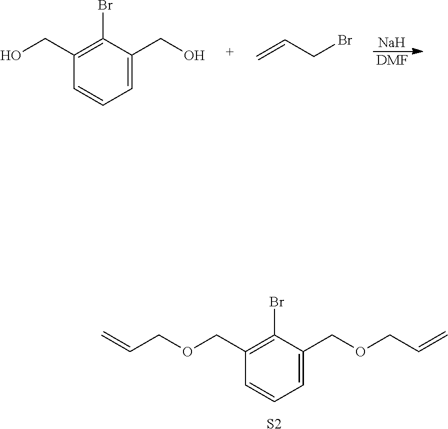

1,3-Bis((allyloxy)methyl)-2-bromobenzene (S2): To a flask containing 2-bromo-1,3-benzenedimethanol (305 mg, 1.41 mmol) and NaH (60% dispersion in mineral oil, 281 mg, 7.03 mmol) under N2 was added dry DMF (7 mL). The reaction was stirred at room temperature for 0.5 h, followed by the dropwise addition of allyl bromide (0.35 mL, 4.2 mmol). The reaction was further stirred or 1 h, quenched with addition of MeOH and H₂O, and extracted into ethyl acetate. The organic layer was washed with H2O (×4) and saturated NaCl, dried (Na₂SO₄) and concentrated. The crude product was separated by column chromatography (1:50 ethyl acetate/hexanes) to give S2 as a colorless liquid (417 mg, >99%). ¹H NMR (400 MHz, CDCl3) δ 7.44 (d, J=7.5 Hz, 2H), 7.33 (t, J=7.7 Hz, 1H), 5.99 (ddd, J=22.8, 10.8, 5.6 Hz, 2H), 5.36 (dq, J=17.2, 1.7 Hz, 2H), 5.24 (dd, J=10.4, 1.6 Hz, 2H), 4.61 (s, 4H), 4.12 (dt, J=5.6, 1.5 Hz, 4H). ¹³C NMR (101 MHz, CDCl3) δ 138.16, 134.67, 127.98, 127.32, 122.84, 117.36, 71.84, 71.79.

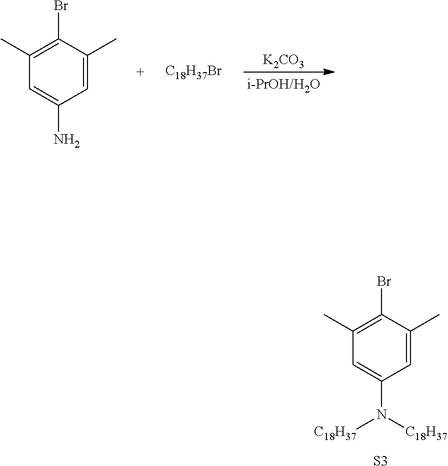

4-Bromo-3,5-dimethyl-N,N-dioctadecylaniline (S3): To a microwave vessel containing 4-bromo-3,5-dimethylaniline (0.20 g, 1.0 mmol), 1-bromooctadecane (1.67 g, 5.0 mmol) and K₂CO₃ (0.55 g, 4.0 mmol) was added 15 mL of 1:2 H₂O/isopropanol. The reaction was carried out in a CEM Discover SP Microwave reactor at 120° C. for 4 h. The mixture was cooled, diluted with H₂O and extracted with CH2Cl2 (×5), dried (Na₂SO₄) and concentrated. The crude product was separated by column chromatography (hexanes) to give S3 as a white solid (481 mg, 68%). ¹H NMR (500 MHz, CDCl₃) δ 6.38 (s, 1H), 3.20 (t, J=7.6 Hz, 2H), 2.36 (s, 3H), 1.60-1.51 (m, 2H), 1.36-1.22 (m, 34H), 0.90 (t, J=6.9 Hz, 3H). ¹H NMR (500 MHz, CDCl3) δ 6.38 (s, 2H), 3.20 (t, J=7.6 Hz, 4H), 2.36 (s, 6H), 1.60-1.51 (m, 4H), 1.36-1.22 (m, 64H), 0.90 (t, J=6.9 Hz, 6H). ¹³C NMR (126 MHz, CDCl3) δ 147.12, 138.48, 113.07, 112.27, 51.23, 32.11, 29.88, 29.80, 29.67, 29.53, 27.38, 27.34, 24.49, 22.85, 14.24. HRMS (ESI+) calcd 704.5703, found 704.5695 for C₄₄H₈₃BrN+ (M+H+).

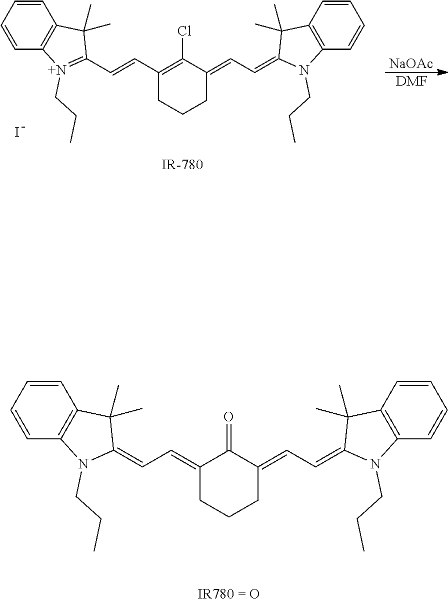

2,6-Bis(2-(3,3-dimethyl-1-propylindolin-2-ylidene)ethylidene)cyclohexan-1-one (IR780═O): To a flask containing IR-780 iodide (500 mg, 0.75 mmol) and sodium acetate (184 mg, 2.25 mmol) was added dry DMF (10 mL) followed by three freeze-pump-thaw cycles. The reaction was then stirred at 80° C. under N₂ for 3 h. The mixture was diluted in ethyl acetate, washed with H₂O (×4) and saturated NaCl, dried (Na₂SO₄) and concentrated. The crude product was separated by column chromatography (1:7.5 ethyl acetate/hexanes) to give IR-780═O as a dark red solid (365 mg, 93%). ¹H NMR (400 MHz, CDCl3) δ 8.17 (d, J=13.2 Hz, 2H), 7.26-7.13 (m, 4H), 6.90 (t, J=7.4 Hz, 2H), 6.68 (d, J=8.0 Hz, 2H), 5.46 (d, J=13.2 Hz, 2H), 3.64 (t, J=7.4 Hz, 4H), 2.61 (t, J=6.2 Hz, 4H), 1.91-1.83 (m, 2H), 1.76 (h, J=7.5 Hz, 4H), 1.67 (s, 12H), 1.01 (t, J=7.4 Hz, 6H). ¹³C NMR (101 MHz, CDCl3) δ 186.45, 162.51, 144.48, 139.77, 132.97, 127.68, 126.58, 121.84, 120.48, 106.81, 92.63, 46.63, 44.21, 28.88, 25.93, 22.68, 19.84, 11.84. HRMS (ESI+) calcd 521.3526, found 521.3530 for C₃₆H₄₅N₂O+ (M+H+).

General procedure A: preparation of heptamethine-O. The preparation is adapted from previous reports. Specifically, heptamethine-Cl (1.0 equiv.), N-hydroxysuccinimide (NHS, 3.0 equiv.) and N,N-diisopropylethylamine (3.0 equiv.) were dissolved in DMF and stirred at room temperature until complete conversion of the starting heptamethine-Cl as determined by TLC. The mixture was then diluted in ethyl acetate, washed with H₂O (×4) and saturated NaCl, dried (Na₂SO₄) and concentrated. The crude product was separated by column chromatography (1:200 methanol/CH₂Cl₂) to give heptamethine-O.

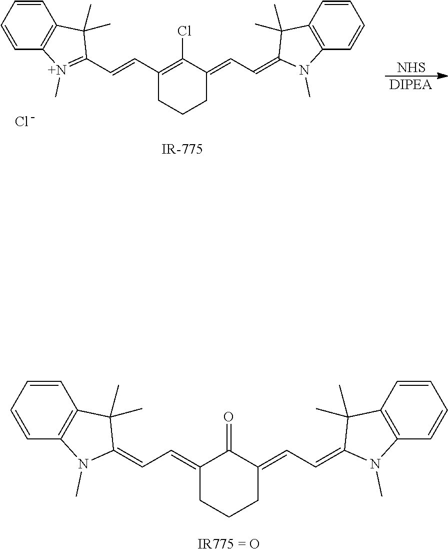

2,6-Bis(2-(1,3,3-trimethylindolin-2-ylidene)ethylidene)cyclohexan-1-one (IR775═O): Following General Procedure A, IR-775 chloride (50 mg, 0.096 mmol) was reacted with NHS (33 mg, 0.29 mmol) and DIPEA (50 μL, 0.29 mmol) in dry DMF (2 mL) for 18 h to give IR775═O as a dark red-orange solid (31 mg, 70%). ¹H NMR (400 MHz, CDCl3) δ 8.18 (d, J=13.2 Hz, 2H), 7.18 (t, J=7.3 Hz, 4H), 6.90 (t, J=7.4 Hz, 2H), 6.68 (d, J=7.9 Hz, 2H), 5.41 (d, J=13.2 Hz, 2H), 3.21 (s, 6H), 2.62 (t, J=6.2 Hz, 4H), 1.87 (p, J=6.4 Hz, 2H), 1.68 (s, 12H). ¹³C NMR (101 MHz, CDCl3) δ 186.60, 163.33, 144.68, 139.70, 132.91, 127.73, 126.93, 121.80, 120.59, 106.52, 92.65, 46.52, 29.36, 28.78, 25.95, 22.63. HRMS (ESI+) calcd 465.2900, found 465.2928 for C₃₂H₃₇N₂O+ (M+H+).

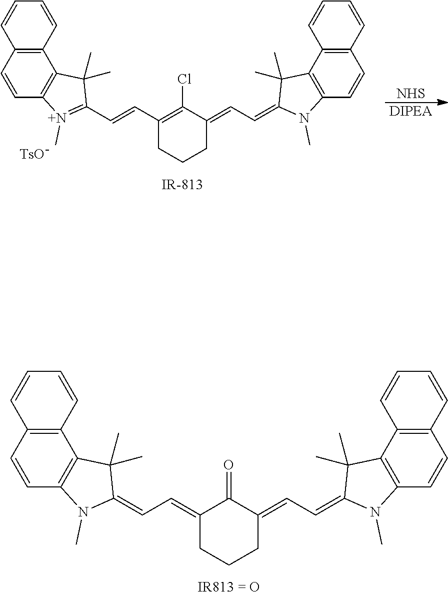

2,6-Bis(2-(1, 1,3-trimethyl-1,3-dihydro-2H-benzo[e]indol-2 ylidene)ethylidene)cyclohexan-1-one (IR813═O): Following General Procedure A, IR-813 p-toluenesulfonate (100 mg, 0.152 mmol) was reacted with NHS (46 mg, 0.40 mmol) and DIPEA (69 μL, 0.40 mmol) for 1 h to give IR813═O as a deep red to dark magenta solid (39.5 mg, 54%). ¹H NMR (400 MHz, CDCl3) δ 8.37 (d, J=13.2 Hz, 2H), 8.07 (d, J=8.6 Hz, 2H), 7.83 (d, J=8.3 Hz, 2H), 7.77 (d, J=8.8 Hz, 2H), 7.50 (ddd, J=8.3, 6.7, 1.3 Hz, 2H), 7.33-7.25 (m, 2H), 7.10 (d, J=8.7 Hz, 2H), 5.48 (d, J=13.3 Hz, 2H), 3.33 (s, 6H), 2.69 (t, J=5.5 Hz, 4H), 2.04 (s, 12H), 1.97-1.90 (m, 2H). ¹³C NMR (101 MHz, CDCl3) δ 186.53, 165.26, 141.92, 132.81, 130.04, 129.85, 129.82, 129.48, 129.07, 126.93, 126.81, 122.60, 121.93, 109.10, 92.37, 48.54, 29.61, 28.05, 25.99, 22.69. HRMS (ESI+) calcd 565.3213, found 565.3235 for C₄₀H₄₁N₂O+ (M+H+).

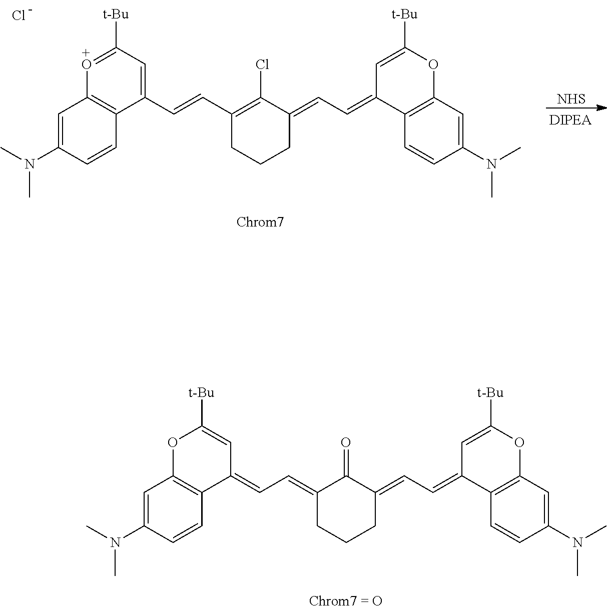

2,6-Bis(2-(2-(tert-butyl)-7-(dimethylamino)-4H-chromen-4-ylidene)ethylidene)cyclohexan-1-one (Chrom7═O): Following General Procedure A, Chrom7 chloride (40 mg, 0.061 mmol) was reacted with NHS (23 mg, 0.20 mmol) and DIPEA (32 μL, 0.18 mmol) for 2 h to give Chrom7═O as a dark purple solid (10 mg, 28%). ¹H NMR (400 MHz, CDCl3) δ 8.03 (d, J=12.8 Hz, 2H), 7.63 (d, J=9.0 Hz, 2H), 6.58 (dd, J=9.0, 2.6 Hz, 2H), 6.49 (s, 2H), 6.39-6.28 (m, 4H), 3.01 (s, 12H), 2.74 (t, J=5.9 Hz, 4H), 1.87 (p, J=5.9 Hz, 2H), 1.28 (s, 18H). ¹³C NMR (101 MHz, CDCl3) δ 163.37, 154.01, 152.03, 135.45, 131.76, 130.44, 123.79, 111.21, 109.88, 104.82, 98.94, 97.89, 40.36, 35.86, 28.18, 26.66, 22.69. HRMS (ESI+) calcd 605.3738, found 605.3730 for C₄₀H₄₉N₂O₃ (M+H+).

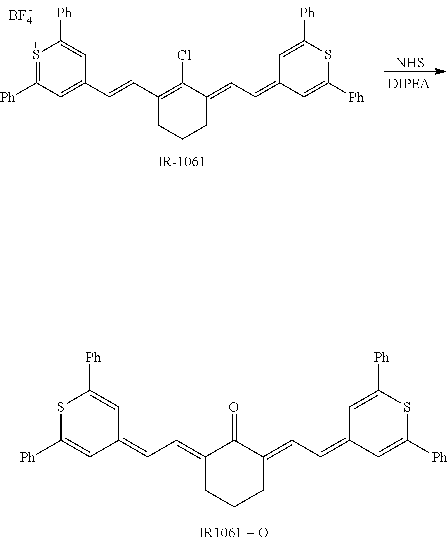

2,6-Bis(2-(2,6-diphenyl-4H-thiopyran-4-ylidene)ethylidene)cyclohexan-1-one (IR1061═O): Following General Procedure A, IR-1061 tetrafluoroborate (95 mg, 0.13 mmol) was reacted with NHS (48 mg, 0.42 mmol) and DIPEA (66 μL, 0.38 mmol) for 2 h to give IR1061═O as a dark brown solid (50 mg, 61%). ¹H NMR (400 MHz, CDCl3) δ 8.00 (d, J=12.8 Hz, 2H), 7.69-7.53 (m, 8H), 7.50-7.36 (m, 14H), 6.86 (s, 2H), 6.08 (d, J=13.0 Hz, 2H), 2.68 (t, J=6.0 Hz, 4H), 1.85 (p, J=6.0 Hz, 2H). ¹³C NMR (101 MHz, CDCl3) δ 187.77, 140.00, 139.87, 137.99, 137.64, 137.58, 132.49, 130.76, 129.66, 129.43, 129.06, 126.43, 126.37, 126.13, 119.57, 118.58, 26.58, 22.30. HRMS (ESI+) calcd 643.2124, found 643.2086 for C₄₄H₃₅OS₂+ (M+H+).

General procedure B: preparation of meso-substituted heptamethine dyes. Unless otherwise noted, aryl bromide (0.51 mmol) was dissolved in dry THF (1 mL) and cooled to −84° C. To this solution was added n-BuLi (2.3 M in cyclohexane/hexanes, 147 μL, 0.34 mmol). The mixture was stirred for 10 min at this temperature. A red to orange solution of keto-heptamethine (0.042 mmol) in dry THF (1 mL) was added dropwise. The mixture was allowed to warm up to room temperature and stirred for 30 min. The yellow or orange reaction mixture was quenched by adding 1:10 HCl which resulted in a rapid color change into dark green. The mixture was diluted in H2O and extracted with CH₂Cl₂ (×4) and dried (Na₂SO₄). The crude product was purified by column chromatography (1:30 to 1:20 methanol/CH₂Cl₂).

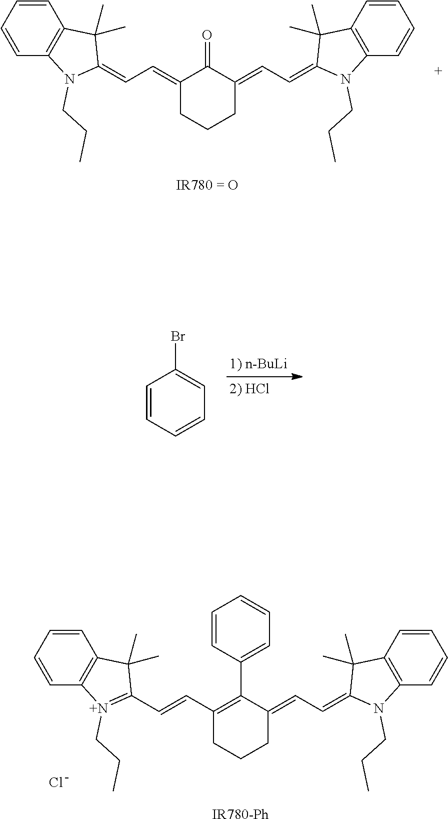

2-(2-(6-(2-(3,3-Dimethyl-1-propylindolin-2-ylidene)ethylidene)-3,4,5,6-tetrahydro-[1,1′-biphenyl]-2-yl)vinyl)-3,3-dimethyl-1-propyl- 3H-indol-1-ium chloride (IR780-Ph): Following General Procedure B, IR780═O (22 mg, 0.042 mmol) was reacted with bromobenzene (80 mg, 0.51 mmol) and n-BuLi (2.3 M, 0.15 mL, 0.34 mmol) to give IR780-Ph as a green solid (27 mg, >99%) ¹H NMR (400 MHz, MeOD with a few drops of CDCl3) δ 7.65-7.52 (m, 3H), 7.33 (td, J=7.6, 1.2 Hz, 2H), 7.29-7.21 (m, 6H), 7.16 (t, J=7.0 Hz, 4H), 6.11 (d, J=14.0 Hz, 2H), 4.01 (t, J=7.3 Hz, 4H), 2.70 (t, J=6.3 Hz, 4H), 2.07 (p, J=6.3 Hz, 2H), 1.84 (h, J=7.4 Hz, 4H), 1.17 (s, 12H), 1.02 (t, J=7.4 Hz, 6H). ¹³C NMR (101 MHz, MeOD with a few drops of CDCl3) δ 172.91, 163.77, 149.34, 143.28, 141.61, 139.86, 132.19, 130.32, 129.50, 129.41, 129.13, 125.68, 122.94, 111.40, 100.49, 49.53, 46.22, 28.03, 25.43, 22.10, 21.45, 11.76. HRMS (ESI+) calcd 581.3890, found 581.3887 for C₄₂H₄₉N₂+ (M+).

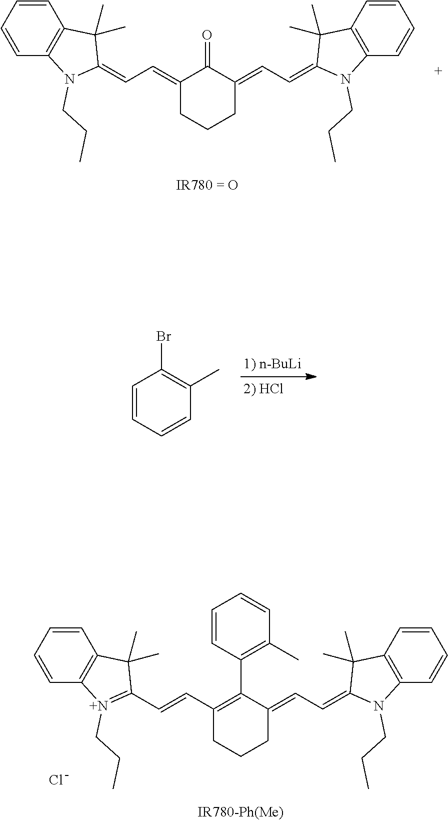

2-(2-(6-(2-(3,3-Dimethyl-1-propylindolin-2-ylidene)ethylidene)-2′-methyl-3,4,5,6-tetrahydro-[1,1′-biphenyl]-2-yl)vinyl)-3,3- dimethyl-1-propyl-3H-indol-1-ium chloride [IR780-Ph(Me)]: Following General Procedure B, IR780═O (22 mg, 0.042 mmol) was reacted with 2-bromotoluene (87 mg, 0.51 mol) and n-BuLi (2.3 M, 0.15 mL, 0.34 mmol) to give IR780-Ph(Me) as a green solid film (23 mg, 93%). ¹H NMR (400 MHz, MeOD) δ 7.51-7.40 (m, 3H), 7.39-7.28 (m, 4H), 7.25-7.15 (m, 6H), 7.09 (d, J=7.3 Hz, 1H), 6.19 (d, J=14.0 Hz, 2H), 4.05 (t, J=7.3 Hz, 4H), 2.73 (t, J=6.3 Hz, 4H), 2.14 (s, 2H), 2.07 (p, J=6.2 Hz, 2H), 1.83 (h, J=7.4 Hz, 4H), 1.16 (s, 6H), 1.13 (s, 6H), 1.01 (t, J=7.4 Hz, 6H). ¹³C NMR (101 MHz, MeOD) δ 172.05, 161.89, 147.06, 142.37, 140.75, 138.23, 135.93, 130.43, 130.33, 129.15, 128.37, 126.11, 124.61, 121.97, 115.00, 110.49, 99.57, 53.48, 48.52, 45.00, 26.86, 26.66, 24.12, 21.22, 20.35, 10.33. HRMS (ESI+) calcd 595.4048, found 595.4047 for C₄₃H₅₁N₂+ (M+).

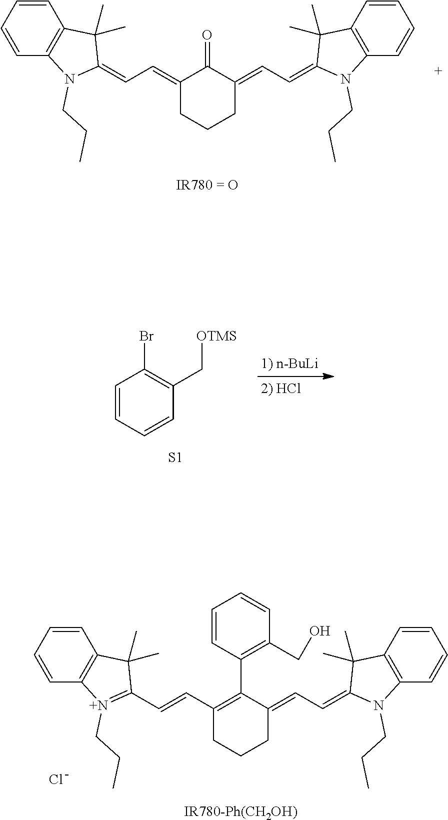

2-(2-(6-(2-(3,3-Dimethyl-1-propylindolin-2-ylidene)ethylidene)-2′-(hydroxymethyl)-3,4,5,6-tetrahydro-[1,1′-biphenyl]-2- yl)vinyl)-3,3-dimethyl-1-propyl-3H-indol-1-ium chloride [IR780-Ph(CH2OH)]: Following General Procedure B, IR780═O (22 mg, 0.042 mmol) was reacted with ((2-bromo-3-methylbenzyl)oxy)trimethylsilane (131 mg, 0.51 mol) and n-BuLi (2.3 M, 0.15 mL, 0.34 mmol) to give IR780-Ph(CH2OH) as a green solid film (21 mg, 78%). ¹H NMR (400 MHz, MeOD) δ 7.80 (d, J=7.7 Hz, 1H), 7.61 (t, J=7.6 Hz, 1H), 7.51 (t, J=7.4 Hz, 1H), 7.38-7.29 (m, 4H), 7.27-7.14 (m, 6H), 7.11 (d, J=7.4 Hz, 1H), 6.19 (d, J=14.1 Hz, 2H), 4.45 (s, 2H), 4.05 (t, J=7.4 Hz, 4H), 2.73 (s, 4H), 2.07 (p, J=6.5 Hz, 2H), 1.82 (h, J=7.5 Hz, 4H), 1.17 (s, 6H), 1.14 (s, 6H), 1.01 (t, J=7.4 Hz, 6H). ¹³C NMR (101 MHz, MeOD) δ 173.52, 148.67, 143.74, 142.21, 141.13, 137.89, 132.04, 130.54, 129.76, 129.67, 128.60, 128.49, 125.94, 123.30, 111.83, 101.01, 62.00, 49.93, 46.34, 28.27, 28.02, 25.52, 22.59, 21.72, 11.65. HRMS (ESI+) calcd 611.3996, found 611.4005 for C₄₃H₅₁N₂O+ (M+).

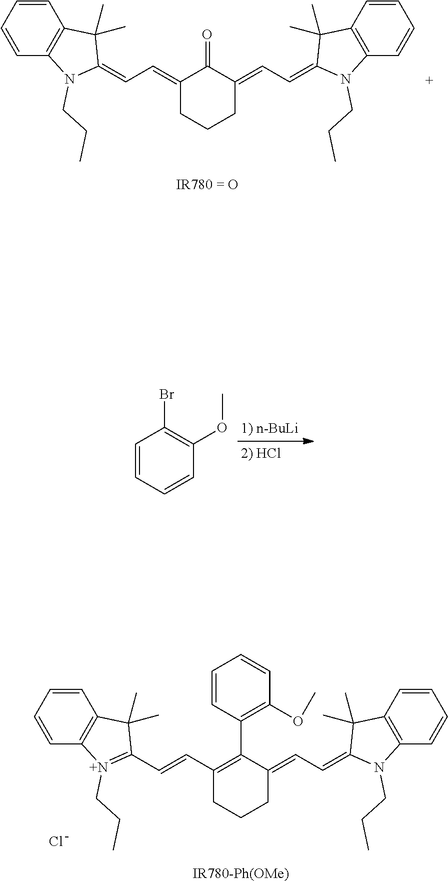

2-(2-(6-(2-(3,3-Dimethyl-1-propylindolin-2-ylidene)ethylidene)-2′-methoxy-3,4,5,6-tetrahydro-[1,1′-biphenyl]-2-yl)vinyl)-3,3-dimethyl-1-propyl-3H-indol-1-ium chloride [IR780-Ph(OMe)]: Following General Procedure B, IR780═O (22 mg, 0.042 mmol) was reacted with 2-bromoanisole (95 mg, 0.51 mol) and n-BuLi (2.3 M, 0.15 mL, 0.34 mmol) to give IR780-Ph(OMe) as a green solid film (25 mg, 98%). ¹H NMR (400 MHz, MeOD) δ 7.59 (t, J=8.0 Hz, 1H), 7.38-7.26 (m, 7H), 7.25-7.13 (m, 5H), 7.08 (dd, J=7.4, 1.7 Hz, 1H), 6.16 (d, J=14.1 Hz, 2H), 4.04 (t, J=7.4 Hz, 4H), 3.75 (s, 3H), 2.70 (s, 4H), 2.14-2.06 (m, 2H), 1.82 (h, J=7.5 Hz, 4H), 1.21 (s, 6H), 1.16 (s, 6H), 1.02 (t, J=7.4 Hz, 6H). ¹³C NMR (101 MHz, MeOD) δ 172.92, 157.89, 148.61, 143.81, 142.08, 131.93, 131.32, 129.67, 128.43, 125.80, 123.30, 122.10, 112.60, 111.72, 100.81, 56.27, 49.81, 46.28, 28.16, 27.96, 25.56, 22.47, 21.69, 11.67. HRMS (ESI+) calcd 611.3996, found 611.4004 for C₄₃H₅₁N₂O+ (M+).

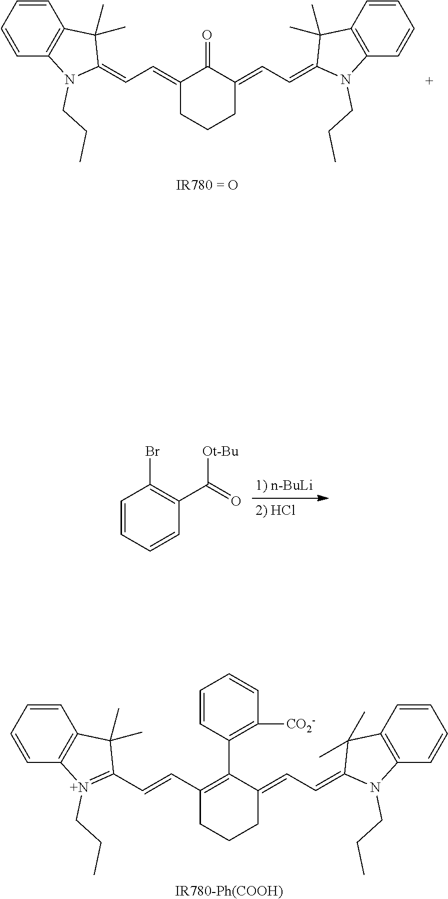

2-(2-(2′-Carboxy-6-(2-(3,3-dimethyl-1-propylindolin-2-ylidene)ethylidene)-3,4,5,6-tetrahydro-[1,1′-biphenyl]-2-yl)vinyl)-3,3-dimethyl-1-propyl-3H-indol-1-ium chloride [IR780-Ph(COOH)]: Following General Procedure B, IR780═O (22 mg, 0.042 mmol) was reacted with tert-butyl 2-bromobenzoate (130 mg, 0.51 mol) and n-BuLi (2.3 M, 0.15 mL, 0.34 mmol) to give IR780-Ph(COOH) as a green solid (13 mg, 50%). ¹H NMR (400 MHz, MeOD) δ 8.20 (d, J=7.7 Hz, 1H), 7.70 (dt, J=27.0, 7.6 Hz, 2H), 7.37-7.29 (m, 4H), 7.26-7.11 (m, 7H), 6.15 (d, J=14.0 Hz, 2H), 4.02 (t, J=7.3 Hz, 4H), 2.70 (t, J=6.2 Hz, 4H), 2.17-1.96 (m, 2H), 1.81 (h, J=7.3 Hz, 4H), 1.19 (s, 6H), 1.12 (s, 6H), 1.00 (t, J=7.4 Hz, 6H). ¹³C NMR (101 MHz, MeOD) δ 172.97, 164.94, 148.86, 143.83, 142.12, 141.00, 132.65, 132.58, 132.15, 131.90, 129.63, 125.73, 123.28, 111.65, 100.71, 49.80, 46.24, 28.30, 27.97, 25.78, 22.30, 21.66, 11.64. HRMS (ESI+) calcd 625.3789, found 625.3784 for C₄₃H₄₉N₂O₂+ (M+H+).

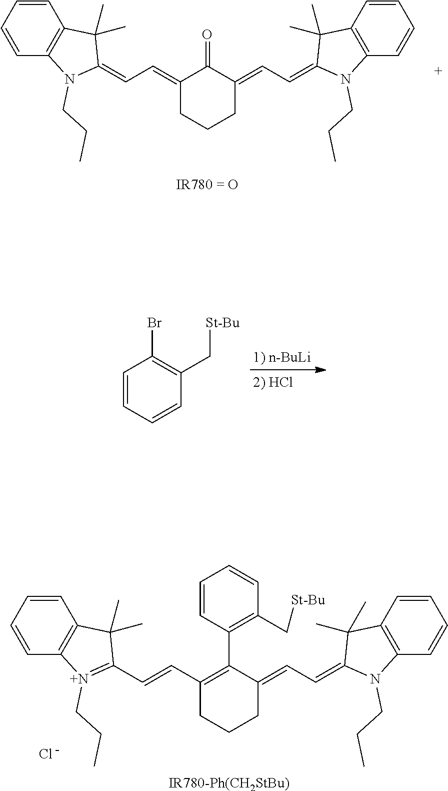

2-(2-(2′-((tert-Butylthio)methyl)-6-(2-(3,3-dimethyl-1-propylindolin-2-ylidene)ethylidene)-3,4,5,6-tetrahydro-[1,1′-biphenyl]-2- yl)vinyl)-3,3-dimethyl-1-propyl-3H-indol-1-ium chloride [IR780-Ph(CH2StBu)]: Following General Procedure B, IR780═O (22 mg, 0.042 mmol) was reacted with (2-bromobenzyl)(tert-butyl)sulfane (133 mg, 0.51 mol) and n-BuLi (2.3 M, 0.15 mL, 0.34 mmol) to give IR780-Ph(CH2StBu) as a green solid film (29 mg, 95%). ¹H NMR (400 MHz, MeOD) δ 7.62 (dd, J=7.6, 1.5 Hz, 1H), 7.51 (dtd, J=19.7, 7.4, 1.5 Hz, 2H), 7.39-7.30 (m, 4H), 7.29-7.10 (m, 7H), 6.20 (d, J=14.1 Hz, 2H), 4.07 (t, J=7.3 Hz, 4H), 3.63 (s, 2H), 2.85-2.64 (m, 4H), 2.18-2.00 (m, 2H), 1.83 (h, J=7.4 Hz, 4H), 1.28-1.21 (m, 15H), 1.17 (s, 6H), 1.01 (t, J=7.4 Hz, 6H). ¹³C NMR (101 MHz, MeOD) δ 173.51, 161.64, 149.13, 143.75, 142.19, 139.52, 138.01, 132.16, 131.83, 131.20, 129.90, 129.66, 128.44, 125.94, 123.30, 111.83, 100.97, 49.98, 46.33, 43.71, 31.16, 30.94, 28.50, 28.05, 25.69, 22.62, 21.74, 11.67. HRMS (ESI+) calcd 683.4393, found 683.4369 for C₄₇H₅₉N₂S+ (M+).

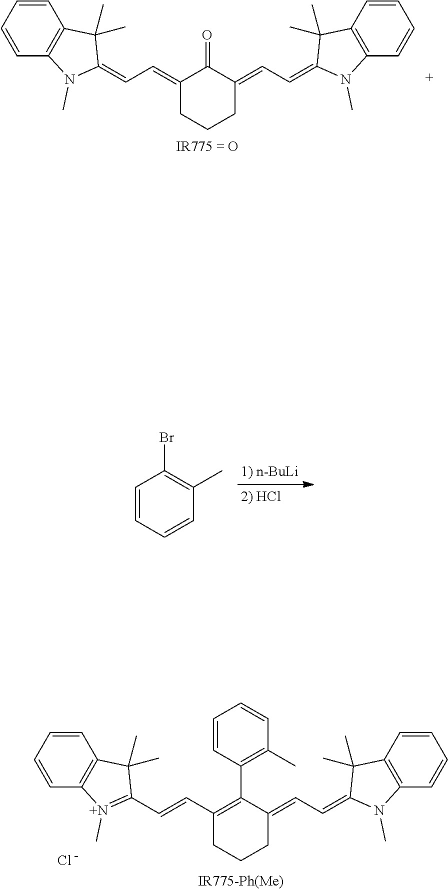

1,3,3-Trimethyl-2-(2-(2′-methyl-6-(2-(1,3,3-trimethylindolin-2-ylidene)ethylidene)-3,4,5,6-tetrahydro-[1,1′-biphenyl]-2- yl)vinyl)-3H-indol-1-ium chloride [(IR775-Ph(Me)]: Following General Procedure B, IR775═O (18 mg, 0.038 mmol) was reacted with 2 bromotulene (78 mg, 0.46 mmol) and n-BuLi (2.3 M, 0.13 mL, 0.31 mmol) to give IR775-Ph(Me) as a green solid (17 mg, 80%). ¹H NMR (400 MHz, CDCl3) δ 7.47-7.27 (m, 5H), 7.19-7.02 (m, 9H), 6.07 (d, J=14.0 Hz, 2H), 3.60 (s, 6H), 2.69 (s, 4H), 2.08 (s, 3H), 2.08-1.98 (m, 2H), 1.10 (d, J=4.7 Hz, 12H). ¹³C NMR (101 MHz, CDCl3) δ 172.11, 162.35, 147.19, 142.87, 140.65, 138.06, 136.02, 131.46, 130.41, 129.31, 128.74, 128.58, 126.23, 124.88, 121.97, 110.49, 100.26, 48.57, 31.80, 27.74, 27.57, 24.72, 21.32, 18.90. HRMS (ESI+) calcd 539.3421, found 539.3418 for C₃₉H₄₃N₂+ (M+).

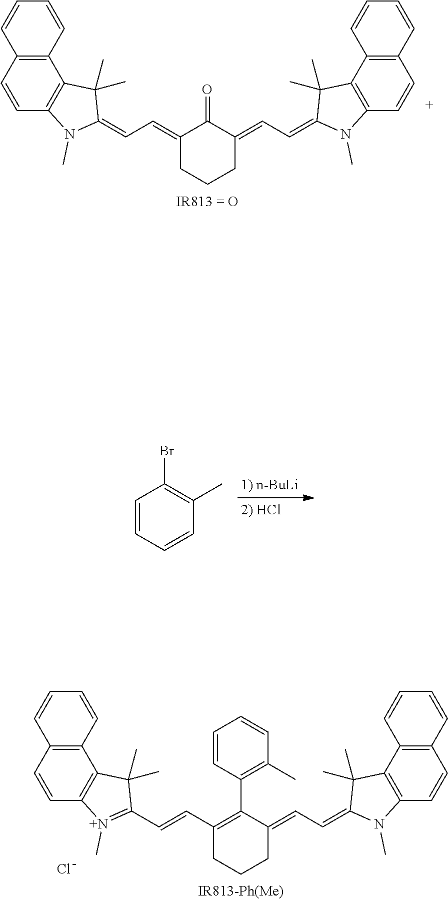

1,1,3-Trimethyl-2-(2-(2′-methyl-6-(2-(1,1,3-trimethyl-1,3-dihydro-2H-benzo[e]indol-2-ylidene)ethylidene)-3,4, 5,6-tetrahydro-[1,1′-biphenyl]-2-yl)vinyl)-1H-benzo[e]indol-3-ium chloride [IR775-Ph(Me)]: Following General Procedure B, IR813═O (25 mg, 0.045 mmol) was reacted with 2-bromotulene (92 mg, 0.54 mmol) and n-BuLi (2.3 M, 0.16 mL, 0.36 mmol) to give IR813-Ph(Me) as a yellowish-green solid (20 mg, 67%). ¹H NMR (400 MHz, CDCl3) δ 8.00-7.83 (m, 6H), 7.62-7.36 (m, 9H), 7.20 (d, J=14.1 Hz, 2H), 7.11 (d, J=7.5 Hz, 1H), 6.10 (d, J=14.1 Hz, 2H), 3.72 (s, 6H), 2.73 (t, J=6.6 Hz, 4H), 2.15 (s, 3H), 2.12-2.02 (m, 2H), 1.42 (s, 12H). ¹³C NMR (101 MHz, CDCl3) δ 173.51, 161.67, 146.21, 140.25, 138.23, 136.07, 133.10, 131.80, 131.31,130.70, 130.47, 130.19, 129.37, 128.73, 128.02, 127.62, 126.31, 124.83, 121.99, 110.70, 99.87, 50.35, 32.12, 27.32, 27.12, 24.75, 21.39, 18.96. HRMS (ESI+) calcd 639.3734, found 639.3698 for C₄₇H₄₇N₂+ (M+).

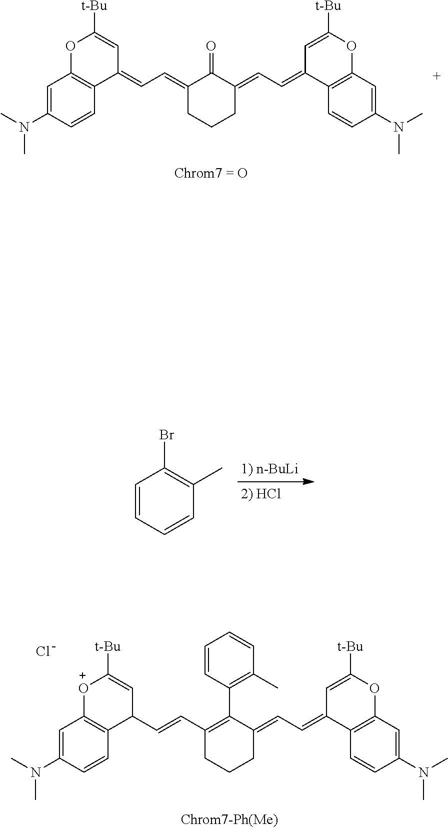

2-(tert-Butyl)-4-(2-(6-(2-(2-(tert-butyl)-7-(dimethylamino)-4H-chromen-4-ylidene)ethylidene)-2′-methyl-3,4,5, 6-tetrahydro-[1,1′-biphenyl]-2-yl)vinyl)-7-(dimethylamino)chromenylium chloride [Chrom7-Ph(Me)]: Following General Procedure B, Chrom7═O (10 mg, 0.017 mmol) was reacted with 2-bromotulene (35 mg, 0.20 mmol) and n-BuLi (2.3 M, 0.06 mL, 0.14 mmol) to give Chrom7-Ph(Me) as a dark purple solid (7.6 mg, 63%). ¹H NMR (400 MHz, MeOD) δ 7.92 (d, J=9.3 Hz, 2H), 7.49-7.33 (m, 3H), 7.13 (d, J=7.3 Hz, 1H), 7.06 (d, J=13.7 Hz, 2H), 6.94 (dd, J=9.3, 2.6 Hz, 2H), 6.85 (d, J=13.8 Hz, 2H), 6.54 (d, J=2.5 Hz, 2H), 6.05 (s, 2H), 3.11 (s, 12H), 2.13 (s, 3H), 2.11-1.98 (m, 2H), 1.20 (s, 18H). ¹³C NMR (101 MHz, MeOD) δ 169.35, 158.75, 156.31, 154.49, 145.27, 141.95, 137.98, 133.39, 130.51, 129.66, 127.93, 125.29, 125.18, 117.59, 112.75, 111.51, 109.74, 98.37, 96.89, 39.02, 36.01, 26.74, 24.94, 21.44, 18.14. HRMS (ESI+) calcd 679.4258, found 679.4250 for C₄₇H₅₅N₂O₂+ (M+).

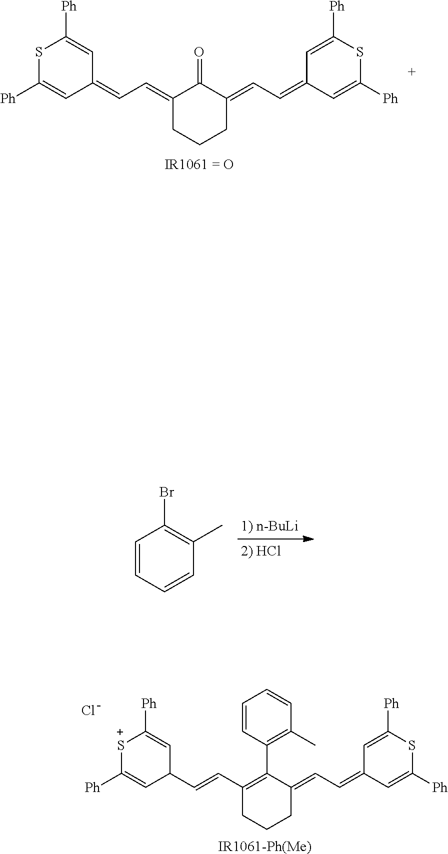

4-(2-(6-(2-(2,6-diphenyl-4H-thiopyran-4-ylidene)ethylidene)-2′-methyl-3,4,5,6-tetrahydro-[1,1′-biphenyl]-2-yl)vinyl)-2,6- diphenylthiopyrylium chloride [IR1061-Ph(Me)]: Following General Procedure B, IR1061═O (28 mg, 0.043 mmol) was reacted with 2-bromotulene (88 mg, 0.52 mmol) and n-BuLi (2.3 M, 0.15 mL, 0.35 mmol) to give IR1061-Ph(Me) as a dark purple solid (17 mg, 56%). ¹H NMR (400 MHz, CDCl3 and MeOD) δ 7.62-7.39 (m, 20H), 7.39-7.28 (m, 7H), 7.14-7.02 (m, 3H), 6.66 (d, J=14.0 Hz, 2H), 2.88 (s, 6H), 2.83-2.64 (m, 4H), 2.09 (s, 3H), 2.07-1.90 (m, 2H). ¹³C NMR (101 MHz, CDCl3 and MeOD) δ 135.68, 131.46, 130.67, 130.16, 129.65, 128.23, 126.59, 125.60, 124.41, 123.02, 25.50, 21.37, 19.48. HRMS (ESI+) calcd 717.2644, found 717.2683 for C₅₁H₄₁S₂+ (M+H+).

General procedure C: preparation of meso-substituted heptamethine dyes. Unless otherwise noted, aryl bromide (0.34 mmol) was dissolved in dry THF (1 mL) and cooled to −84° C. To this solution was added t-BuLi (1.7 M in pentane, 0.40 mL, 0.68 mmol). The mixture was stirred for 40 min at this temperature. A red to orange solution of keto-heptamethine (0.042 mmol) in dry THF (1 mL) was added dropwise. The mixture was allowed to warm up to room temperature and stirred for 30 min. The yellow or orange reaction mixture was quenched by adding 1:10 HCl which resulted in a rapid color change into dark green. The mixture was diluted in H₂O and extracted with CH₂Cl₂ (×4) and dried (Na₂SO₄). The crude product was purified by column chromatography (1:30 to 1:20 methanol/CH₂Cl₂).

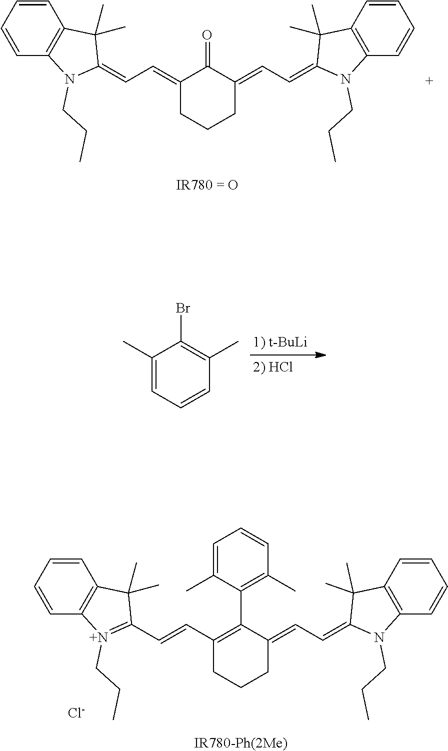

2-(2-(6-(2-(3,3-Dimethyl-1-propylindolin-2-ylidene)ethylidene)-2′,6′-dimethyl-3,4,5,6-tetrahydro-[1,1′-biphenyl]-2-yl)vinyl)- 3,3-dimethyl-1-propyl-3H-indol-1-ium chloride [IR780-Ph(2Me)]: Following General Procedure C, IR780═O (22 mg, 0.042 mmol) was reacted with 2-bromo-1,3-dimethylbenzene (62 mg, 0.34 mmol) and t-BuLi (1.7 M, 0.40 mL, 0.68 mmol) to give IR780-Ph(2Me) as a green solid film (20 mg, 75%). 1H NMR (400 MHz, MeOD) δ 7.42-7.28 (m, 7H), 7.28-7.21 (m, 4H), 7.18 (t, J=7.5 Hz, 2H), 6.21 (d, J=14.1 Hz, 2H), 4.06 (t, J=7.4 Hz, 4H), 2.75 (s, 4H), 2.12 (s, 6H), 2.10-2.05 (m, 2H), 1.83 (h, J=7.3 Hz, 4H), 1.15 (s, 12H), 1.02 (t, J=7.4 Hz, 6H). ¹³C NMR (101 MHz, MeOD) δ 173.46, 162.69, 146.98, 143.78, 142.22, 138.95, 137.16, 129.70, 129.50, 129.13, 125.92, 123.34, 111.86, 101.06, 49.93, 46.37, 28.19, 25.40, 22.60, 21.72, 19.42, 14.43, 11.65. HRMS (ESI+) calcd 609.4203, found 609.4196 for C₄₄H₅₃N₂+ (M+).

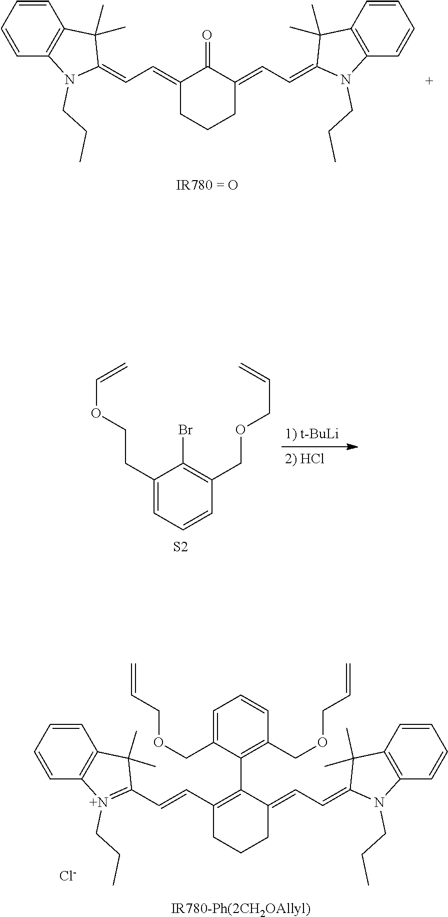

2-(2-(2′,6′-Bis((allyloxy)methyl)-6-(2-(3,3-dimethyl-1-propylindolin-2-ylidene)ethylidene)-3,4,5,6-tetrahydro-[1,1′-biphenyl]-2- yl)vinyl)-3,3-dimethyl-1-propyl-3H-indol-1-ium chloride [IR780-Ph(2CH2OAllyl)]: Following General Procedure C, IR780═O (22 mg, 0.042 mmol) was reacted with S2 (100 mg, 0.34 mmol) and t-BuLi (1.7 M, 0.40 mL, 0.68 mmol) to give IR780-Ph(2CH2OAllyl) as a green solid film (29 mg, 91%). ¹H NMR (400 MHz, MeOD) δ 7.71-7.65 (m, 2H), 7.65-7.59 (m, 1H), 7.39-7.29 (m, 4H), 7.26-7.14 (m, 6H), 6.19 (d, J=14.0 Hz, 2H), 5.83-5.69 (m, 2H), 5.16 (dd, J=17.3, 1.8 Hz, 2H), 5.05 (dd, J=10.4, 1.6 Hz, 2H), 4.28 (s, 4H), 4.06 (t, J=7.3 Hz, 4H), 3.91 (dt, J=5.6, 1.5 Hz, 4H), 2.75 (t, J=6.2 Hz, 4H), 2.10 (p, J=6.2 Hz, 2H), 1.82 (h, J=7.4 Hz, 4H), 1.15 (s, 12H), 1.01 (t, J=7.4 Hz, 6H). ¹³C NMR (101 MHz, MeOD) δ 173.55, 159.07, 148.22, 143.72, 142.26, 138.10, 137.88, 136.05, 131.82, 129.86, 129.75, 129.66, 125.97, 123.30, 117.23, 111.88, 101.07, 72.97, 70.63, 49.98, 49.85, 46.34, 28.18, 25.56, 22.58, 21.74, 11.66. HRMS (ESI+) calcd 721.4728, found 721.4721 for C₅₀H₆₁N₂O₂+ (M+).

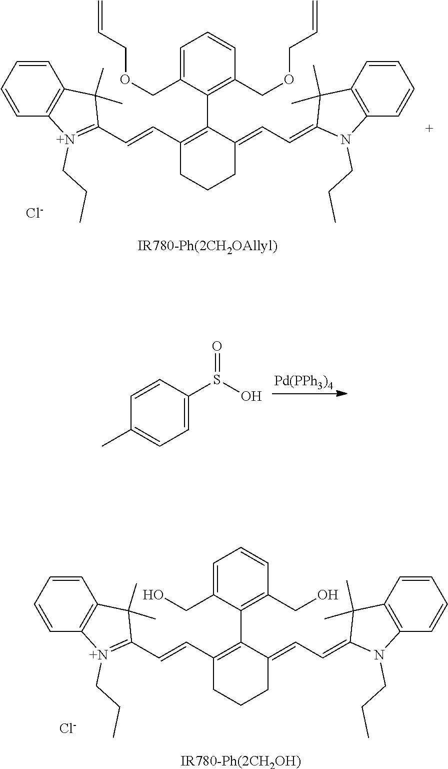

2-(2-6-(2-(3,3-Dimethyl-1-propylindolin-2-ylidene)ethylidene)-2′,6′-bis(hydroxymethyl)-3,4,5,6-tetrahydro-[1,1′-biphenyl]-2- yl)vinyl)-3,3-dimethyl-1-propyl-3H-indol-1-ium chloride [IR780-Ph(2CH2OH)]: IR780-Ph(2CH2OAllyl) (22 mg, 0.029 mmol), p-toluenesulfinic acid (36 mg, 0.23 mmol) and Pd(PPh3)4 (6.7 mg, 0.0058 mmol) was dissolved in ethanol (2 mL) followed by three freeze-pump-thaw cycles. The mixture was stirred at 65° C. under N₂ for 4.5 h. The reaction was diluted in CH₂Cl₂, washed with sat. NaHCO₃ and dired (Na₂SO₄). The crude product was purified by column chromatography (1:8 methanol/CH₂Cl₂) to give IR780-Ph(2CH₂OH) as a green solid film (13 mg, 66%). 1H NMR (400 MHz, MeOD) 0 7.74-7.69 (m, 2H), 7.67-7.61 (m, 1H), 7.39-7.29 (m, 4H), 7.27-7.13 (m, 6H), 6.19 (d, J=14.1 Hz, 2H), 4.43 (s, 4H), 4.05 (t, J=7.3 Hz, 4H), 2.74 (t, J=6.2 Hz, 4H), 2.09 (t, J=5.8 Hz, 2H), 1.82 (h, J=7.4 Hz, 4H), 1.16 (s, 12H), 1.01 (t, J=7.4 Hz, 6H). HRMS (ESI+) calcd 641.4102, found 641.4109 for C₄₄H₅₃N₂O₂+ (M+).

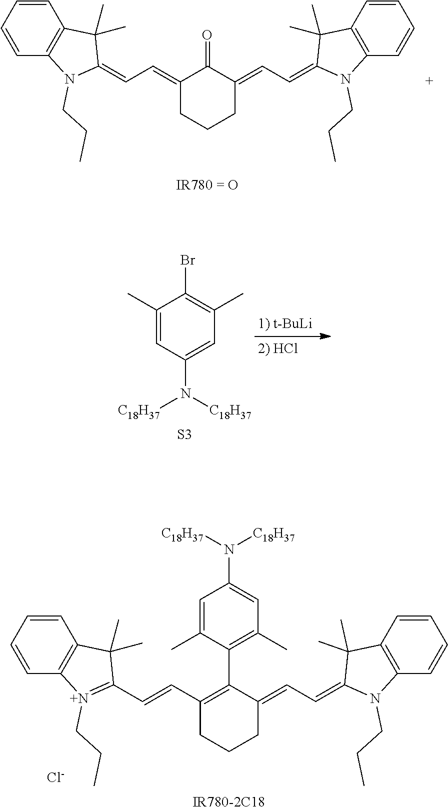

2-(2-(6-(2-(3,3-dimethyl-1-propylindolin-2-ylidene)ethylidene)-4′-(dioctadecylamino)-2′,6′-dimethyl-3,4,5,6-tetrahydro-[1,1′- biphenyl]-2-yl)vinyl)-3,3-dimethyl-1-propyl-3H-indol-1-ium chloride (IR780-2C18): Following General Procedure C, t-BuLi (1.7 M, 0.40 mL, 0.68 mmol) was added to S3 (238 mg, 0.34 mmol) in THE solution and warmed up to room temperature until all solids dissolved, at which point IR780═O (22 mg, 0.042 mmol) was added and stirred for 15 min. Subsequent work-up and column chromatograph gave IR780-2C18 as a green solid (25 mg, 51%). ¹H NMR (500 MHz, CDCl3) δ 7.35-7.27 (m, 4H), 7.18-7.04 (m, 6H), 6.49 (s, 2H), 6.04 (d, J=14.1 Hz, 2H), 4.00 (t, J=7.3 Hz, 4H), 3.33 (t, J=7.3 Hz, 4H), 2.66 (t, J=5.9 Hz, 4H), 2.03 (p, J=5.3 Hz, 2H), 1.98 (s, 6H), 1.84 (h, J=7.3 Hz, 4H), 1.65-1.56 (m, 4H), 1.28 (d, J=69.3 Hz, 64H), 1.16 (s, 12H), 1.02 (t, J=7.4 Hz, 6H), 0.84 (t, J=6.8 Hz, 6H). ¹³C NMR (126 MHz, CDCl3) δ 171.79, 164.06, 146.57, 142.80, 141.04, 136.54, 131.76, 128.78, 124.66, 121.98, 112.47, 110.73, 100.22, 51.46, 48.79, 46.18, 32.00, 29.90-29.72 (m), 29.40, 28.01, 27.55, 27.38, 24.87, 22.72, 21.60, 20.88, 19.97, 14.05, 11.65. HRMS (ESI+) calcd 1128.9946, found 1128.9935 for C₈₀H₁₂₆N₃+ (M+).

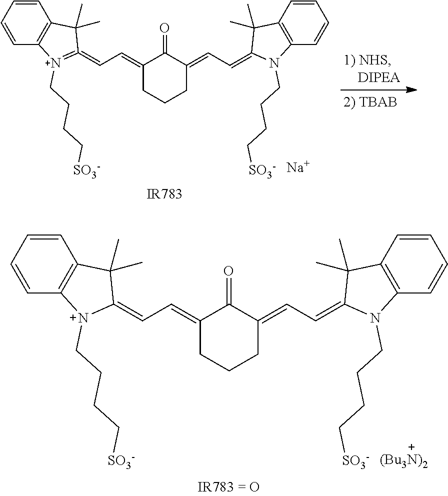

Tetrabutylammonium 4,4′-(((2-oxocyclohexane-1,3-diylidene)bis(ethane-2,1-diylidene))bis(3,3-dimethylindoline-1-yl-2-ylidene))bis(butane-1-sulfonate) (IR783═O tetrabutylammonium salt): Following General Procedure B, IR783 (80 mg, 0.11 mmol) was reacted with NHS (37 mg, 0.32 mmol) and DIPEA (56 μL, 0.32 mmol) in DMF (3 mL) for 3.5 h to afford a red orange solution. The mixture was diluted with H₂O (6 mL) and loaded onto preparative HPLC (Phenomenex Kinetex 5 μm phenyl-hexyl, 250×21.2 mm). The column was flushed three times with tetrabutylammonium bromide solution (7 mL, 0.2 M dissolved in 30% methanol in H2O) and separated with a gradient of 30%-90% methanol to give IR783═O tetrabutylammonium salt as a dark, red orange solid (84 mg, 66%). ¹H NMR (500 MHz, MeOD) δ 8.19 (d, J=12.7 Hz, 2H), 7.26 (d, J=7.5 Hz, 2H), 7.21 (t, J=7.8 Hz, 2H), 7.04-6.81 (m, 4H), 5.61 (d, J=13.3 Hz, 2H), 3.83 (t, J=6.9 Hz, 4H), 3.23 (t, J=8.6 Hz, 16H), 2.87 (t, J=7.6 Hz, 4H), 2.62 (t, J=6.4 Hz, 4H), 2.03-1.81 (m, 10H), 1.69-1.60 (m, 28H), 1.41 (h, J=7.7 Hz, 16H), 1.01 (t, J=7.6 Hz, 24H). ¹³C NMR (126 MHz, MeOD) δ 188.52, 165.19, 145.28, 140.81, 136.15, 129.01, 127.14, 122.74, 122.16, 108.65, 94.00, 59.57, 52.30, 47.90, 43.33, 29.10, 26.69, 26.56, 24.79, 23.82, 23.72, 20.67, 13.91. HRMS (ESI−) calcd 707.2830, found 707.2829 for C₃₈H₄₇N₂O₇S₂− [M2−+H+].

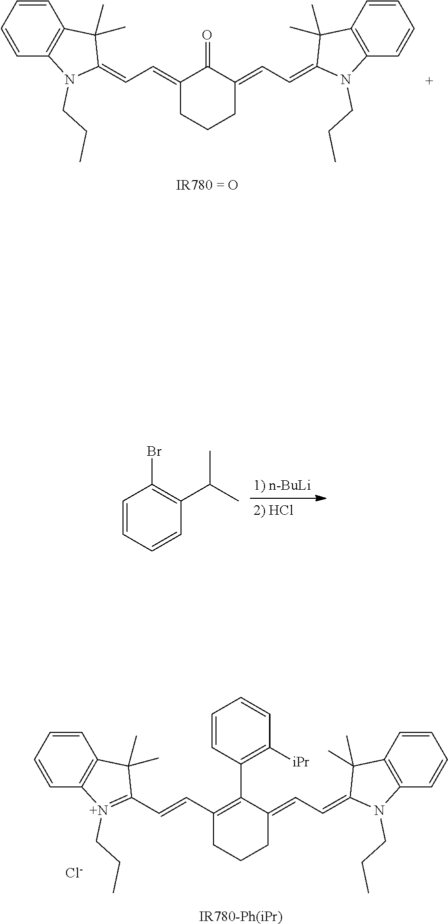

2-(2-(6-(2-(3,3-dimethyl-1-propylindolin-2-ylidene)ethylidene)-2′-isopropyl-3,4,5,6-tetrahydro-[1,1′-biphenyl]-2-yl)vinyl)-3,3-dimethyl-1-propyl-3H-indol-1-ium chloride [IR780-Ph(iPr)]: Following General Procedure B, IR780═O (22 mg, 0.042 mmol) was reacted with 1-bromo-2-isopropylbenzene (101 mg, 0.51 mmol) and n-BuLi (2.3 M, 0.15 mL, 0.34 mmol) to give IR780-Ph(iPr) as a green solid film (21 mg, 75%). ¹H NMR (500 MHz, CDCl3) δ 7.50 (d, J=3.0 Hz, 2H), 7.39-7.24 (m, 3H), 7.19-7.10 (m, 6H), 7.06 (d, J=7.7 Hz, 2H), 7.00 (d, J=7.2 Hz, 1H), 6.06 (d, J=14.0 Hz, 2H), 4.01 (t, J=6.4 Hz, 4H), 2.83-2.51 (m, 5H), 2.19-2.12 (m, 2H), 1.83 (q, J=7.0 Hz, 4H), 1.15 (s, 6H), 1.08 (d, J=6.1 Hz, 12H), 1.01 (t, J=7.2 Hz, 6H). ¹³C NMR (126 MHz, CDCl3) δ 172.00, 162.16, 148.38, 147.56, 142.70, 141.02, 137.12, 132.39, 129.55, 129.06, 128.76, 126.47, 125.90, 124.94, 122.06, 110.83, 100.32, 48.85, 46.33, 30.67, 29.74, 28.16, 27.79, 25.15, 24.53, 21.59, 20.95, 11.59. HRMS (ESI+) calcd 623.4360, found 623.4362 for C₄₅H₅₅N₂+ (M+).

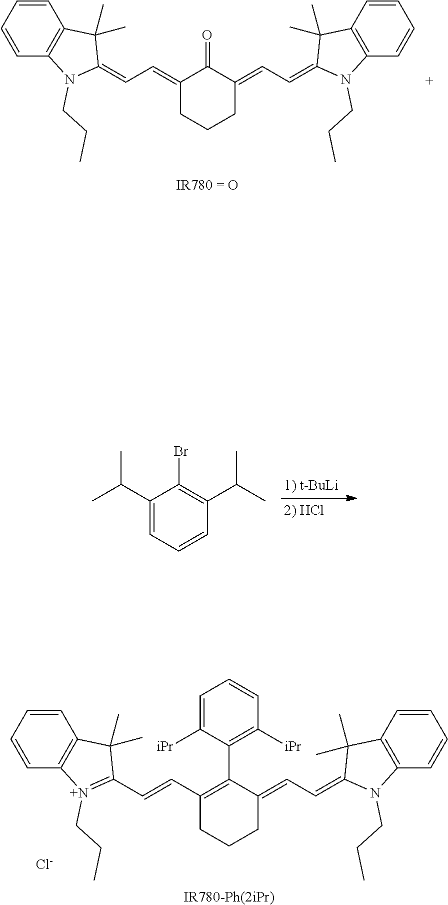

2-(2-(6-(2-(3,3-dimethyl-1-propylindolin-2-ylidene)ethylidene)-2′,6′-diisopropyl-3,4,5,6-tetrahydro-[1,1′-biphen yl]-2- yl)vinyl)-3,3-dimethyl-1-propyl-3H-indol-1-ium chloride [IR780-Ph(2iPr)]: Following General Procedure C, IR780═O (22 mg, 0.042 mmol) was reacted with 2-bromo-1,3-diisopropylbenzene (82 mg, 0.34 mmol) and t-BuLi (1.7 M, 0.40 mL, 0.68 mmol). The mixture was purified by column chromatography followed by semi-preparative HPLC (Phenomenex Gemini 5 μm C18, 250×10.0 mm, 65%-100% MeCN in water with 0.1% TFA) to give IR780-Ph(2iPr) as a green solid (9.2 mg, 31%). ¹H NMR (400 MHz, MeOD) δ 7.56 (t, J=7.8 Hz, 1H), 7.43 (d, J=7.8 Hz, 2H), 7.39-7.31 (m, 4H), 7.28-7.16 (m, 6H), 6.21 (d, J=14.2 Hz, 2H), 4.06 (t, J=7.3 Hz, 4H), 2.85 (p, J=6.8 Hz, 2H), 2.78 (t, J=6.3 Hz, 4H), 2.11 (p, J=6.5 Hz, 2H), 1.83 (h, J=7.4 Hz, 4H), 1.21 (s, 12H), 1.14 (d, J=6.8 Hz, 12H), 1.01 (t, J=7.4 Hz, 6H). ¹³C NMR (101 MHz, MeOD) δ 173.23, 162.07, 148.07, 147.77, 143.75, 141.99, 136.34, 132.75, 130.35, 129.73, 126.03, 124.88, 123.29, 111.90, 101.12, 50.03, 46.42, 32.26, 28.63, 25.69, 25.45, 22.76, 21.72, 11.62. HRMS (ESI+) calcd 665.4829, found 665.4825 for C₄₈H₆₁N₂+ (M+).

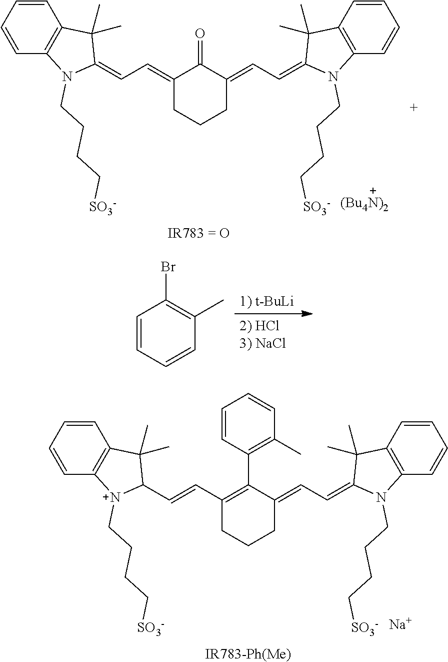

Sodium 4-(2-(2-(6-(2-(3,3-dimethyl-1-(4-sulfonatobutyl)-3H-indol-1-ium-2-yl)vinyl)-2′-methyl-4,5-dihydro-[1,1′- biphenyl]-2 (3H)-ylidene)ethylidene)-3,3-dimethylindolin-1-yl)butane-1-sulfonate [IR783-Ph(Me)]: Following General Procedure C, IR783═O tetrabutylammonium salt (41 mg, 0.034 mmol) dissolved in dry THF (3 mL) was reacted with 2-bromotoluene (70.6 mg, 0.41 mmol) and t-BuLi (1.7 M, 0.49 mL, 0.83 mmol) in dry THF (4 mL). After stirring at room temperature for 30 min, the reaction was quenched by adding excess amount of acetic acid. The mixture was concentrated, diluted with H2O (6 mL), extracted with hexanes (×2), and loaded onto preparative HPLC (Phenomenex Kinetex 5 μm phenyl-hexyl, 250×21.2 mm). The column was flushed three times with NaCl solution (7 mL, 0.2 M dissolved in 45% methanol in H₂O) and separated with a gradient of 45%-90% methanol to give IR783-Ph(Me) sodium salt as a green solid (17 mg, 60%). 1H NMR (500 MHz, MeOD) δ 7.51-7.46 (m, 2H), 7.46-7.40 (m, 1H), 7.37-7.29 (m, 4H), 7.26 (d, J=8.2 Hz, 2H), 7.21 (d, J=14.0 Hz, 2H), 7.16 (t, J=7.4 Hz, 2H), 7.10 (d, J=7.6 Hz, 1H), 6.23 (d, J=14.0 Hz, 2H), 4.11 (t, J=6.9 Hz, 4H), 2.88 (t, J=6.2 Hz, 4H), 2.75 (t, J=6.6 Hz, 4H), 2.14 (s, 3H), 2.07 (p, J=6.3 Hz, 2H), 1.99-1.88 (m, 8H), 1.16 (s, 6H), 1.13 (s, 6H). ¹³C NMR (126 MHz, MeOD) δ 173.31, 163.39, 148.60, 143.68, 142.22, 139.74, 137.40, 132.25, 131.66, 130.62, 129.73, 129.65, 127.42, 125.86, 123.23, 111.87, 101.06, 51.86, 44.81, 28.21, 28.03, 27.20, 25.52, 23.61, 22.63, 19.07. HRMS (ESI−) calcd 781.3344, found 781.3351 for C₄₅H₅₃N₂O₆S₂− (M−).

Future Developments: The synthetic method here will be further expanded from single 4′-modification to incorporate two aryl modifications on 4′ and 3′ site of heptamethine fluorophores to furnish even more (photo) stable fluorophores. We will also install click reaction handles on ortho-positions of the 4′-aryl ring to introduce more complex conjugation by click reaction with even higher hydrophilicity and steric demand that facilitate the solubility of the heptamethine fluorophores.

Advantages: This conversion is compatible with most existing hydrophobic heptamethine fluorophores with a center chloride modification, which is a common scaffold in commercial and reported near-infrared and shortwave infrared fluorophores. After the simple two-step conversion, the fluorophore can achieve higher brightness, much enhanced stability in physiological condition, higher photostability, and most importantly, water solubility when appropriate substituents are introduced with this method. This will bypass all the formulation preparation for animal imaging, affording more reliable imaging results. Adding bioconjugation handle is also possible with this design.

Summary: Optical imaging with near-infrared (NIR, 700-1000 nm) and shortwave infrared (SWIR, 1000-2000 nm) light is receiving a growing attention for its potential biomedical use, but its application falls short when compared to wide-spread usage of established imaging modalities such as CT, MRI and PET. As the instrument for optical imaging, particularly SWIR cameras, are getting more popularized, the lack of contrast agents, or NIR/SWIR fluorophores, has emerged as a limiting step for the widespread clinical use of this imaging modality. Despite the ever increasing literature reports of such fluorophores, clinical NIR/SWIR imaging still largely rely on decades-old indocyanine green (ICG). Such discrepancy largely comes from the limited solubility and stability of the fluorophores. Indeed, a major portion of reported NIR and SWIR fluorophores are administrated as formulations, which can lead to complications in vivo, whereas the water-soluble dyes can face degradation issues in aqueous physiological condition. As such, there is a critical need to revamp building blocks for NIR/SWIR fluorophore synthesis to produce water-soluble and stable contrast agents, which will greatly facilitate the development of clinical optical imaging for their wide-spread use in cost-effective screens, monitoring treatments or guided surgeries.

In this invention, we have come up with the lithium addition reaction that easily convert widely available center (4′)-chloride heptamethine dyes into center-aryl substituted fluorophores as new NIR and SWIR fluorophores. The newly introduced center aryl ring, especially the ortho-disubstituted aryl ring, can carry hydrophilic functionalities and high steric demand, which greatly enhances the water solubility and stability of the resulting dyes. Our synthetic strategy is particularly useful as ortho-disubstituted aryl ring cannot be easily installed at 4′-position of heptamethine otherwise: existing methods with Suzuki coupling on 4′-chloro heptamethine dyes can only afford substitution product with ortho-unsubstituted aryl rings or ortho-carboxylphenyl, whereas di-substituted counterparts can only be previously accessed from laborious de novo synthesis from tailored linker building blocks. Thus, our method stands out by offering a mild, efficient alternative for incorporating challenging 4′-aryl substitutions as a post-synthetic modification into heptamethine fluorophore scaffolds.

### References

(1) Bricks, J. L.; Kachkovskii, A. D.; Slominskii, Y. L.; Gerasov, A. O.; Popov, S. V. Molecular Design of near Infrared Polymethine Dyes: A Review. Dyes Pigm. 2015, 121, 238-255. https://doi.org/10.1016/j.dyepig.2015.05.016.
(2) Alander, J. T.; Kaartinen, I.; Laakso, A.; Pätilä, T.; Spillmann, T.; Tuchin, V. V.; Venermo, M.; Välisuo, P. A Review of Indocyanine Green Fluorescent Imaging in Surgery. Journal of Biomedical Imaging 2012, 2012, 7:7. https://doi.org/10.1155/2012/940585.
(3) Mahalingam, S. M.; Kularatne, S. A.; Myers, C. H.; Ga-gare, P.; Norshi, M.; Liu, X.; Singhal, S.; Low, P. S. Evaluation of Novel Tumor-Targeted Near-Infrared Probe for Fluorescence-Guided Surgery of Cancer. J. Med. Chem. 2018, 61(21), 9637-9646. https://doi.org/10.1021/acs.jmedchem.8b01115.
(4) Randall, L. M.; Wenham, R. M.; Low, P. S.; Dowdy, S. C.; Tanyi, J. L. A Phase II, Multicenter, Open-Label Trial of OTL38 Injection for the Intra-Operative Imaging of Folate Receptor-Alpha Positive Ovarian Cancer. Gynecologic Oncology 2019, 155(1), 63-68. https://doi.org/10.1016/j.ygyno.2019.07.010.
(5) Zhu, S.; Tian, R.; Antaris, A. L.; Chen, X.; Dai, H. Near-Infrared-II Molecular Dyes for Cancer Imaging and Surgery. Advanced Materials 2019, 31(24), 1900321. https://doi.org/10.1002/adma.201900321.
(6) Lei, Z.; Zhang, F. Molecular Engineering of NIR-II Fluorophores for Improved Biomedical Detection. Angewandte Chemie International Edition 2021, 60(30), 16294-16308. https://doi.org/10.1002/anie.202007040.
(7) Zhao, X.; Zhang, F.; Lei, Z. The Pursuit of Polymethine Fluorophores with NIR-II Emission and High Brightness for in Vivo Applications. Chem. Sci. 2022, 13(38), 11280-11293. https://doi.org/10.1039/D2SC03136A.
(8) Wong, K. C. Y.; Sletten, E. M. Extending Optical Chemical Tools and Technologies to Mice by Shifting to the Shortwave Infrared Region. Current Opinion in Chemical Biology 2022, 68, 102131. https://doi.org/10.1016/j.cbpa.2022.102131.
(9) Luciano, M. P.; Crooke, S. N.; Nourian, S.; Dingle, I.; Nani, R. R.; Kline, G.; Patel, N. L.; Robinson, C. M.; Difilippantonio, S.; Kalen, J. D.; Finn, M. G.; Schnermann, M. J. A Nonaggregating Heptamethine Cyanine for Building Brighter Labeled Biomolecules. ACS Chem. Biol. 2019, 14(5), 934-940. https://doi.org/10.1021/acschembio.9b00122.
(10) Li, D.-H.; Gamage, R. S.; Smith, B. D. Sterically Shielded Hydrophilic Analogs of Indocyanine Green. J. Org. Chem. 2022, 87(17), 11593-11601. https://doi.org/10.1021/acs.joc.2c01229.
(11) Jia, S.; Lin, E. Y.; Mobley, E. B.; Lim, I.; Guo, L.; Kallepu, S.; Low, P. S.; Sletten, E. M. Water-Soluble Chromenylium Dyes for Shortwave Infrared Imaging in Mice. Chem 2023, 9(12), 3648-3665. https://doi.org/10.1016/j.chempr.2023.08.021.
(12) Li, D.-H.; Gamage, R. S.; Oliver, A. G.; Patel, N. L.; Muhammad Usama, S.; Kalen, J. D.; Schnermann, M. J.; Smith, B. D. Doubly Strapped Zwitterionic NIR-I and NIR-II Heptamethine Cyanine Dyes for Bioconjugation and Fluorescence Imaging. Angewandte Chemie International Edition 2023, 62(28), e202305062. https://doi.org/10.1002/anie.202305062.
(13) Gorka, A. P.; Schnermann, M. J. Harnessing Cyanine Photooxidation: From Slowing Photobleaching to near-IR Un-caging. Current Opinion in Chemical Biology 2016, 33, 117-125. https://doi.org/10.1016/j.cbpa.2016.05.022.
(14) Demchenko, A. P. Photobleaching of Organic Fluorophores: Quantitative Characterization, Mechanisms, Protection *. Methods Appl. Fluoresc. 2020, 8(2), 022001. https://doi.org/10.1088/2050-6120/ab7365.
(15) Li, D.-H.; Smith, B. D. Deuterated Indocyanine Green (ICG) with Extended Aqueous Storage Shelf-Life: Chemical and Clinical Implications. Chemistry 2021, 27 (58), 14535-14542. https://doi.org/10.1002/chem.202102816.
(16) Mindt, S.; Karampinis, I.; John, M.; Neumaier, M.; Nowak, K. Stability and Degradation of Indocyanine Green in Plasma, Aqueous Solution and Whole Blood. Photochem Photobiol Sci 2018, 17(9), 1189-1196. https://doi.org/10.1039/c8pp00064f.
(17) Medeiros, N. G.; Braga, C. A.; Câmara, V. S.; Duarte, R. C.; Rodembusch, F. S. Near-Infrared Fluorophores Based on Heptamethine Cyanine Dyes: From Their Synthesis and Photo-physical Properties to Recent Optical Sensing and Bioimaging Applications. Asian Journal of Organic Chemistry 2022, 11(6), e202200095. https://doi.org/10.1002/ajoc.202200095.
(18) Strekowski, L.; Lipowska, M.; Patonay, G. Substitution Reactions of a Nucleofugal Group in Heptamethine Cyanine Dyes. Synthesis of an Isothiocyanato Derivative for Labeling of Proteins with a near-Infrared Chromophore. J. Org. Chem. 1992, 57(17), 4578-4580. https://doi.org/10.1021/jo00043a009.
(19) Oushiki, D.; Kojima, H.; Terai, T.; Arita, M.; Hanaoka, K.; Urano, Y.; Nagano, T. Development and Application of a Near-Infrared Fluorescence Probe for Oxidative Stress Based on Differential Reactivity of Linked Cyanine Dyes. J. Am. Chem. Soc. 2010, 132(8), 2795-2801. https://doi.org/10.1021/ja910090v.
(20) Smith, B. A.; Akers, W. J.; Leevy, W. M.; Lampkins, A. J.; Xiao, S.; Wolter, W.; Suckow, M. A.; Achilefu, S.; Smith, B. D. Optical Imaging of Mammary and Prostate Tumors in Living Animals Using a Synthetic Near Infrared Zinc(II)-Dipicolylamine Probe for Anionic Cell Surfaces. J. Am. Chem. Soc. 2010, 132(1), 67-69. https://doi.org/10.1021/ja908467y.
(21) Samanta, A.; Vendrell, M.; Das, R.; Chang, Y.-T. Development of Photostable Near-Infrared Cyanine Dyes. Chem. Commun. 2010, 46(39), 7406-7408. https://doi.org/10.1039/C0CC02366C.
(22) Choi, H. S.; Nasr, K.; Alyabyev, S.; Feith, D.; Lee, J. H.; Kim, S. H.; Ashitate, Y.; Hyun, H.; Patonay, G.; Strekowski, L.; Henary, M.; Frangioni, J. V. Synthesis and In Vivo Fate of Zwitterionic Near-Infrared Fluorophores. Angewandte Chemie International Edition 2011, 50(28),6258-6263. https://doi.org/10.1002/anie.201102459.
(23) Choi, H. S.; Gibbs, S. L.; Lee, J. H.; Kim, S. H.; Ashitate, Y.; Liu, F.; Hyun, H.; Park, G.; Xie, Y.; Bae, S.; Henary, M.; Frangioni, J. V. Targeted Zwitterionic Near-Infrared Fluorophores for Improved Optical Imaging. Nat Biotechnol 2013, 31(2), 148-153. https://doi.org/10.1038/nbt.2468.
(24) Lee, H.; Mason, J. C.; Achilefu, S. Heptamethine Cya-nine Dyes with a Robust C—C Bond at the Central Position of the Chromophore. J. Org. Chem. 2006, 71(20), 7862-7865. https://doi.org/10.1021/jo061284u.
(25) König, S. G.; Krämer, R. Accessing Structurally Diverse Near-Infrared Cyanine Dyes for Folate Receptor-Targeted Cancer Cell Staining. Chemistry—A European Journal 2017, 23(39), 9306-9312. https://doi.org/10.1002/chem.201700026.
(26) Lee, H.; Mason, J. C.; Achilefu, S. Synthesis and Spectral Properties of Near-Infrared Aminophenyl-, Hydroxyphenyl-, and Phenyl-Substituted Heptamethine Cyanines. J. Org. Chem. 2008, 73(2), 723-725. https://doi.org/10.1021/jo701793h.
(27) Levitz, A.; Marmarchi, F.; Henary, M. Introduction of Various Substitutions to the Methine Bridge of Heptamethine Cyanine Dyes Via Substituted Dianil Linkers. Photochem. Photobiol. Sci. 2018, 17(10), 1409-1416. https://doi.org/10.1039/C8PP00218E.
(28) Štacková, L.; Štacko, P.; Klán, P. Approach to a Substituted Heptamethine Cyanine Chain by the Ring Opening of Zincke Salts. J. Am. Chem. Soc. 2019, 141(17), 7155-7162. https://doi.org/10.1021/jacs.9b02537.
(29) Usama, S. M.; Marker, S. C.; Li, D.-H.; Caldwell, D. R.; Stroet, M.; Patel, N. L.; Tebo, A. G.; Hernot, S.; Kalen, J. D.; Schnermann, M. Method To Diversify Cyanine Chromophore Functionality Enables Improved Biomolecule Tracking and Intracellular Imaging. J. Am. Chem. Soc. 2023, 145(27), 14647-14659. https://doi.org/10.1021/jacs.3c01765.
(30) Wang, S.; Fan, Y.; Li, D.; Sun, C.; Lei, Z.; Lu, L.; Wang, T.; Zhang, F. Anti-Quenching NIR-II Molecular Fluorophores for in Vivo High-Contrast Imaging and PH Sensing. Nat Commun 2019, 10(1), 1058. https://doi.org/10.1038/s41467-019-09043-x.
(31) Pengshung, M.; Li, J.; Mukadum, F.; Lopez, S. A.; Slet-ten, E. M. Photophysical Tuning of Shortwave Infrared Flavylium Heptamethine Dyes via Substituent Placement. Org Lett 2020, 22(15), 6150-6154. https://doi.org/10.1021/acs.orglett.0c02213.
(32) Cosco, E. D.; Spearman, A. L.; Ramakrishnan, S.; Lingg, J. G. P.; Saccomano, M.; Pengshung, M.; Arús, B. A.; Wong, K. C. Y.; Glasl, S.; Ntziachristos, V.; Warmer, M.; Mclaughlin, R. R.; Bruns, O. T.; Sletten, E. M. Shortwave Infrared Polymethine Fluorophores Matched to Excitation Lasers Enable Non-Invasive, Multicolour in Vivo Imaging in Real Time. Nature Chemistry 2020, 12(12), 1123-1130. https://doi.org/10.1038/s41557-020-00554-5.
(33) Cosco, E. D.; Arús, B. A.; Spearman, A. L.; Atallah, T. L.; Lim, I.; Leland, O. S.; Caram, J. R.; Bischof, T. S.; Bruns, O. T.; Sletten, E. M. Bright Chromenylium Polymethine Dyes Enable Fast, Four-Color In Vivo Imaging with Shortwave Infrared Detection. J. Am. Chem. Soc. 2021, 143(18), 6836-6846. https://doi.org/10.1021/jacs.0c11599.
(34) Yang, Y.; Sun, C.; Wang, S.; Yan, K.; Zhao, M.; Wu, B.; Zhang, F. Counterion-Paired Bright Heptamethine Fluorophores with NIR-II Excitation and Emission Enable Multiplexed Biomedical Imaging. Angewandte Chemie International Edition 2022, 61(24), e202117436. https://doi.org/10.1002/anie.202117436.
(35) Urano, Y.; Kamiya, M.; Kanda, K.; Ueno, T.; Hirose, K.; Nagano, T. Evolution of Fluorescein as a Platform for Finely Tunable Fluorescence Probes. J Am Chem Soc 2005, 127(13), 4888-4894. https://doi.org/10.1021/ja043919h.
(36) Egawa, T.; Koide, Y.; Hanaoka, K.; Komatsu, T.; Terai, T.; Nagano, T. Development of a Fluorescein Analogue, TokyoMagenta, as a Novel Scaffold for Fluorescence Probes in Red Region. Chem. Commun. 2011, 47(14), 4162-4164. https://doi.org/10.1039/C1CC00078K.
(37) Koide, Y.; Urano, Y.; Hanaoka, K.; Terai, T.; Nagano, T. Evolution of Group 14 Rhodamines as Platforms for Near-Infrared Fluorescence Probes Utilizing Photoinduced Electron Transfer. ACS Chem. Biol. 2011, 6(6), 600-608. https://doi.org/10.1021/cb1002416.
(38) Grimm, J. B.; Sung, A. J.; Legant, W. R.; Hulamm, P.; Matlosz, S. M.; Betzig, E.; Lavis, L. D. Carbofluoresceins and Carborhodamines as Scaffolds for High-Contrast Fluorogenic Probes. ACS Chem. Biol. 2013, 8(6), 1303-1310. https://doi.org/10.1021/cb4000822.
(39) Fukazawa, A.; Suda, S.; Taki, M.; Yamaguchi, E.; Grzybowski, M.; Sato, Y.; Higashiyama, T.; Yamaguchi, S. Phospha-Fluorescein: A Red-Emissive Fluorescein Analogue with High Photobleaching Resistance. Chem. Commun. 2016, 52(6), 1120-1123. https://doi.org/10.1039/C5CC09345G.
(40) Lavis, L. D. Teaching Old Dyes New Tricks: Biological Probes Built from Fluoresceins and Rhodamines. Annual Review of Biochemistry 2017, 86(1), 825-843. https://doi.org/10.1146/annurev-biochem-061516-044839.
(41) Wang, L.; Frei, M. S.; Salim, A.; Johnsson, K. Small-Molecule Fluorescent Probes for Live-Cell Super-Resolution Microscopy. J. Am. Chem. Soc. 2019, 141(7), 2770-2781. https://doi.org/10.1021/jacs.8b11134.
(42) Karaman, O.; Alkan, G. A.; Kizilenis, C.; Akgul, C. C.; Gunbas, G. Xanthene Dyes for Cancer Imaging and Treatment: A Material Odyssey. Coordination Chemistry Reviews 2023, 475, 214841. https://doi.org/10.1016/j.ccr.2022.214841.
(43) Takakura, H.; Zhang, Y.; Erdmann, R. S.; Thompson, A. D.; Lin, Y.; McNellis, B.; Rivera-Molina, F.; Uno, S.; Kamiya, M.; Urano, Y.; Rothman, J. E.; Bewersdorf, J.; Schepartz, A.; Toomre, D. Long Time-Lapse Nanoscopy with Spontaneously Blinking Membrane Probes. Nat Biotechnol 2017, 35(8), 773-780. https://doi.org/10.1038/nbt.3876.
(44) Roth, A.; Li, H.; Anorma, C.; Chan, J. A Reaction-Based Fluorescent Probe for Imaging of Formaldehyde in Living Cells. J. Am. Chem. Soc. 2015, 137(34), 10890-10893. https://doi.org/10.1021/jacs.5b05339.
(45) Brewer, T. F.; Chang, C. J. An Aza-Cope Reactivity-Based Fluorescent Probe for Imaging Formaldehyde in Living Cells. J. Am. Chem. Soc. 2015, 137(34), 10886-10889. https://doi.org/10.1021/jacs.5b05340.
(46) Jia, S.; Ramos-Torres, K. M.; Kolemen, S.; Ackerman, C. M.; Chang, C. J. Tuning the Color Palette of Fluorescent Copper Sensors through Systematic Heteroatom Substitution at Rhodol Cores. ACS Chem. Biol. 2018, 13(7), 1844-1852. https://doi.org/10.1021/acschembio.7b00748.
(47) Ortiz, G.; Liu, P.; Naing, S. H. H.; Muller, V. R.; Miller, E. W. Synthesis of Sulfonated Carbofluoresceins for Voltage Imaging. J. Am. Chem. Soc. 2019, 141(16), 6631-6638. https://doi.org/10.1021/jacs.9b01261.
(48) Guo, Z.; Nam, S.; Park, S.; Yoon, J. A Highly Selective Ratiometric Near-Infrared Fluorescent Cyanine Sensor for Cyste-ine with Remarkable Shift and Its Application in Bioimaging. Chem. Sci. 2012, 3(9), 2760-2765. https://doi.org/10.1039/C2SC20540H.
(49) Pascal, S.; Haefele, A.; Monnereau, C.; Charaf-Eddin, A.; Jacquemin, D.; Le Guennic, B.; Andraud, C.; Maury, O. Expanding the Polymethine Paradigm: Evidence for the Contribution of a Bis-Dipolar Electronic Structure. J. Phys. Chem. A 2014, 118(23), 4038-4047. https://doi.org/10.1021/jp501358q.
(50) Liu, Y.; Zhou, J.; Wang, L.; Hu, X.; Liu, X.; Liu, M.; Cao, Z.; Shangguan, D.; Tan, W. A Cyanine Dye to Probe Mitophagy: Simultaneous Detection of Mitochondria and Autolysosomes in Live Cells. J. Am. Chem. Soc. 2016, 138(38), 12368-12374. https://doi.org/10.1021/jacs.6b04048.
(51) Lucjan Strekowski; J. Christian Mason; Martial Say; Hyeran Lee; Rajni Gupta; Maryam Hojat. Novel Synthetic Route To Ph-Sensitive 2,6-Bis(Substituted Ethylidene) Cyclohexanone/Hydroxycyanine Dyes That Absorb In The Visible/Near-Infrared Regions. Heterocyclic Communications 2005, 11(2), 129-134. https://doi.org/10.1515/HC.2005.11.2.129.
(52) Pascal, S.; Denis-Quanquin, S.; Appaix, F.; Duperray, A.; Grichine, A.; Guennic, B. L.; Jacquemin, D.; Cuny, J.; Chi, S.-H.; Perry, J. W.; Sanden, B. van der; Monnereau, C.; Andraud, C.; Maury, O. Keto-Polymethines: A Versatile Class of Dyes with Outstanding Spectroscopic Properties for in Cellulo and in Vivo Two-Photon Microscopy Imaging. Chem. Sci. 2016, 8(1), 381-394. https://doi.org/10.1039/C6SC02488B.
(53) $364 per 5 g of IR-780 from Thermo Scientifical Chemicals. https://www.thermofisher.com/order/catalog/product/H32150.0 6 (accessed 2024 Jan. 27).
(54) He, L.; Lin, W.; Xu, Q.; Ren, M.; Wei, H.; Wang, J.-Y. A Simple and Effective “Capping” Approach to Readily Tune the Fluorescence of near-Infrared Cyanines. Chem. Sci. 2015, 6(8), 4530-4536. https://doi.org/10.1039/C5SC00348B.
(55) Su, D.; Teoh, C. L.; Samanta, A.; Kang, N.-Y.; Park, S.-J.; Chang, Y.-T. The Development of a Highly Photostable and Chemically Stable Zwitterionic Near-Infrared Dye for Imaging Applications. Chem. Commun. 2015, 51(19), 3989-3992. https://doi.org/10.1039/C4CC08814J.
(56) Licha, K.; Welker, P.; Weinhart, M.; Wegner, N.; Kern, S.; Reichert, S.; Gemeinhardt, I.; Weissbach, C.; Ebert, B.; Haag, R.; Schirner, M. Fluorescence Imaging with Multifunctional Polyglycerol Sulfates: Novel Polymeric near-IR Probes Targeting Inflammation. Bioconjugate Chem. 2011, 22(12), 2453-2460. https://doi.org/10.1021/bc2002727.
(57) Li, D.-H.; Schreiber, C. L.; Smith, B. D. Sterically Shielded Heptamethine Cyanine Dyes for Bioconjugation and High Performance Near-Infrared Fluorescence Imaging. Angewandte Chemie International Edition 2020, 59(29), 12154-12161. https://doi.org/10.1002/anie.202004449.
(58) Levitz, A.; Marmarchi, F.; Henary, M. Synthesis and Optical Properties of Near-Infrared Meso-Phenyl-Substituted Symmetric Heptamethine Cyanine Dyes. Molecules 2018, 23(2), 226. https://doi.org/10.3390/molecules23020226.
(59) Rurack, K.; Spieles, M. Fluorescence Quantum Yields of a Series of Red and Near-Infrared Dyes Emitting at 600-1000 Nm. Anal. Chem. 2011, 83(4), 1232-1242. https://doi.org/10.1021/ac101329h.
(60) Cosco, E. D.; Lim, I.; Sletten, E. M. Photophysical Properties of Indocyanine Green in the Shortwave Infrared Region. ChemPhotoChem 2021, 5(8), 727-734. https://doi.org/10.1002/cptc.202100045.
(61) van der Wal, S.; Kuil, J.; Valentijn, A. R. P. M.; van Leeuwen, F. W. B. Synthesis and Systematic Evaluation of Symmetric Sulfonated Centrally CC Bonded Cyanine Near-Infrared Dyes for Protein Labelling. Dyes and Pigments 2016, 132, 7-19. https://doi.org/10.1016/j.dyepig.2016.03.054.

The present invention has been described with reference to certain preferred and alternative embodiments that are intended to be exemplary only and not limiting to the full scope of the present invention as set forth in the appended claim.

We claim:

1. A method of synthesizing a fluorophore, comprising the steps of:
converting a heptamethine dye to a keto-heptamethine dye; and
converting said keto-heptamethine dye to a 4′-aryl heptamethine dye.

2. The method of claim 1, wherein said heptamethine dye has the formula: 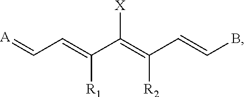
wherein:
X is selected from chloride, bromine, and iodine,
R₁ and R₂ are each independently selected from hydrogen, an alkyl, and a halogen, or R₁ and R₂ together complete a cycloalkenyl ring, a heterocyclyl ring, or a polycyclyl ring system, and
A and B are each independently selected from a bicyclic heterocycle, a tricyclic heterocycle, and a tetracyclic heterocycle.

3. The method of claim 2, wherein said heptamethine is IR-780, IR-783, IR-775, Chrom7, or IR-1061.

4. The method of claim 1, wherein said keto-heptamethine dye has the formula: 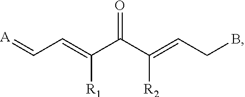
wherein:
X is selected from chloride, bromine, and iodine;
R₁ and R₂ are each independently selected from hydrogen, an alkyl, and a halogen, or R₁ and R₂ together complete a cycloalkenyl ring, a heterocyclyl ring, or a polycyclyl ring system, and
A and B are each independently selected from a bicyclic heterocycle, a tricyclic heterocycle, and a tetracyclic heterocycle.

5. The method of claim 1, wherein said keto-heptamethine dye is IR-780═O, IR-783═O, IR-775═O, Chrom7═O, or IR-1061═O.

6. The method of claim 1, wherein said 4′-aryl heptamethine dye has the formula: 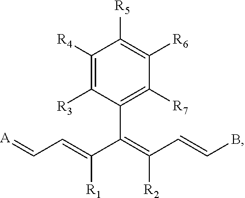
wherein:
R₁ and R₂ are each independently selected from hydrogen, an alkyl, and a halogen, or R₁ and R₂ together complete a cycloalkenyl ring, a heterocyclyl ring, or a polycyclyl ring system,
R₃-R₇ are independently selected from hydrogen, an alkyl, an alkoxy, a haloalkyl, an amine, and a halogen, and
A and B are each independently selected from a bicyclic heterocycle, a tricyclic heterocycle, and a tetracyclic heterocycle.

7. The method of claim 1, wherein said 4′-aryl heptamethine dye is IR780-Ph, IR780-Ph(Me), IR780-Ph(OMe), IR780-Ph(CH₂OH), IR780-Ph(COOH), IR780-Ph(CH₂StBu), IR780-Ph(2Me), IR780-Ph(2iPr), IR780-Ph(2CH₂OH), IR775-Ph(Me), IR813-Ph(Me), Chrom7-Ph(Me), IR1061-Ph(Me), or IR783-Ph(Me).

8. The method of claim 1, wherein said step of converting said keto-heptamethine dye to said 4′-aryl heptamethine dye comprises the step of reacting said keto-heptamethine dye with an aryl anion.

9. The method of claim 8, wherein said aryl anion is generated via reduction of an aryl halide with a metal or via a metal-halo exchange reaction.

10. The method of claim 8, wherein said aryl anion is an aryl lithium reagent, an aryl Grignard reagent, or combinations thereof.

11. The method of claim 10, wherein said aryl lithium reagent is prepared by reacting n-BuLi with an aryl halide selected from the group consisting of bromobenzene, 2-bromotoluene, 1-bromo-2-isopropylbenzene, 2-bromoanisole, ((2-bromo-3-methylbenzyl)oxy)trimethylsilane, tert-butyl 2-bromobenzoate, or (2-bromobenzyl)(tert-butyl)sulfane.

12. The method of claim 10, wherein said aryl lithium reagent is prepared by reacting t-BuLi with 2-bromo-1,3-dimethylbenzene, 1,3-Bis((allyloxy)methyl-2-bromobenzene, or 2-bromo-1,3-diisopropylbenzene.

13. The method of claim 1, wherein said step of converting said heptamethine dye to said keto-heptamethine dye comprises the step of reacting said 4′-chloro heptamethine dye with sodium acetate and dimethylformamide.

14. The method of claim 1, wherein said step of converting said heptamethine dye to said keto-haptamethine dye comprises the step of reacting said 4′-chloro heptamethine dye with NHS and DIPEA.
